(19) United States
(12) Patent Application Publication
(10) Pub. No.: US 2011/0246081 A1
(43) Pub. Date: Oct. 6, 2011

(54) Metabolomics-Based Identification of Disease-Causing Agents

(75) Inventors: Jeffrey Skolnick, Roswell, GA (US); Adrian K. Arakaki, Marietta, GA (US); Susana Noemi Do Brito Afonso, Santa Fe (AR); John McDonald, Arnoldsville, GA (US); Roman Mezencev, Atlanta, GA (US); Nathan Bowen, Atlanta, GA (US)

(73) Assignee: GEORGIA TECH RESEARCH CORPORATION, Atlanta, GA (US)

(21) Appl. No.: 12/681,959

(22) PCT Filed: Oct. 15, 2008
(86) PCT No.: PCT/US08/80002

§ 371 Date: May 13, 2011

(30) Foreign Application Priority Data
Oct. 15, 2007 (US) 60979932
Oct. 18, 2007 (US) 60980954
Nov. 20, 2007 (US) 60989233

(51) Int. Cl. G06F 19/00; A61K 38/14

(52) U.S. Cl. 702/20; 514/20.9

(57) ABSTRACT

A method, computer-readable medium, and system for identifying one or more metabolites associated with a disease, comprising: comparing gene expression data from diseased cells to gene expression data from control cells in order to deduce genes that are differentially-regulated in the diseased cells relative to the control cells; based on enzyme function and pathway data for all human metabolites that utilize the genes that are differentially-regulated in the disease cells, identifying one or more metabolites whose intracellular levels are higher or lower in diseased cells than in control cells, and thereby associating the one or more metabolites with the disease.

### CLAIM OF PRIORITY

This application claims the benefit of priority under 35 U.S.C. §119(e) to U.S. provisional application Ser. Nos. 60/979,932, filed Oct. 15, 2007, and 60/980,954, filed Oct. 18, 2007, and 60/989,233, filed Nov. 20, 2007, all of which are incorporated herein by reference in their entirety.

### TECHNICAL FIELD

The technology described herein relates to methods for determining metabolites that can be used as agents and/or targets for the therapeutic treatment of disease. The levels of one or more metabolites identified using these methods can be manipulated to increase or decrease the endogenous and/or intracellular levels of these metabolites by, for example, administration of the metabolites themselves, inhibition/activation of relevant enzymes, and/or inhibitors/activators of specific transporters.

### BACKGROUND

Today the search for disease cures centers on identifying key molecular determinants of the disease. If such molecules—and the roles they play—can be identified, then regulation of their concentration, or inhibition of their function, may be successful routes to a disease therapy. In the complex biochemical interplay that underlies most disease conditions, many molecules play more than one role—sometimes a useful role as well as a detrimental role—and many molecules are created and altered as the biochemical machinery performs its task. Molecules that are created during metabolic processes—metabolites—may prove useful targets in developing many disease therapies.

Elucidating the metabolic changes exhibited by cancer cells is important not only for diagnostic purposes, but also to more deeply understand the molecular basis of carcinogenesis, which could lead to novel therapeutic approaches. Certain metabolic processes may play fundamental roles in cancer progression by regulating the expression of oncogenes or modulating various signal transduction systems. The significance of other metabolic phenotypes observed in cancer is more controversial, such as the shift in energy production from oxidative phosphorylation (respiration) to aerobic glycolysis, which is known as the Warburg effect. The prevailing view recently has been that the Warburg effect is a consequence of the cancer process (secondary events due to hypoxic tumor conditions) rather than a mechanistic determinant, as originally hypothesized. Recently, however, a different picture of the role of metabolic changes in tumorigenesis has emerged. For example, the dichloroacetate-induced reversion from a cytoplasm-based glycolysis to a mitochondria-located glucose oxidation inhibits cancer growth. This suggests that a glycolytic shift is a fundamental requirement for cancer progression.

Changes in intracellular concentrations of certain metabolites can influence the rate of cancer cell growth. A metabolite can exert this effect by acting as a signaling molecule, a role that does not preclude other important cellular functions. For instance, diacylglycerol, a lipid that confers specific structural and dynamic properties to biological membranes and serves as a building block for more complex lipids, is also an essential second messenger in mammalian cells whose dysregulation contributes to cancer progression. Similarly, structural components of cell membranes, such as the sphingolipids ceramide and sphingosine, are also second messengers with antagonizing roles in cell proliferation and apoptosis. Pyridine nucleotides constitute yet another example, having well characterized functions as electron carriers in metabolic redox reactions and roles in signaling pathways. In particular, NAD+ modulates the activity of sirtuins, a recently discovered family of deacetylases that may contribute to breast cancer tumorigenesis. Arginine is yet another metabolite involved in numerous biosynthetic pathways that also has a fundamental role in tumor development, apoptosis, and angiogenesis.

Cellular metabolites can also be involved in the control of cell proliferation by directly regulating gene expression. Signaling pathway-independent modulation of gene expression by metabolites can occur in several ways. For example, metabolites can bind to regulatory regions of certain mRNAs (riboswitches), inducing allosteric changes that regulate the transcription or translation of the RNA transcript, however, this type of direct metabolite-RNA interaction has not yet been detected in humans. In another example, transcription factors can be activated upon metabolite binding (e.g., binding of steroid hormones to the estrogen receptor transcription factor induces gene expression events leading to breast cancer progression). In yet another example, metabolites can be involved in epigenetic processes such as post-translational modification of histones that regulate gene expression by changing chromatin structure. The modulation of the rate of histone acetylation by nuclear levels of acetyl-CoA is an example of metabolic control over chromatin structure that involves epigenetic changes linked to cell proliferation and carcinogenesis.

Manipulation of specific metabolic pathways has been the basis of several anticancer therapies that have been proposed based on experimental evidence, that are subject to validation in clinical trials, and/or that are currently in use. An exemplary anticancer therapy that was proposed based on experimental evidence is the inactivation of the metabolic enzyme KIAA1363 which decreased the rate of tumor growth in vivo. Several anticancer treatments that exploit the antiproliferative action of ceramide are examples of therapies based on the pharmacological manipulation of a metabolic pathway that are currently in clinical trials. A metabolite-based therapy, that has been used since 1970 for acute lymphoblastic leukemia, and has also applied to ovarian cancer and other tumors, consists of depleting circulating asparagine by administration of the bacterial enzyme L-asparaginase.

To date, however, the search for metabolites that have a direct connection to a particular disease state has been haphazard. Rather than making reasonable predictions of the metabolites that are likely to be involved in a particular disease, researchers still rely on fortuitous discoveries.

### SUMMARY

In general, preventive and therapeutic anticancer approaches based on the pharmacological manipulation of metabolism aim to increase or decrease the intracellular levels of certain metabolites by, for example, administration of either the metabolites themselves, inhibitors/activators of relevant enzymes, and/or inhibitors/activators of specific transporters.

A method for identifying one or more metabolites associated with a disease, the method comprising: obtaining a set of gene-expression data from diseased cells of an individual with the disease; obtaining a reference set of gene-expression data from control cells; assigning an expression status to each gene in the gene expression data that encodes a gene product, wherein the expression status for each gene is one of: up-regulated in the diseased cells relative to the control cells; down-regulated in the diseased cells relative to the control cells; expressed by both the diseased cells and the control cells at statistically indistinguishable levels; and not expressed by both the diseased cells and the control cells; determining the effects of gene products on metabolite levels for each metabolite in a list of human metabolites: identify a set of gene products that have an effect on the metabolite; using the expression status for the gene that encodes each gene product that has an effect on the metabolite, predict whether an intracellular level of the metabolite in the diseased cells is increased or decreased relative to its level in control cells; identifying one or more of: those metabolites whose intracellular level is predicted to be lower in diseased cells than in control cells; and those metabolites whose intracellular level is predicted to be higher in diseased cells than in control cells, as associated with the disease.

A method for identifying one or more metabolites associated with a disease, the method comprising: comparing gene expression data from diseased cells to gene expression data from control cells in order to deduce genes that are differentially-regulated in the diseased cells relative to the control cells; based on enzyme function and pathway data for all human metabolites that utilize the genes that are differentially-regulated in the disease cells, identifying one or more metabolites whose intracellular levels are lower in diseased cells than in control cells, and thereby associating the one or more metabolites with the disease.

A method for identifying one or more metabolites associated with a disease, the method comprising: comparing gene expression data from diseased cells to gene expression data from control cells in order to deduce genes that are differentially-regulated in the diseased cells relative to the control cells; based on enzyme function and pathway data for all human metabolites that utilize the genes that are differentially-regulated in the disease cells, identifying one or more metabolites whose intracellular levels are higher in diseased cells than in control cells, and thereby associating the one or more metabolites with the disease.

A method of determining a metabolite-based disease therapy, the method comprising: identifying one or more metabolites associated with the disease, by the methods described herein, and administering said one or more metabolites to an individual with the disease.

A method of treating an individual with a disease, the method comprising: administering to the individual a metabolite identified as associated with the disease by the methods described herein, in an amount sufficient to produce a therapeutic effect.

A method of determining a metabolite-based disease therapy, the method comprising: identifying one or more metabolites associated with the disease, by the methods described herein; and administering one or more drugs to change the levels of said one or more metabolites to an individual with the disease.

The present technology further comprises computer systems configured to carry out the methods described herein in whole or in part, and to provide results of said methods to a user, as for example on a display or in the form of a printout.

The present technology further comprises computer-readable media, encoded with computer-executable instructions for carrying out the methods described herein in whole or in part, when operated on by a suitably configured computer.

When it is stated that a computer system is configured to carry out a method in whole or in part, or that a computer readable medium is configured with instructions for carrying out a method in whole or in part, it is understood to mean that one or more steps of the method is carried out, other than by the computer or computer system. For example, obtaining gene expression data may be obtained manually and read into the computer, or written on to a computer-readable medium.

The details of one or more embodiments of the invention are set forth in the accompanying drawings and the description herein. Other features, objects, and advantages of the invention will be apparent from the description and drawings, and from the claims.

### BRIEF DESCRIPTION OF THE DRAWINGS

FIG. 1 is a flow chart depicting a method for a metabolomics-based method of identifying one or more metabolites associated with a disease that may have potential as therapeutic agents and/or targets, in accordance with some embodiments.

FIG. 2 is a flow chart depicting a method for assigning an expression status to genes, based on gene-expression data, in accordance with some embodiments.

FIG. 3A depicts a portion of an exemplary genetic-metabolic matrix, in accordance with some embodiments.

FIG. 3B depicts a portion of an exemplary genetic-metabolic matrix that includes information about the differential expression of gene products, in accordance with some embodiments.

FIGS. 4A and 4B depict exemplary metabolites, gene products that they interact with, and differential expression information about the gene products, in accordance with some embodiments.

FIG. 5 is a flow chart depicting a method for determining the level of metabolites (e.g., increased, decreased, or unknown) in diseased cells relative to control cells, in accordance with some embodiments.

FIG. 6 depicts an exemplary computer system that can perform the methods described herein, in accordance with some embodiments.

FIGS. 7A-7D depict charts showing metabolites whose concentrations were increased in Jurkat cells to test the effect on growth, in certain embodiments.

FIGS. 8A-8C depict charts showing metabolites whose concentrations were increased in OVCAR-3 cells to test the effect on growth, in other embodiments.

Like reference symbols in the various drawings indicate like elements.

### DETAILED DESCRIPTION

In some embodiments, a metabolomics-based system, such as a computer-based system, that utilizes various data such as metabolic data, can be used to identify one or more metabolites associated with a disease that may have potential as agents and/or targets for therapeutic treatment. The system described here can use a combination of gene-expression data and the relationships between metabolites and gene products to make predictions on the levels of metabolites in diseased cells compared to control cells.

By ‘gene product’ as used herein, is meant molecules, in particular biochemical molecules such as oligonucleotides (DNA, RNA, etc.) or proteins, resulting from the expression of a gene. A measurement of the amount of gene product can be used to infer how active a gene is. Abnormal amounts of gene product can be correlated with diseases, such as the overactivity of oncogenes which can cause cancer, the overexpression of Interleukin-10 which can induce symptoms in virus-induced asthma, and the underexpression of certain genes in early Parkinson's disease. Exemplary gene products of particular interest herein include small molecule transporters, and enzymes, because of their respective involvement in metabolic pathways.

Computational analysis of gene-expression data acquired from both diseased and control cells can determine gene products that are over or under expressed in diseased cells. Data indicative of the relationships between metabolites and gene products, such as data determined from biochemical pathways, enzyme function prediction, and the like, can be used to relate the effect of differential expression on metabolite levels. Considering the relationships and the gene-expression data, predictions can be made on the effect of a disease state on the endogenous and/or intracellular level of metabolites. As used herein, it is to be understood that “intracellular” includes any material that can penetrate a cell membrane, and therefore includes synthetic (non-naturally occurring) species such as pharmaceuticals. “Endogenous” includes those materials expressed, synthesized, or otherwise made naturally within cells.

The metabolites that are predicted to exist at different levels in diseased cells (relative to control cells, such as from a healthy individual) can be further evaluated as potential agents and/or targets, for therapeutic treatments. For example, metabolites that exist at decreased levels in cancer cells, relative to control cells, can be potential agents for anticancer therapies. In which case, one or more metabolites can be supplemented to raise the cellular levels of each of these metabolites to within normal physiological ranges, for the purpose of restoring normal cell function. Similarly, metabolites that exist at increased levels in cancer cells can be targets for anticancer therapies. In this example, activation or inhibition of key enzymes could be used to lower cellular levels of each of these metabolites to within normal physiological levels. In either case, the systems and methods described herein can be used to identify which metabolites, from the larger group of known physiological metabolites, are likely to be agents and/or targets for therapeutic treatments.

Cellular metabolites can be produced and/or consumed by enzymes, bind to regulatory regions of mRNA, activate transcription factors, and/or regulate gene expression through post-translational modification. In diseased cells, certain genes can be over/under expressed leading to increased/decreased levels of one or more metabolites. In some circumstances, it may be possible to restore normal cell function in a diseased cell by returning one or more metabolite levels back to a normal range. In circumstances where a metabolite exists at a lower level in diseased cells, relative to control cells, raising the level of metabolite may have therapeutic value. Conversely, lowering the metabolite level in diseased cells exhibiting increased metabolite levels may also have therapeutic value. One method for determining possible therapeutic agents and/or targets would be to compare the actual intracellular levels of every human metabolite as they exist in normal and diseased states. Metabolites that exist in differential levels between the diseased and control cells could be candidates for further testing to determine their therapeutic value. Currently, however, there is no feasible way to implement such large-scale biochemical assays. As an alternative, gene expression studies, known to individuals skilled in the art, coupled with information relating to biochemical pathways (e.g., gene product function, enzyme function, and the like), can be utilized to predict metabolites that may exist at increased/decreased levels in diseased cells, relative to control cells. These predicted metabolites can be further evaluated, using methods known to individuals skilled in the art, to determine their value as agents and/or targets of therapeutic treatments.

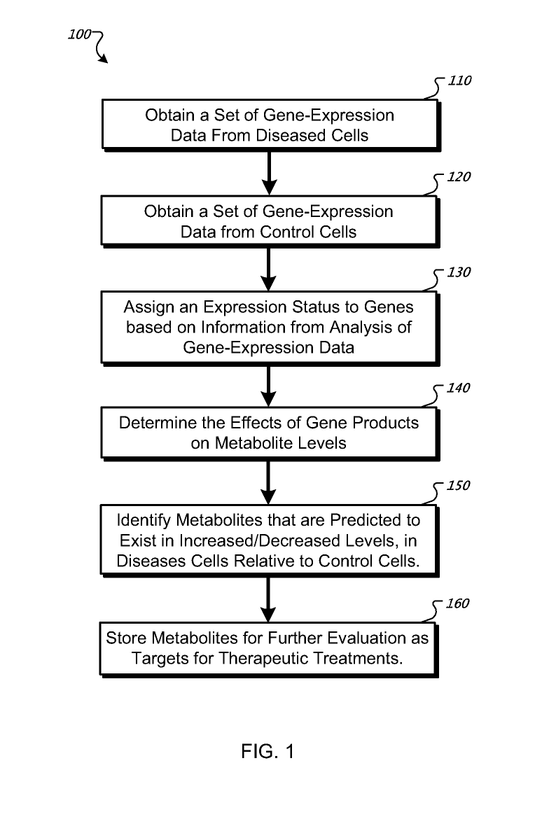

Referring now to FIG. 1, a process 100 for identifying metabolites associated with a disease, which may have potential as agents and/or targets for therapeutic treatment, can be included in a computational method, such as encoded on a computer-readable medium, in whole or in part, and performed on a computer, in whole or in part. In some embodiments, the process 100 can execute operation 110, causing the metabolomics-based system to obtain gene-expression data from diseased cells. For example, gene expression data can be obtained from gene expression studies that can be performed on Jurkat cells (an immortalized line of T lymphocyte cells derived from an acute lymphoblastic leukemia patient). In other embodiments, gene expression studies can be performed on cells obtained from one or more individuals with a disease. In general, such gene expression studies can be performed in a way that is known to one skilled in the art using, for example, DNA microarray technology and corresponding software, the results of which can be stored for later retrieval by the process 100 during operation 110.

In operation 120, the metabolomics-based system can obtain gene-expression data from studies performed on control cells. For example, gene-expression data can be obtained from previously performed gene expression studies of non-diseased cells that are similar in type to the cells from which the data in operation 110 was acquired. In other embodiments, studies can be performed on non-diseased cells, of a similar type, to obtain the gene-expression data. In operation 130, a differential analysis of the gene-expression data, obtained during operations 110 and 120, can be performed for the purpose of assigning an expression status to each of the genes. For example, genes can be assigned a status such as up-regulated in the diseased cells, down-regulated in the diseased cells, similarly expressed in both the diseased and control cells, or not expressed in both the diseased and control cells.

In operation 140, the effects of gene products on metabolite levels are determined from, for example, existing databases, computational enzyme-function prediction, or the like. In some embodiments, gene products and associated metabolites can be assigned to steps in metabolic pathways. Information from databases can be retrieved and analyzed to identify metabolite/gene product interactions found in the database. In other techniques, the function of, and metabolites related to, proteins with currently unknown function can be inferred using, for example, similarity to proteins with known functions. These relationships can then be used to determine the effect that a particular gene product has on a metabolite. For example, if the gene product (e.g., an enzyme) is determined to catalyze the production of a certain metabolite, it can be deduced that the gene product causes an increase in the intracellular level of the metabolite. Conversely, if the gene product is determined to transport the metabolite out of the intracellular space (e.g., into storage vesicles), it can be deduced that the gene product causes a decrease in the intracellular level of the metabolite. In some embodiments, this information can be determined during operation 140. In other embodiments, some or all of this information can be determined at a previous time and retrieved during operation 140.

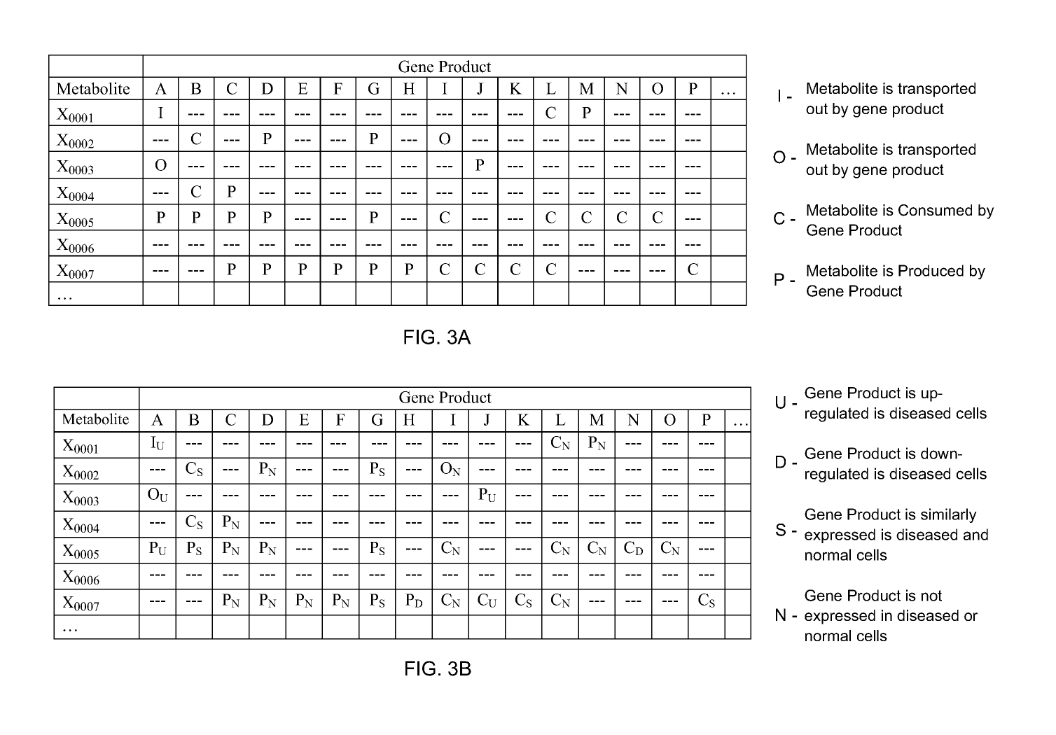

In operation 150, the results of the previously described operations can be used to identify metabolites that are predicted to exist in increased/decreased levels in diseased cells relative to control cells. For example, the metabolomics-based system can create a genetic-metabolic matrix including all metabolites and their known relationships to gene products. An example of such a matrix can be found in FIG. 3A. The matrix can then be annotated to include the results of a differential analysis of gene-expression data, such as the expression statuses assigned during operation 130 (described in connection with FIG. 1).

For example, metabolite X may be known to be produced by enzyme A (which is decreased in diseased cells) and consumed by enzyme F (which is increased in diseased cells), where the relationships between metabolite X and enzymes A and F were determined during operation 140 and the differential levels of enzyme A and F in diseased cells, compared to control cells, were determined during an analysis of gene-expression data, such as during operation 130. From the relationships between metabolites and gene products and the expression status of the genes that code for these gene products, the metabolomics based system can predict the levels of metabolites in diseased cells relative to control cells. For example, the metabolite X described previously, because it is produced at lower levels in the diseased cells (due to the decreased expression of the gene that produces enzyme A) and consumed at higher levels in the diseased cells (due to the increased expression of the gene that produces enzyme B), can be predicted to exist at lower levels in the diseased cells. Information indicative of the level of metabolites in diseased cells compared to control cells is stored during operation 160 for display and/or future evaluation as potential agents and/or targets for therapeutic treatments.

In some embodiments, the metabolomics-based system can be used to identify agents and/or targets for anti-cancer therapies. For example, studies of ovarian cancer cells and normal ovarian cells can be used to predict metabolites that exist in different levels in the cancer cells (relative to normal cells). One or more of the metabolites, predicted to exist in differential levels, can then be evaluated as agents and/or targets for potential anti-cancer therapies. Metabolites that exist at decreased levels in cancer cells can be supplemented to raise intracellular levels to a near normal range, while metabolites that exist at increased levels can be targets for therapies that decrease the intracellular levels of the metabolites. Some therapies may involve only a single metabolite, while other therapies may involve multiple metabolites concurrently. In cases where multiple metabolites are involved concurrently, some metabolites may be supplemented, while other metabolites levels may be decreased. In one example, a metabolomics-based system such as described herein was used to predict that Seleno-L-methionine exists at decreased levels in ovarian cancer cells (e.g., Hey-A8 and Hey-A8 MDR cells). Subsequently, supplementation of Seleno-L-methionine was shown in vitro to inhibit the growth of Hey-A8 and Hey-A8 MDR cells.

In some embodiments, the metabolomics-based system can be used to identify metabolites that may have potential as agents and/or targets for therapeutic treatment. In one embodiment described herein, analysis of expression data, acquired through gene expression studies of diseased and control cells, can be used to identify genes that are expressed at different levels in diseased cells and control cells. This information can be combined with, for example, knowledge of biochemical pathways (e.g., the relationships between metabolites and gene products) and/or the predicted function of gene products (whose function is not known) to predict the relative level of metabolites in diseased cells compared to the level found in control cells.

For example, the knowledge that enzyme A (which produces metabolite X) is expressed at a lower level in a diseased cell and that enzyme B (which consumes metabolite X) is expressed at a higher rate in the diseased cell could lead one to predict that the level of metabolite X found in the diseased cell would be lower than the level in a normal, non-diseased cell. This prediction could indicate that metabolite X is a potential agent for therapeutic treatment. In this case, where a metabolite is predicted to exist at lower levels in a diseased cell, the metabolite itself could be supplemented to raise the physiological levels of the metabolite up to a normal range. Conversely, where a metabolite is predicted to exist at higher levels in a diseased cell, the metabolite could be a target for other therapies that lower the levels of the metabolite (e.g., activation or inhibition of key enzymes). In either case, the system described here can be used to identify metabolites, from the larger group of known physiological metabolites, which could be further evaluated, by other techniques, as agents and/or targets for therapeutic treatments.

To determine gene products that are expressed at different levels in diseased and control cells, gene expression studies (using methods known to individuals skilled in the art) can be performed on diseased and control cells. Based on the results of the expression studies, each gene can be classified into one of four possible groups: Gᵤₚ, indicating that the gene is up-regulated in diseased cells relative to control cells; G_{down}, indicating that the gene is down-regulated in diseased cells relative to control cells; Gₛᵢₘᵢₗₐᵣ, indicating that the levels in both diseased and control cells were statistically indistinguishable; and Gₙₒₙₑ, indicating that the gene was not expressed in either of the control or diseased cells. Exemplary information that can be used to classify genes includes data (e.g., signal intensities, presence calls, and the like) obtained through DNA microarray technology, serial analysis of gene expression (SAGE) technology, PCR based technologies, and the like.

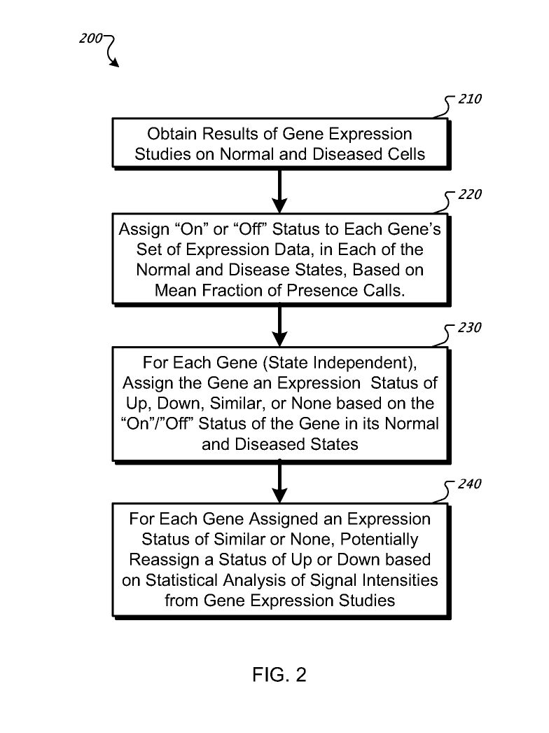

Referring now to FIG. 2, a process 200 can be performed by a metabolomics-based system, such as including a suitably configured computer, to assign an expression status to individual genes based on, for example, gene-expression data. In some embodiments, the process 200 is exemplary of operations that can be performed by the metabolomics-based system during operations 110-130 (described in connection with FIG. 1). Referring to the process 200, in operation 210, the metabolomics-based system can obtain gene-expression data (e.g., in micro-array format) performed on diseased and control cells. The gene expression studies performed, to obtain the data, utilize technologies that can quantify the level of gene expression in a cell (e.g., DNA microarray, serial analysis of gene expression, and the like). In some embodiments, the gene-expression data for both the diseased and control states can be determined from tissue samples obtained from a single individual. In other embodiments, one or more of the sets of gene-expression data can come from cell lines cultured in vitro. In still other embodiments, some of the data can come from previously performed gene expression studies.

In some embodiments, the gene-expression data obtained from studies of the diseased and control cells can be utilized, in operation 220, to assign an “on” or “off” status to each gene's set of expression data. This status can be assigned to every gene in each of the diseased and normal cells. In this way, each gene will have a status for the diseased and the non-diseased states. For example, the mean fraction of presence calls generated by the Affymetrix MICROARRAY SUITE 5.0 software can be used to assign a status of “on” or “off” to each gene in each expression study. In some embodiments, for genes where the mean fraction of presence calls labeled as “marginal” or “absent” in the corresponding probe sets is at least 80%, an “off” status is provisionally assigned to the gene, otherwise, an “on” status is assigned to the gene. This process is repeated until all genes have a provisional assignment, of “on” or “off”, for both of the studied conditions (e.g., control cells and diseased cells).

For example, gene A, whose expression levels were measured in both the study of the control cells and diseased cells, can be assigned a status for each state, where the status of the gene A in the non-diseased state is independent of the status of gene A in the diseased state, and vice versa. In other words, gene A in the diseased state can be assigned a status of “on” based on the results of the expression study of the diseased cells, while gene A in the non-diseased state can be assigned a status of “off” based on the results of the expression study of the control cells.

In operation 230, for all genes that have been assigned either an “on” or “off” status for both the control and the diseased states, each gene can be initially assigned an expression status of Gᵤₚ, G_{down}, Gₛᵢₘᵢₗₐᵣ, or Gₙₒₙₑ, based on the previously assigned statuses of the diseased and non-diseased states. A gene is assigned a Gᵤₚ expression status, indicating that the gene is up-regulated in diseased cells relative to control cells, if the status of the gene in the control cells is “off” and the status of the gene in the diseased cells is “on”. A gene is assigned a G_{down} expression status, indicating that the gene is down-regulated in diseased cells relative to control cells, if the status of the gene in the control cells is “on” and the status of the gene in the diseased cells is “off”. A gene is assigned a expression status, indicating that the levels of the gene in both diseased and control cells were statistically indistinguishable, if the status of the gene in control cells is “on” and the status of the gene in the diseased cells is “on”. A gene is assigned a Gₙₒₙₑ expression status, if the status of the gene in the control cells and the diseased cells is “off”.

In operation 240, additional tests can be applied to each of the genes with either a Gₛᵢₘᵢₗₐᵣ, or Gₙₒₙₑ expression status, for the purpose of potentially re-assigning their status. For example, differential expression (e.g., differences between the expression levels of the genes in control cells and the diseased cells, as measured during the expression studies) can be used to re-assign the expression status of genes that were previously assigned Gₛᵢₘᵢₗₐᵣ or Gₙₒₙₑ expression statuses. For genes classified as either Gₛᵢₘᵢₗₐᵣ or Gₙₒₙₑ, if the signal intensities in the diseased and control samples exhibit a statistically significant difference (e.g., in at least 40% of the corresponding probe sets, as evaluated by an ANOVA two-tailed test with P<0.005), the genes can be re-assigned the expression status of Gᵤₚ or G_{down}, depending on whether the gene is up-regulated in the diseased sample or down-regulated in the diseased sample, respectively. The expression statuses of the genes can be used later by the metabolomics-based system to predict the levels of metabolites in diseased cells compared to the levels in control cells. In alternate embodiments, each gene can be initially assigned an expression status (as in operation 230) and further re-assigned a new status (as in operation 240) before assigning a status to additional genes. While some exemplary criteria used to assign an expression status was described here, it remains within the scope of the method to utilize other criteria, in addition or in the alternative to those described here, to assign one or more expression statuses to genes. For example, different statistical tests, at different confidence levels, can be utilized to assign one of more or less than four expression statuses. In another example, genes may be annotated with quantitative information indicative of differential expression. A gene could be annotated with information that includes the percentage change between the non-diseased and diseased states of the cell (e.g., the gene is expressed at a 47% higher rate in the diseased cells than in the control cells, the gene is expressed at a 37% lower rate in the diseased cells than in the control cells, or the like). In yet another example, genes that are assigned an expression status can also be assigned confidence information (e.g., the gene is expressed at a higher rate in the diseased cells than in the control cells at a 58% confidence level, or the like).

In some embodiments, information determined about genes (e.g., which status of Gᵤₚ, G_{down}, Gₛᵢₘᵢₗₐᵣ, and Gₙₒₙₑ the genes are assigned) is used to estimate the potential effects of the differential expression, if any, on the endogenous and/or intracellular levels of metabolites. To do so, connections can be determined between gene products and metabolites. One such source of data connecting gene products and metabolites is information about metabolic pathways. Information regarding human metabolic pathways is available, for example, from existing databases, in the form of pathway maps. The pathway maps can be available as graphical images and also as markup language files that facilitate the parsing of relevant biological data. The biochemical reactions, including for example, information about substrates, products, direction/reversibility, and associated enzyme-coding genes can be extracted from the metabolic pathway maps and organized in such a way as to assist in predicting how the effects of differential gene expression affects endogenous and/or intracellular metabolite levels.

In some embodiments, such as the one described herein, the markup language files can be retrieved from a database, and necessary information extracted from these files when it is needed to estimate the potential effects of the differential expression on the endogenous and/or intracellular levels of metabolites. In other embodiments, this retrieval and extraction of data can be done at an earlier time and the results of this retrieval and extraction can be used for more than one set of predictions. Put another way, the files can be downloaded and the data can be extracted one or more times (e.g., weekly, monthly, on an on-demand basis, or the like), stored, and retrieved for later use by the metabolomics-based system to identify potential therapeutic agents and/or targets. However obtained, this data can be combined with gene-expression data from diseased and control cells to construct a genetic-metabolic matrix (e.g., during operation 140), an example of which is depicted in FIG. 3A. This matrix indicates, for each metabolite, which specific gene products affect that metabolite. This genetic-metabolic matrix can be further annotated (e.g., during operation 150) to include the differential expression status assigned in the previous section (an example of which is depicted in FIG. 3B). For example, for each metabolite considered, the gene products that affect that particular metabolite are stored, along with differential expression data (e.g., which expression group the gene belongs to), if available.

In some examples, particular metabolites are excluded from the genetic-metabolic matrix. Reasons to exclude a metabolite from the matrix can include, for example, that the metabolite is non-physiological, that the metabolite is ubiquitous, or that the metabolite participates in reactions that are mainly catalyzed by orphan human enzymes (well defined enzyme activities for which no sequence is known). Exemplary non-physiological metabolites (e.g., ecgonine and parathion) can include metabolites that only participate in reactions pertaining to the biosynthesis of secondary metabolites, the biodegradation and metabolism of xenobiotics, and the like. Ubiquitous metabolites (e.g., H₂O, ATP, NAD(+)(P), O₂, or the like) often carry out generic roles in many reactions and can be defined as those that are involved as substrate or product in twenty (20) or more reactions. Referring to the third exclusion category previously mentioned (the metabolite participates in reactions that are mainly catalyzed by an orphan human enzyme), the number of reactions where a metabolite m acts as a substrate or product in human metabolic pathways can be defined as Nr_{m,human} and the number of reactions where the metabolite m acts as a substrate or product in reference (e.g., non organism specific) metabolic pathways can be defined as Nr_{m,ref}. If Nr_{m,human}/Nr_{m,ref}<0.5, then the metabolite m can belong to the third exclusion category (e.g., the metabolite participates in reactions that are mainly catalyzed by orphan human enzymes). The metabolites determined to be part of the third exclusionary category may be excluded because the reactions are due to orphan enzymes, the reactions only occur in other organisms, or the reactions occur in humans but have not yet been detected. For example, the metabolite 1-alkyl-sn-glycero-3-phosphate is excluded because out of four enzymes that use it as substrate or product, two, EC 2.3.1.105 and EC 1.1.1.101, are orphans in human, and one, EC 2.7.1.93, has only been found in rabbits. The metabolomics-based system can use the methods described herein (e.g., during operation 150) to generate a matrix such as the one depicted in FIG. 3B.

In some embodiments, the metabolomics-based system can utilize information indicative of relationships between metabolites and gene products together with gene-expression data to predict the relative levels of metabolites in diseased cells, relative to control cells. For example, based on information contained in a genetic-metabolic matrix annotated with differential gene-expression data, the system can predict which metabolites are expected to exist at higher levels in diseased cells, which metabolites are expected to exist at lower levels in diseased cells, and which metabolites are unknown as to their levels in diseased cells compared to control cells. Based on the rules applied, these predictions can also include a confidence level indicating the degree of confidence associated with the prediction. In this way, metabolites that are predicted to exist at different levels in diseased cells, relative to cells, can be prioritized based on the level of confidence associated with the prediction, such that future testing of the metabolites as therapeutic agents and/or targets can be prioritized based on the confidence level of the predictions.

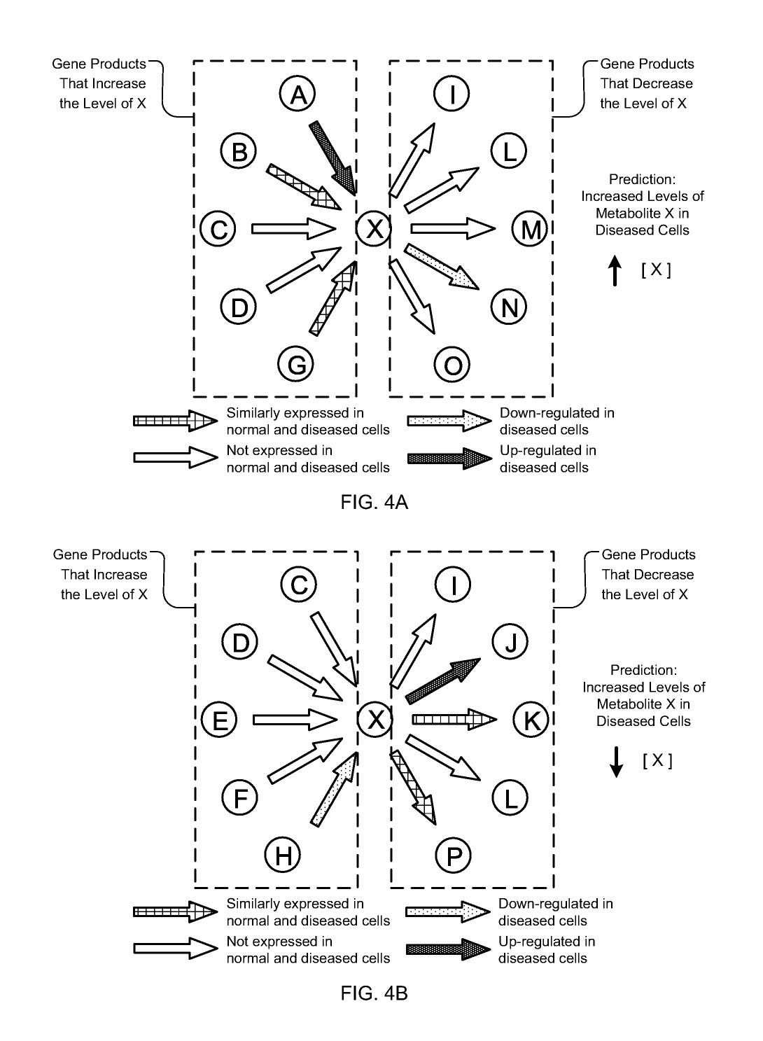

Referring to FIGS. 4A and 4B, the effects of gene products on metabolite levels, along with differential gene-expression data, can be depicted graphically. For example, as depicted in FIGS. 4A and 4B, some gene products may increase the endogenous levels of a metabolite by producing the metabolite and/or increasing the intracellular level of the metabolite by transporting metabolite into the cell. Conversely, other gene products may decrease the intracellular levels of a metabolite by transporting the metabolite out of the cell and/or decreasing the intracellular level of the metabolite by consuming metabolite in enzymatic reactions. Assessment of the cumulative effect of these relationships along with information indicative of the expression levels of gene products can be used to predict the level of metabolites in diseased cells compared to control cells. Generally speaking, higher levels of gene products that increase the level of a metabolite and lower levels of gene products that decrease the level of a metabolite each have the effect of increasing the endogenous/intracellular level of that metabolite. Conversely, lower levels of gene products that increase the level of a metabolite and higher levels of gene products that decrease the level of a metabolite each have the effect of decreasing the endogenous/intracellular level of that metabolite. In diseased cells, genes that are over or under expressed can be identified and used to predict metabolites that may exist at higher or lower levels in these cells.

Referring to the embodiment depicted by FIG. 4A, the genes that code for gene products C, D, I, L, M, O are not expressed in either the control or diseased cells, and thus have no effect on the endogenous/intracellular levels of metabolite X. The genes that code for gene products B and G are expressed in similar levels in diseased and control cells, and thus are also predicted to have little or no effect on the levels of metabolite X. However, the gene that codes for product A, which increases the level of metabolite X, is expressed at higher levels in diseased cells and the gene that codes for product N, which decreases the level of metabolite X, is expressed at lower levels. The predicted effect of each of these differences in expression is to increase the endogenous/intracellular levels of metabolite X in the diseased cells. In this example, the cumulative effect of the differential levels of gene products is predicted to have the effect of increasing the endogenous/intracellular levels of metabolite X in diseased cells compared to control cells.

In another embodiment, depicted by FIG. 4B, the cumulative effect of the differential levels of gene products is predicted to have the effect of decreasing the endogenous/intracellular levels of metabolite X in diseased cells compared to control cells. As with the previous embodiment, several genes are not expressed in either the control or the diseased cells and two of the genes are expressed at similar levels. In this embodiment, the genes that code for gene products C, D, E, F, I, and L are not expressed while the genes that code for products K and P are expressed in similar levels (diseased cells compared to control cells). However, the gene that codes for product H, which increases the level of metabolite X, is expressed at lower levels in diseased cells and the gene that codes for product J, which decreases the level of metabolite X, is expressed at higher levels. The endogenous/intracellular levels of metabolite X are predicted to exist at lower levels in diseased cells compared to control cells.

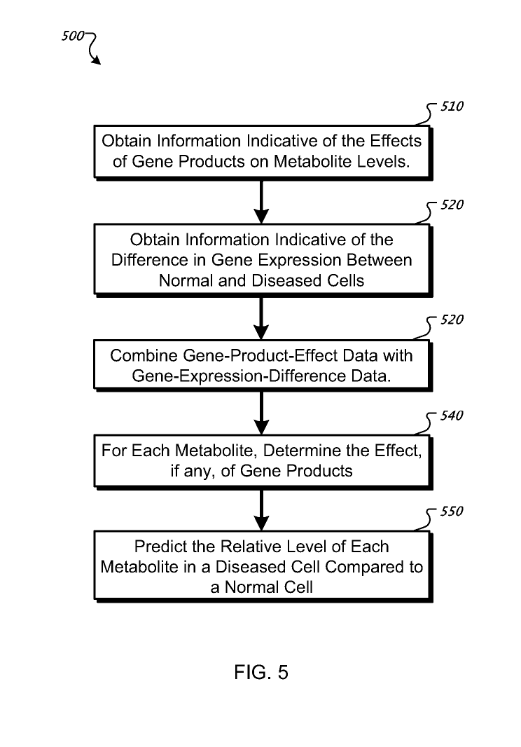

Referring now to FIG. 5, a process 500 can be performed by the metabolomics-based system to predict the relative concentrations of metabolites in diseased cells, compared to the levels in control cells, which can be used to identify metabolites that are predicted to exist in increased/decreased levels in diseased cells. In some embodiments, the process 500 can be performed by the metabolomics-based system during operation 150 (described in connection with FIG. 1). Referring to the process 500, in operation 510, the system can obtain information indicative of the effects of gene products on metabolite levels. For example, as described previously, relationships between metabolites and gene products can be determined from existing information on biochemical pathways, predictions of enzyme function, and the like. In operation 520, the system can obtain information indicative of the difference in gene expression between diseased and control cells. As described elsewhere herein, this can come from an analysis of gene-expression data obtained using DNA microarray technology. In some embodiments, the metabolomics-based system can get the information obtained during operations 510 and 520 from a genetic-metabolic matrix annotated with differential gene-expression data, such as the one produced during operation 140 (described in connection with FIG. 1). An example of such a matrix is depicted in FIG. 3A.

In some embodiments, the process 500 can perform operation 530 and combine the information indicative of the effects of gene products on metabolic levels, obtained during operation 510, with the information obtained during operation 520 that is indicative of genes that are expressed differently in diseased cells, relative to control cells. The result of this combining can, for example, be a genetic-metabolic matrix annotated with the differential expression status data, such as the matrix depicted in FIG. 3B. In operation 540, the information determined in operation 530 can be used to identify, for each metabolite, the effect, if any, of the known gene products. Referring to the genetic-metabolic matrix depicted in FIG. 3B, for example, it can be determined that metabolite X₀₀₀₄ is consumed by enzyme B and produced by enzyme C. From the same figure, it can also be determined that enzyme B is expressed at a similar level in the diseased cells relative to the control cells, and that enzyme C is not produced in detectable amounts in either the control or diseased cells. As will be discussed in greater detail below, in operation 550 this information can be used to predict the relative level of metabolite in diseased cells relative to control cells.

Exemplary rules, employed by the metabolomics-based system (e.g., during operation 550), for predicting the cumulative effect of differential gene expression on the metabolite levels in a cell can be based on the supposition that lower levels of enzymes catalyzing the production of a metabolite and/or higher levels of enzymes catalyzing the consumption of a metabolite each have the effect of decreasing the level of metabolite found in the cell. Conversely, higher levels of enzymes catalyzing the production of a metabolite and/or lower levels of enzymes catalyzing the consumption of a metabolite each have the predicted effect of increasing the level of metabolite found in the cell. The same can be true for gene products other than enzymes, such as small molecule transporters. Increased levels of transporters that move metabolites out of the intracellular environment tend to decrease intracellular level of these metabolites, while increased levels of transporters that move metabolites into the intracellular environment tend to increase the intracellular levels. Decreasing the latter transporters would have the opposite effect.

In some embodiments, the greater the number and/or percentage of gene products that have similar effects on the level of the metabolite, the greater the confidence in the prediction. For example, assume that metabolite A is produced by four enzymes, all of which show decreased expression in diseased cells and is consumed by three enzymes, all of which show increased expression in diseased cells. Also assume that metabolite B is produced by four enzymes, three of which show decreased expression and one of which shows normal expression in diseased cells and is consumed by three enzymes, all of which show increased expression in diseased cells. Since all seven enzymes (100%) related to metabolite A have the effect of decreasing the level of metabolite A (e.g., there are less enzymes that produce it and more that consume it), the confidence level can be high that metabolite A is present at lower quantities in the diseased cells. Regarding metabolite B, 86% (6 out of 7) of the considered gene products have the effect of decreasing the level of metabolite B. In this example, it may still be predicted that metabolite B is found at lower levels in the diseased cells, but the confidence in that prediction may be lower.

In some embodiments, the metabolomics-based system can perform an operation, such as the operation 550 described in connection with FIG. 5, to apply one or more tests to predict the relative levels of metabolites in diseased cells compared to control cells. For example, a metabolite can be included in a group Mᵤₚ (e.g., predicted to have increased levels in diseased cells) when both of the following two tests are true. First, there is at least one gene encoding for a gene product able to increase the intracellular level of the metabolite whose expression status is Gᵤₚ or Gₛᵢₘᵢₗₐᵣ, there is no gene encoding for a gene product able to increase the intracellular level of the metabolite whose expression status is G_{down} (down-regulated in diseased cells), and there is no gene encoding for a gene product able to decrease the intracellular level of the metabolite whose expression status is Gᵤₚ (up-regulated in diseased cells) or Gₛᵢₘᵢₗₐᵣ (significantly expressed at similar levels in diseased and control cells). Second, either or both of the following apply. There is at least one gene encoding for a gene product able to increase the intracellular level of the metabolite whose expression status is Gᵤₚ (up-regulated in diseased cells) and/or there is at least one gene encoding for a gene product able to decrease the intracellular level of the metabolite whose expression status is G_{down} (down-regulated in diseased cells).

Referring again to FIG. 4A, metabolite X can be predicted to exist at increased levels in diseased cells using the above tests because: there are three genes that code for gene products that increase the intracellular level of metabolite level that are either similarly expressed or expressed at higher levels (only one is needed); all the genes that code for gene products that decrease the intracellular level of metabolite X are either not expressed in both or expressed at lower levels in the diseased cells; and of all the genes that code for gene products that increase the intracellular level of metabolite X, two are not expressed in both, two are similarly expressed in both, and two are expressed at higher levels in the diseased cells (e.g., none are expressed at lower levels). Also, one gene product that produces metabolite X exists at higher levels and one gene product consumes metabolite X exists at lower levels (for the above tests to be true, only one of these is required).

Conversely, a metabolite can be included in a group M_{down} (e.g., predicted to have decreased levels in diseased cells) when both of the following two tests are true. First, there is at least one gene encoding for a gene product able to decrease the intracellular level of the metabolite whose expression status is Gᵤₚ or Gₛᵢₘᵢₗₐᵣ, there is no gene encoding for a gene product able to decrease the intracellular level of the metabolite whose expression status is G_{down} (down-regulated in diseased cells), and there is no gene encoding for a gene product able to increase the intracellular level of the metabolite whose expression status is Gᵤₚ (up-regulated in diseased cells) or Gₛᵢₘᵢₗₐᵣ (significantly expressed at similar levels in diseased and control cells). Second, either or both of the following apply. There is at least one gene encoding for a gene product able to increase the intracellular level of the metabolite whose expression status is G_{down} (down-regulated in diseased cells) and/or there is at least one gene encoding for a gene product able to decrease the intracellular level of the metabolite whose expression status is Gᵤₚ (up-regulated in diseased cells).

Referring again to FIG. 4B, metabolite X can be predicted to exist at decreased levels in diseased cells using the above tests because: there are three genes that code for gene products that decrease the intracellular level of metabolite level that are either similarly expressed or expressed at higher levels (only one is needed); all the genes that code for gene products that increase the intracellular level of metabolite X are either not expressed in both or expressed at lower levels in the diseased cells; and of all the genes that code for gene products that decrease the intracellular level of metabolite X, two are not expressed in both, two are similarly expressed in both, and one is expressed at higher levels in the diseased cells (e.g., none are expressed at lower levels). Also, one gene product that produces metabolite X exists at lower levels and one gene product consumes metabolite X exists at higher levels (for the above tests to be true, only one of these is required).

All remaining considered metabolites, which are not assigned a status of Mᵤₚ or M_{down}, can be included in group M_{unknown}, indicating that there is currently no prediction as to whether the level of the metabolite in the cell is increased or decreased in diseased cells, relative to control cells. In this way, the methodology attempts to consider, as much as is practical, the entire proteome complement of enzymes that produce and consume a metabolite.

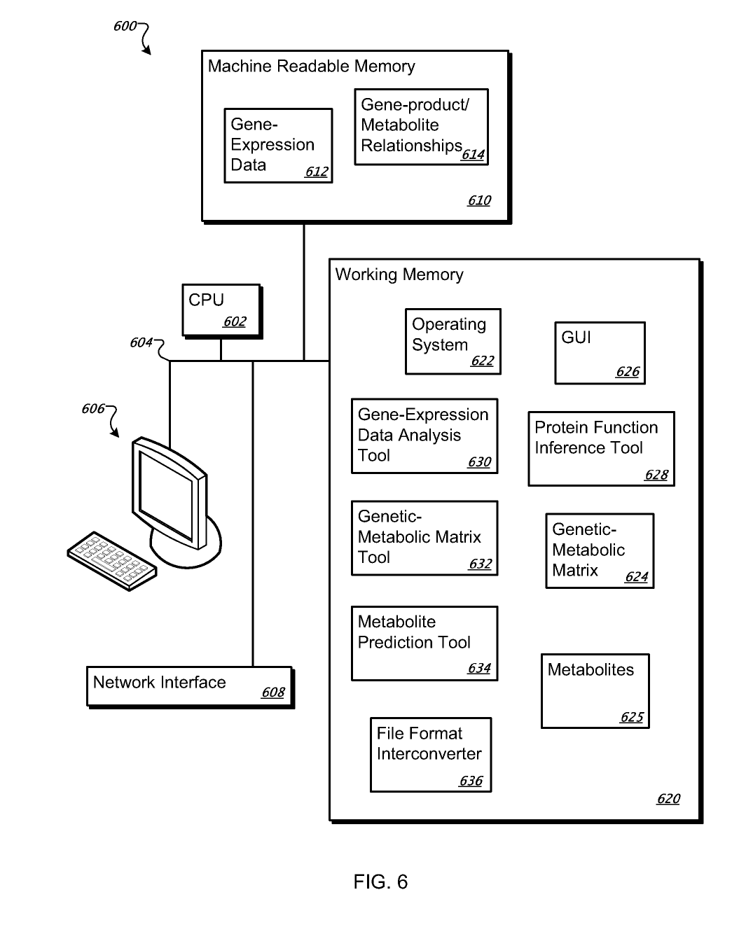

In some embodiments, the metabolites included in the groups Mᵤₚ and M_{down} can be further screened for use in therapeutic treatments. For example, supplementation of a particular metabolite (e.g., one determined to be included in group M_{down}) to raise the intracellular level to a normal physiological level may be of therapeutic value. For certain compounds that are lowered in cancer cells, restoration to levels closer to normal could be achieved by directly administering the deficient metabolite. On the other hand, for metabolites whose levels are increased in cancer cells, reversion to normal levels could involve activation or inhibition of key enzymes. In either case, the approach described herein can identify likely agents and/or targets. In some embodiments, the gene-expression data, the relationships between gene-products and metabolites, the genetic-metabolic matrices, the expression status of one or more genes, and/or metabolites that have potential as agents and/or targets can be stored in electronic form on a computer-readable medium for use with a computer. Additionally, the metabolomics-based methods for identifying potential agents and/or targets for further research can be performed on one or more computers, as depicted in FIG. 6.

Referring now to FIG. 6, a computer system 600 on which metabolomics-based methods as described herein may be carried out can include one or more central processing units 602 for processing machine readable data coupled via a bus 604, to a user interface 606, a network interface 608, a machine readable memory 610, and a working memory 620. The machine readable memory 610 can include a data storage material encoded with machine readable data, wherein the data comprises, for example, gene-expression data 612, and data 614 indicative of relationships between gene-products and metabolites.

Working memory 620 can store an operating system 622, one or more genetic-metabolic matrices 624, and/or one or more metabolites 625 that may be potential agents and/or targets for therapeutic treatment. The computer system 600 can also include a graphical user interface 626 and instructions for processing machine readable data including one or more protein function inference tools 628, one or more gene-expression data analysis tools 630, one or more genetic-metabolic matrix tools 632, one or more metabolite prediction tools 634, and one or more file format interconverters 636.

The computer system 600 may be any of the varieties of laptop or desktop personal computer, or workstation, or a networked or mainframe computer or super-computer, which would be available to one of ordinary skill in the art. For example, computer system 600 may be an IBM-compatible personal computer, a Silicon Graphics, Hewlett-Packard, Fujitsu, NEC, Sun or DEC workstation, or may be a supercomputer of the type formerly popular in academic computing environments. Computer system 600 may also support multiple processors as, for example, in a Silicon Graphics “Origin” system, or a cluster of connected processors.

The operating system 622 may be any suitable variety that runs on any of computer systems 600. For example, in one embodiment, operating system 622 is selected from the UNIX family of operating systems, for example, Ultrix from DEC, AIX from IBM, or IRIX from Silicon Graphics. It may also be a LINUX operating system. In other embodiments, operating system 622 may be a VAX VMS system. In still other embodiments, the operating system 622 can be a DOS operating system or a Windows operating system, such as Windows 3.1, Windows NT, Windows 95, Windows 98, Windows 2000, Windows XP, or Windows Vista. In yet other embodiments, operating system 622 is a Macintosh operating system such as MacOS 7.5.x, MacOS 8.0, MacOS 8.1, MacOS 8.5, MacOS 8.6, MacOS 9.x and MacOS X.

The graphical user interface (“GUI”) 626 is preferably used for displaying genetic-metabolic matrices (e.g., the genetic-metabolic matrix 624), and/or listing metabolites that are potential agents and/or targets for therapeutic treatments, on user interface 606. User-interface 606 may comprise input and output devices such as a keyboard, mouse, touch-screen, display screen, trackpad, scanner, printer, or projector.

The network interface 608 may optionally be used to access one or more metabolic databases and/or sets of gene-expression data stored in the memory of one or more other computers. One or more aspects of the metabolomics-based methods described herein may be carried out with commercially available programs which run on, or with computer programs that are developed specially for the purpose and implemented on, computer system 600. Exemplary commercially available programs can include spreadsheet software (e.g., Excel), pathway analysis software (e.g., Ingenuity, Spotfire, or the like), and microarray data processing software (e.g., dChip). Alternatively, the metabolomics-based methods may be performed with one or more stand-alone programs each of which carries out one or more operations of the metabolomics-based system.

### EXAMPLES

### Example 1

In this example, it is shown that the change in concentration of some metabolites that occur in cancer cells could have an active role in the progress of the disease rather than being a side effect of it. The reversion to a metabolic phenotype more similar to the normal state was explored to determine the possible therapeutic value. For certain compounds that are lowered in cancer cells, restoration to levels closer to normal can be achieved by directly administering the deficient metabolite. On the other hand, for metabolites whose levels are increased in cancer cells, reversion could involve, for example, activation or inhibition of key enzymes, an approach that is more difficult to implement. For that reason, it was decided to focus on the former case. It would be ideal to compare the actual intracellular levels of every human metabolite in normal and diseased states to identify those that are lowered in cancer cells. However, direct large-scale biochemical assays are currently unfeasible. Metabolite profiling based on NMR or mass spectrometry techniques, although very powerful, require costly instruments, and are not free of problems and limitations. In silico methods based on linking enzymes to upregulated microarray-detected transcripts and mapping to metabolic pathways have been applied to the qualitative reconstruction of the metabolome of cancer cells and some predictions have been successfully validated by biochemical experiments. Here, the metabolomics-based method was implemented using CoMet, a fully automated and general computational metabolomics approach to predict the human metabolites whose intracellular levels are more likely to be altered in cancer cells, based on methods described herein. CoMet is further described in: A. K. Arakaki, R. Mezencev, N. Bowen, Y. Huang, J. McDonald and J. Skolnick, “Identification of metabolites with anticancer properties by Computational Metabolomics” Molecular Cancer, 2008:7: 57, incorporated herein by reference. The metabolites predicted to be lowered in cancer compared to normal cells were prioritized as potential anticancer agents. The methodology was applied to a leukemia cell line, and several human metabolites were discovered that, either alone or in combination, exhibited various degrees of antiproliferative activity.

Human T-acute lymphoblastic leukemia Jurkat cells procured from ATCC were grown at RPMI-1640 medium (Mediatech) supplemented with 10% FBS (Gibco), 2 mmol/L L-glutamine (Mediatech), 100 IU/mL penicillin, 100 μg/mL streptomycin, and 0.25 μg/mL amphotericin B (all from Mediatech) at 37° C. in the atmosphere of 5% CO₂, 95% air, and 80% relative humidity. The Jurkat cells were allowed to reach 600,000 cells per mL of suspension culture and about 10⁶ cells from two biological replicates were used for the isolation of total cellular RNA.

RNA quality was verified on the Bioanalyzer RNA Pico Chip (Agilent Technologies). Total RNA was extracted from cell lines using Trizol (Invitrogen). Total RNA from the above extractions was processed using the RiboAmp OA or HS kit (Arcturus) in conjunction with the IVT Labeling Kit from Affymetrix, to produce an amplified, biotin-labeled mRNA suitable for hybridizing to GeneChip Probe Arrays (Affymetrix). Labeled mRNA was hybridized to GeneChip Human Genome U133 Plus 2.0 Arrays in the GeneChip Hybridization oven 640, further processed with the GeneChip Fluidics Station 450 and scanned with the GeneChip Scanner. Affymetrix .CEL files were processed using the Affymetrix Expression Console (EC) Software Version 1.1. Files were processed using the default MASS 3′ expression workflow which includes scaling all probes to a target intensity (TGT) of 500. Spiked in report controls used were AFFX-BioB, AFFX-BioC, AFFX-BioDn, and AFFIX-CreX. Affymetrix .CEL files for three normal lymphoblast samples used as a normal reference to compare Jurkat cells expression data were directly retrieved from the Gene Expression Omnibus (samples GSM113678, GSM113802, and GSM113803 of untreated GM1585 1 cells from the Series GSE5040).

One source of biological information was the Kyoto Encyclopedia of Genes and Genomes (KEGG) of Jul. 5, 2007. The enzyme function annotation for human genes was obtained from the KEGG GENES database, the chemical information about human metabolites from the KEGG LIGAND database, and the metabolic pathway data from the KEGG PATHWAY database. The enzyme function annotations from KEGG were implemented with high confidence predictions made by EFICAz, further described in: A. K. Arakaki, W. Tian, and J. Skolnick, “High accuracy multi-genome scale reannotation of enzyme function by EFICAz” BMC Genomics 2006:7: 315, an approach for enzyme function inference that significantly increased annotation coverage. For the mapping between microarray probe identifiers and Entrez GeneID identifiers, the Affymetrix HG-U133 Plus 2.0 NetAffx Annotation file of May 31, 2007 was used.

The first step in the methodology for the identification of metabolites with anticancer activity consisted of the classification of each enzyme-coding human gene into four possible groups: Gᵤₚ: (upregulated in cancer cells), G_{down}: (downregulated in cancer cells), Gₛᵢₘᵢₗₐᵣ: (expressed in both, normal and cancer cells, at levels that are statistically indistinguishable), and Gₙₒₙₑ: (not expressed in both, normal and cancer cells). Two types of data were used for the classification: the log base 2 signal intensities and the presence calls of the corresponding probe sets, as reported by the Affymetrix Microarray Suite Software 5.0 (MAS 5.0). First, an “off” status was provisionally assigned to each gene in each of the two studied conditions (normal and cancer) if the mean fraction of presence calls labeled as “marginal” or “absent” in the corresponding probe sets is at least 80%, otherwise an “on” status is assigned. Then, each gene was temporarily classified into the Gᵤₚ, G_{down}, Gₛᵢₘᵢₗₐᵣ, or Gₙₒₙₑ group, according to its on/off status in normal and cancer conditions. Finally, genes in the temporary Gₛᵢₘᵢₗₐᵣ or Gₙₒₙₑ groups were transferred to the Gᵤₚ or G_{down} groups if they fulfilled the following criterion for differential expression: the signal intensities in normal and cancer samples exhibited a statistically significant difference in at least 40% of the corresponding probe sets, as evaluated by an ANOVA two tailed test with P<0.005.

The second step in the methodology was an in silico estimation of the effect that the differentially expressed enzyme-encoding genes could have exerted on the intracellular levels of metabolites. First, all the human metabolic pathways were retrieved from the KEGG PATHWAY database, a compilation of maps representing the molecular interactions and reaction networks for different types of biological processes. For the biological process labeled as Metabolism there were eleven groups of pathways: 1) Carbohydrate Metabolism, 2) Energy Metabolism, 3) Lipid Metabolism, 4) Nucleotide Metabolism, 5) Amino Acid Metabolism, 6) Metabolism of Other Amino Acids, 7) Glycan Biosynthesis and Metabolism, 8) Biosynthesis of Polyketides and Nonribosomal Peptides, 9) Metabolism of Cofactors and Vitamins, 10) Biosynthesis of Secondary Metabolites, and 11) Xenobiotics Biodegradation and Metabolism. The pathway maps were available as graphical images and also as KEGG Markup Language (KGML) files that facilitates the parsing of relevant biological data. Thus, the biochemical reactions were extracted from the KGML human metabolic pathway maps, including information about substrates, products, direction/reversibility, and associated enzyme-coding genes.

This information was combined with gene-expression data from normal and cancer cells to construct a genetic-metabolic matrix that linked each of 1,477 metabolites with the specific human genes encoding for enzymes that consume and/or produce each metabolite. The differential expression status given by the four-group classification described in the previous section was stored for each gene. The following were excluded from the genetic-metabolic matrix: i) 209 non-physiological metabolites, here defined as those that only participate in reactions that belong to the “Biosynthesis of Secondary Metabolites” and the “Xenobiotics Biodegradation and Metabolism” groups of metabolic pathways, e.g., ecgonine or parathion, ii) 197 metabolites that are considered ubiquitous and often carry out generic roles in many reactions, here defined as those that are involved as substrate or product in ten or more reactions, e.g., H₂O, ATP, NAD(+)(P) or O₂, and iii) 289 metabolites that participate in reactions that are mainly catalyzed by orphan human enzymes. To determine metabolites belonging to the third category, the number of reactions where a metabolite m acts as substrate or product in human metabolic pathways was defined as Nr_{m,human}, and in reference (non organism specific) metabolic pathways was defined as Nr_{m,ref}. If Nr_{m,human}/Nr_{m,ref}<0.5, then the metabolite m was included in the third exclusion category. The absent reactions in human pathways may be due to orphan enzymes, reactions that only occur in other organisms or reactions that may occur in humans but have not yet been detected, for example, the metabolite 1-alkyl-sn-glycero-3-phosphate was excluded because out of four enzymes that use it as substrate or product, two, EC 2.3.1.105, and EC 1.1.1.101, are orphans in human, and one, EC 2.7.1.93, has only been found in rabbit. The total number of metabolites remaining in the genetic-metabolic matrix after the three types of exclusions was 982.

In this example, a set of rules was used to scan the genetic-metabolic matrix for metabolites whose intracellular levels in cancer cells are likely to differ from those in normal cells. The rules were based on the supposition that lower levels of enzymes catalyzing the production of a metabolite and transporters moving the metabolite into the intracellular space (and/or higher levels of enzymes catalyzing the consumption of the metabolite and transporters moving the metabolite out of the intracellular space) imply a decreased level of such metabolite, and vice versa (see FIGS. 4A and 4B).

In the methodology, a given metabolite was predicted to have decreased levels in cancer cells when: 1) both of the following applied: 1.1) there was no gene encoding for an enzyme able to catalyze the production of the metabolite whose differential expression status was Gᵤₚ (upregulated in cancer cells) or Gₛᵢₘᵢₗₐᵣ (significantly expressed at similar levels in normal and cancer cells) and 1.2) there was no gene encoding for an enzyme able to catalyze the consumption of the metabolite whose differential expression status was G_{down} (downregulated in cancer cells), and 2) either or both of the following applied: 2.1) there was at least one gene encoding for an enzyme able to catalyze the production of the metabolite whose differential expression status was G_{down} (downregulated in cancer cells) and 2.2) there was at least one gene encoding for an enzyme able to catalyze the consumption of the metabolite whose differential expression status was Gᵤₚ (upregulated in cancer cells). Similarly, a metabolite was predicted to have increased levels in cancer cells when: 1) both of the following applies: 1.1) there was no gene encoding for an enzyme able to catalyze the consumption of the metabolite whose differential expression status was Gᵤₚ or Gₛᵢₘᵢₗₐᵣ and 1.2) there was no gene encoding for an enzyme able to catalyze the production of the metabolite whose differential expression status was G_{down}, and 2) either or both of the following applies: 2.1) there was at least one gene encoding for an enzyme able to catalyze the consumption of the metabolite whose differential expression status was G_{down} and 2.2) there was at least one gene encoding for an enzyme able to catalyze the production of the metabolite whose differential expression status was Gᵤₚ.

The in silico metabolomics methods described herein were used to compare two Jurkat cell samples to three normal GM15851 lymphoblast cell samples, which resulted in 104 metabolites predicted to be lowered in the cancer cells (TABLE 1) and 78 metabolites predicted to be increased in the cancer cells (TABLE 2), out of 982 metabolites considered in the analysis (TABLE 4). A search of the literature for experimental evidence identified that 13 of the 982 analyzed metabolites exhibit anticancer activity in Jurkat cells. TABLE 3 shows that 2 of the 13 metabolites were predicted to be lowered in Jurkat cells: thymidine, an antineoplastic agent, and prostaglandin D2, which induces apoptosis without inhibiting the viability of normal T lymphocytes). Only 1 of the 13 proven anticancer agents in Jurkat cells belonged to the group of 78 metabolites predicted to be increased in these cancer cells: the apoptotic agent 2-methoxy-estradiol-17β. The remaining 10 known anticancer molecules active in Jurkat cells: testosterone, melatonin, sphingolipid GD3,2′-deoxyguanosine, 2′-deoxyadenosine, 2′-deoxyinosine, nicotinamide, methylglyoxal, linoleic acid, and cAMP were included in the set of 800 metabolites whose intracellular levels were predicted to be essentially the same in both Jurkat and normal cells. The fraction of metabolites with known anticancer activity among the compounds predicted to be lowered in Jurkat cells (2 of 104 or 0.019) is higher than that corresponding to the rest of the compounds [11 non predicted ones have literature validated anticancer properties; (1+10)/(78+800)=0.013]. However, the significance of this difference cannot be assessed with adequate statistical power due to the small size of the sample. Another complication is the fact that negative results tend to be underreported, thereby making it difficult to obtain unbiased statistics about metabolites that lack anticancer properties.

TABLE 1
METABOLITES WHOSE CONCENTRATION IS PREDICTED TO BE
DECREASED IN JURKAT CELLS COMPARED TO NORMAL
LYMPHOBLASTS

| | KEGG | |
|---|---|---|
| | Ligand | |
| N | identifier | KEGG Ligand description |
| 1 | C00214 | Thymidine; Deoxythymidine |
| 2 | C00255 | Riboflavin; Lactoflavin; 7,8-Dimethyl-10-ribitylisoalloxazine; |
| | | Vitamin B2 |
| 3 | C00299 | Uridine |
| 4 | C00398 | Tryptamine; 3-(2-Aminoethyl)indole |
| 5 | C00447 | D-Sedoheptulose 1,7-bisphosphate; D-altro-Heptulose 1,7- |
| | | biphosphate |
| 6 | C00547 | L-Noradrenaline; Noradrenaline; Norepinephrine; Arterenol; 4- |
| | | [(1R)-2-Amino-1-hydroxyethyl]-1,2-benzenediol |
| 7 | C00606 | 3-Sulfino-L-alanine; L-Cysteinesulfinic acid; 3-Sulphino-L- |
| | | alanine; 3-Sulfinoalanine |
| 8 | C00696 | (5Z,13E)-(15S)-9alpha,15-Dihydroxy-11-oxoprosta-5,13-dienoate; |
| | | Prostaglandin D2 |
| 9 | C00719 | Betaine; Trimethylaminoacetate; Glycine betaine; N,N,N- |
| | | Trimethylglycine; Trimethylammonioacetate |
| 10 | C00762 | Cortisone; 17alpha,21-Dihydroxy-4-pregnene-3,11,20-trione; |
| | | Kendall's compound E; Reichstein's substance Fa |
| 11 | C00788 | L-Adrenaline; (R)-(−)-Adrenaline; (R)-(−)-Epinephrine; (R)-(−)- |
| | | Epirenamine; (R)-(−)-Adnephrine; 4-[(1R)-1-Hydroxy-2- |
| | | (methylamino)ethyl]-1,2-benzenediol |
| 12 | C00828 | Menaquinone; Menatetrenone |
| 13 | C00909 | Leukotriene A4; LTA4; (7E,9E,11Z,14Z)-(5S,6S)-5,6- |
| | | Epoxyeicosa-7,9,11,14-tetraenoic acid; (7E,9E,11Z,14Z)-(5S,6S)- |
| | | 5,6-Epoxyeicosa-7,9,11,14-tetraenoate; (7E,9E,11Z,14Z)-(5S,6S)- |
| | | 5,6-Epoxyicosa-7,9,11,14-tetraenoate |
| 14 | C01026 | N,N-Dimethylglycine; Dimethylglycine |
| 15 | C01036 | 4-Maleylacetoacetate; 4-Maleylacetoacetic acid |
| 16 | C01649 | tRNA(Pro) |
| 17 | C01888 | Aminoacetone; 1-Amino-2-propanone |
| 18 | C02059 | Phylloquinone; Vitamin K1; Phytonadione; 2-Methyl-3-phytyl- |
| | | 1,4-naphthoquinone |
| 19 | C02198 | Thromboxane A2; (5Z,13E)-(15S)-9alpha,11alpha-Epoxy-15- |
| | | hydroxythromboxa-5,13-dienoate; (5Z,9alpha,11alpha,13E,15S)- |
| | | 9,11-Epoxy-15-hydroxythromboxa-5,13-dien-1-oic acid |
| 20 | C02320 | R—S-Glutathione |
| 21 | C02373 | 4-Methylpentanal; Isocaproaldehyde; Isohexanal |
| 22 | C02918 | 1-Methylnicotinamide |
| 23 | C02972 | Dihydrolipoylprotein; [Protein]-dihydrolipoyllysine |
| 24 | C02992 | L-Threonyl-tRNA(Thr) |
| 25 | C03028 | Thiamin triphosphate; Thiamine triphosphate |
| 26 | C03205 | 11-Deoxycorticosterone; Deoxycorticosterone; Cortexone; 21- |
| | | Hydroxy-4-pregnene-3,20-dione; DOC |
| 27 | C03479 | 5-Formyltetrahydrofolate; L(−)-5-Formyl-5,6,7,8-tetrahydrofolic |
| | | acid; Folinic acid |
| 28 | C03512 | L-Tryptophanyl-tRNA(Trp) |
| 29 | C03518 | N-Acetyl-D-glucosaminide |
| 30 | C03546 | myo-Inositol 4-phosphate; D-myo-Inositol 4-phosphate; 1D-myo- |
| | | Inositol 4-phosphate; 1D-myo-Inositol 4-monophosphate; Inositol |
| | | 4-phosphate |
| 31 | C03680 | 4-Imidazolone-5-propanoate; 4-Imidazolone-5-propionic acid; 4,5- |
| | | Dihydro-4-oxo-5-imidazolepropanoate |
| 32 | C03771 | 5-Guanidino-2-oxopentanoate; 5-Guanidino-2-oxo-pentanoate; 2- |
| | | Oxo-5-guanidinopentanoate; 2-Oxo-5-guanidino-pentanoate |
| 33 | C03772 | 5beta-Androstane-3,17-dione |
| 34 | C04006 | 1D-myo-Inositol 3-phosphate; D-myo-Inositol 3-phosphate; myo- |
| | | Inositol 3-phosphate; Inositol 3-phosphate; 1D-myo-Inositol 3- |
| | | monophosphate; D-myo-Inositol 3-monophosphate; myo-Inositol |
| | | 3-monophosphate; Inositol 3-monophosphate; 1L-myo-Inositol 1- |
| | | phosphate; L-myo-Inositol 1-phosphate |
| 35 | C04281 | L-1-Pyrroline-3-hydroxy-5-carboxylate; 3-Hydroxy-L-1-pyrroline- |
| | | 5-carboxylate |
| 36 | C04282 | 1-Pyrroline-4-hydroxy-2-carboxylate |
| 37 | C04409 | 2-Amino-3-carboxymuconate semialdehyde; 2-Amino-3-(3- |
| | | oxoprop-1-enyl)-but-2-enedioate; 2-Amino-3-(3-oxoprop-1-en-1- |
| | | yl)but-2-enedioate |
| 38 | C04438 | 1-Acyl-sn-glycero-3-phosphoethanolamine; L-2- |
| | | Lysophosphatidylethanolamine |
| 39 | C04555 | 3beta-Hydroxyandrost-5-en-17-one 3-sulfate; |
| | | Dehydroepiandrosterone sulfate |
| 40 | C04805 | 5(S)-HETE; 5-Hydroxyeicosatetraenoate; 5-HETE; |
| | | (6E,8Z,11Z,14Z)-(5S)-5-Hydroxyicosa-6,8,11,14-tetraenoic acid |
| 41 | C04853 | 20-OH-Leukotriene B4; 20-OH-LTB4; 20-Hydroxy-leukotriene |
| | | B4; (6Z,8E,10E,14Z)-(5S,12R)-5,12,20-Trihydroxyeicosa- |
| | | 6,8,10,14-tetraenoate; (6Z,8E,10E,14Z)-(5S,12R)-5,12,20- |
| | | Trihydroxyicosa-6,8,10,14-tetraenoate |
| 42 | C05102 | alpha-Hydroxy fatty acid |
| 43 | C05127 | N-Methylhistamine; 1-Methylhistamine; 1-Methyl-4-(2- |
| | | aminoethyl)imidazole |
| 44 | C05235 | Hydroxyacetone; Acetol; 1-Hydroxy-2-propanone; 2-Ketopropyl |
| | | alcohol; Acetone alcohol; Pyruvinalcohol; Pyruvic alcohol; |
| | | Methylketol |
| 45 | C05285 | Adrenosterone |
| 46 | C05290 | 19-Hydroxyandrost-4-ene-3,17-dione; 19- |
| | | Hydroxyandrostenedione |
| 47 | C05293 | 5beta-Dihydrotestosterone |
| 48 | C05294 | 19-Hydroxytestosterone; 17beta,19-Dihydroxyandrost-4-en-3-one |
| 49 | C05332 | Phenethylamine; 2-Phenylethylamine; beta-Phenylethylamine; |
| | | Phenylethylamine |
| 50 | C05335 | Selenomethionine |
| 51 | C05444 | 3alpha,7alpha,26-Trihydroxy-5beta-cholestane; 5beta-Cholestane- |
| | | 3alpha,7alpha,26-triol |
| 52 | C05449 | 3alpha,7alpha-Dihydroxy-5beta-24-oxocholestanoyl-CoA |
| 53 | C05451 | 7alpha-Hydroxy-5beta-cholestan-3-one |
| 54 | C05453 | 7alpha,12alpha-Dihydroxy-5beta-cholestan-3-one |
| 55 | C05473 | 11beta,21-Dihydroxy-3,20-oxo-5beta-pregnan-18-al |
| 56 | C05475 | 11beta,21-Dihydroxy-5beta-pregnane-3,20-dione; 5beta-Pregnane- |
| | | 11beta,21-diol-3,20-dione |
| 57 | C05477 | 21-Hydroxy-5beta-pregnane-3,11,20-trione |
| 58 | C05478 | 3alpha,21-Dihydroxy-5beta-pregnane-11,20-dione; 5beta- |
| | | Pregnane-3alpha,21-diol-11,20-dione |
| 59 | C05479 | 5beta-Pregnane-3,20-dione |
| 60 | C05485 | 21-Hydroxypregnenolone |
| 61 | C05487 | 17alpha,21-Dihydroxypregnenolone |
| 62 | C05488 | 11-Deoxycortisol; Cortodoxone (USAN) |
| 63 | C05503 | Estradiol-17beta 3-glucuronide; 17beta-Estradiol 3-(beta-D- |
| | | glucuronide) |
| 64 | C05504 | 16-Glucuronide-estriol; 16alpha,17beta-Estriol 16-(beta-D- |
| | | glucuronide) |
| 65 | C05585 | Gentisate aldehyde |
| 66 | C05636 | 3-Hydroxykynurenamine |
| 67 | C05638 | 5-Hydroxykynurenamine |
| 68 | C05642 | Formyl-N-acetyl-5-methoxykynurenamine |
| 69 | C05643 | 6-Hydroxymelatonin |
| 70 | C05647 | Formyl-5-hydroxykynurenamine |
| 71 | C05648 | 5-Hydroxy-N-formylkynurenine |
| 72 | C05653 | Formylanthranilate; N-Formylanthranilate; 2-(Formylamino)- |
| | | benzoic acid |
| 73 | C05775 | alpha-Ribazole; N1-(alpha-D-ribosyl)-5,6-dimethylbenzimidazole |
| 74 | C05787 | Bilirubin beta-diglucuronide; Bilirubin-bisglucuronoside |
| 75 | C05796 | Galactan |
| 76 | C05802 | 2-Hexaprenyl-6-methoxyphenol |
| 77 | C05804 | 2-Hexaprenyl-3-methyl-6-methoxy-1,4-benzoquinone |
| 78 | C05814 | 2-Octaprenyl-3-methyl-6-methoxy-1,4-benzoquinone |
| 79 | C05832 | 5-Hydroxyindoleacetylglycine |
| 80 | C05984 | 2-Hydroxybutanoic acid; 2-Hydroxybutyrate; 2-Hydroxybutyric |
| | | acid |
| 81 | C06000 | (S)-3-Hydroxyisobutyryl-CoA |
| 82 | C06056 | 4-Hydroxy-L-threonine |
| 83 | C11131 | 2-Methoxy-estradiol-17beta 3-glucuronide |
| 84 | C11132 | 2-Methoxyestrone 3-glucuronide |
| 85 | C11133 | Estrone glucuronide; Estrone 3-glucuronide; Estrone beta-D- |
| | | glucuronide |
| 86 | C11134 | Testosterone glucuronide; Testosterone 17beta-(beta-D- |
| | | glucuronide) |
| 87 | C11135 | Androsterone glucuronide; Androsterone 3-glucuronide |
| 88 | C11136 | Etiocholan-3alpha-ol-17-one 3-glucuronide |
| 89 | C11508 | 4alpha-Methyl-5alpha-ergosta-8,14,24(28)-trien-3beta-ol; |
| | | delta8,14-Sterol |
| 90 | C11521 | UDP-6-sulfoquinovose |
| 91 | C14765 | 13-OxoODE; 13-KODE; (9Z,11E)-13-Oxooctadeca-9,11-dienoic |
| | | acid |
| 92 | C14782 | 11,12,15-THETA; 11,12,15-Trihydroxyicosatrienoic acid; |
| | | (5Z,8Z,13E)-(15S)-11,12,15-Trihydroxyeicosa-5,8,12-trienoic |
| | | acid; (5Z,8Z,13E)-(15S)-11,12,15-Trihydroxyicosa-5,8,12-trienoic |
| | | acid |
| 93 | C14814 | 11,14,15-THETA; 11,14,15-Trihydroxyicosatrienoic acid; |
| | | (5Z,8Z,12E)-11,14,15-Trihydroxyeicosa-5,8,12-trienoic acid; |
| | | (5Z,8Z,12E)-11,14,15-Trihydroxyicosa-5,8,12-trienoic acid |
| 94 | C14819 | Fe3+; Fe(III); Ferric ion; Iron(3+) |
| 95 | C14827 | 9(S)-HPODE; 9(S)-HPOD; (10E,12Z)-(9S)-9- |
| | | Hydroperoxyoctadeca-10,12-dienoic acid |
| 96 | C15780 | 5-Dehydroepisterol |
| 97 | C15783 | 5-Dehydroavenasterol |
| 98 | G00025 | (Gal)1 (GalNAc)1 (GlcNAc)1 (Ser/Thr)1; Glycoprotein; O-Glycan |
| 99 | G00031 | (GalNAc)1 (GlcNAc)1 (Ser/Thr)1; Glycoprotein; O-Glycan |
| 100 | G00143 | (GlcNAc)1 (Ino-P)1; Glycoprotein; GPI anchor |
| 101 | G00145 | (GlcN)1 (Ino(acyl)-P)1; Glycoprotein; GPI anchor |
| 102 | G00147 | (GlcN)1 (Ino(acyl)-P)1 (Man)1 (EtN)1 (P)1; Glycoprotein; GPI |
| | | anchor |
| 103 | G10611 | UDP-N-acetyl-D-galactosamine; UDP-N-acetylgalactosamine; |
| | | (UDP-GalNAc)1 |
| 104 | G10617 | Dolichyl phosphate D-mannose; Dolichyl D-mannosyl phosphate; |
| | | (Man)1 (P-Dol)1 |

TABLE 2
METABOLITES WHOSE CONCENTRATION IS PREDICTED TO BE
INCREASED IN JURKAT CELLS COMPARED TO NORMAL LYMPHOBLASTS

| | KEGG | |
|---|---|---|
| | Ligand | |
| N | identifier | KEGG Ligand description |
| 1 | C00012 | Peptide |
| 2 | C00410 | Progesterone; 4-Pregnene-3,20-dione |
| 3 | C00439 | N-Formimino-L-glutamate; N-Formimidoyl-L-glutamate |
| 4 | C00461 | Chitin; beta-1,4-Poly-N-acetyl-D-glucosamine; [1,4-(N-Acetyl-beta-D- |
| | | glucosaminyl)]n; [1,4-(N-Acetyl-beta-D-glucosaminyl]n + 1 |
| 5 | C00486 | Bilirubin |
| 6 | C00523 | Androsterone; 3alpha-Hydroxy-5alpha-androstan-17-one |
| 7 | C00584 | Prostaglandin E2; (5Z,13E)-(15S)-11alpha,15-Dihydroxy-9- |
| | | oxoprosta-5,13-dienoate; (5Z,13E)-(15S)-11alpha,15-Dihydroxy-9- |
| | | oxoprost-13-enoate; Dinoprostone |
| 8 | C00643 | 5-Hydroxy-L-tryptophan |
| 9 | C01042 | N-Acetyl-L-aspartate |
| 10 | C01044 | N-Formyl-L-aspartate |
| 11 | C01102 | O-Phospho-L-homoserine |
| 12 | C01143 | (R)-5-Diphosphomevalonate |
| 13 | C01322 | RX |
| 14 | C01353 | Carbonic acid; Dihydrogen carbonate; H2CO3 |
| 15 | C01598 | Melatonin; N-Acetyl-5-methoxytryptamine |
| 16 | C01651 | tRNA(Thr) |
| 17 | C01652 | tRNA(Trp) |
| 18 | C01708 | Hemoglobin |
| 19 | C01780 | Aldosterone; 11beta,21-Dihydroxy-3,20-dioxo-4-pregnen-18-al |
| 20 | C01798 | D-Glucoside |
| 21 | C01921 | Glycocholate; Glycocholic acid; 3alpha,7alpha,12alpha- |
| | | Trihydroxy-5beta-cholan-24-oylglycine |
| 22 | C01943 | Obtusifoliol; 4alpha,14alpha-Dimethyl-5alpha-ergosta-8,24(28)- |
| | | dien-3beta-ol; 4alpha,14alpha-Dimethyl-24-methylene-5alpha- |
| | | cholesta-8-en-3beta-ol |
| 23 | C02051 | Lipoylprotein; H-Protein-lipoyllysine |
| 24 | C02165 | Leukotriene B4; (6Z,8E,10E,14Z)-(5S,12R)-5,12-Dihydroxyeicosa- |
| | | 6,8,10,14-tetraenoate; (6Z,8E,10E,14Z)-(5S,12R)-5,12- |
| | | Dihydroxyicosa-6,8,10,14-tetraenoate |
| 25 | C02218 | 2-Aminoacrylate; Dehydroalanine |
| 26 | C02702 | L-Prolyl-tRNA(Pro) |
| 27 | C03267 | beta-D-Fructose 2-phosphate; beta-D-Fructofuranose 2-phosphate |
| 28 | C03547 | omega-Hydroxy fatty acid |
| 29 | C04373 | 3alpha-Hydroxy-5beta-androstan-17-one; Etiocholan-3alpha-ol-17- |
| | | one; 3alpha-Hydroxyetiocholan-17-one |
| 30 | C04454 | 5-Amino-6-(5′-phosphoribitylamino)uracil; 5-Amino-2,6-dioxy-4- |
| | | (5′-phosphoribitylamino)pyrimidine; 5-Amino-6-(5- |
| | | phosphoribitylamino)uracil |
| 31 | C04778 | N1-(5-Phospho-alpha-D-ribosyl)-5,6-dimethylbenzimidazole; |
| | | alpha-Ribazole 5′-phosphate |
| 32 | C04874 | 2-Amino-4-hydroxy-6-(D-erythro-1,2,3-trihydroxypropyl)-7,8- |
| | | dihydropteridine; Dihydroneopterin |
| 33 | C05122 | Taurocholate; Taurocholic acid; Cholyltaurine |
| 34 | C05212 | 1-Radyl-2-acyl-sn-glycero-3-phosphocholine; 1-Organyl-2-acyl-sn- |
| | | glycero-3-phosphocholine; 2-Acyl-1-alkyl-sn-glycero-3- |
| | | phosphocholine |
| 35 | C05284 | 11beta-Hydroxyandrost-4-ene-3,17-dione; Androst-4-ene-3,17- |
| | | dione-11beta-ol; 4-Androsten-11beta-ol-3,17-dione |
| 36 | C05299 | 2-Methoxyestrone |
| 37 | C05302 | 2-Methoxyestradiol-17beta |
| 38 | C05448 | 3alpha,7alpha,24-Trihydroxy-5beta-cholestanoyl-CoA |
| 39 | C05462 | Chenodeoxyglycocholate |
| 40 | C05476 | Tetrahydrocorticosterone |
| 41 | C05498 | 11beta-Hydroxyprogesterone |
| 42 | C05527 | 3-Sulfinylpyruvate; 3-Sulfinopyruvate |
| 43 | C05546 | Protein N6,N6,N6-trimethyl-L-lysine |
| 44 | C05582 | Homovanillate; Homovanillic acid |
| 45 | C05584 | 3-Methoxy-4-hydroxymandelate; Vanillylmandelic acid |
| 46 | C05635 | 5-Hydroxyindoleacetate |
| 47 | C05637 | 4,8-Dihydroxyquinoline; Quinoline-4,8-diol |
| 48 | C05639 | 4,6-Dihydroxyquinoline; Quinoline-4,6-diol |
| 49 | C05713 | Cyanoglycoside |
| 50 | C05803 | 2-Hexaprenyl-6-methoxy-1,4-benzoquinone |
| 51 | C05813 | 2-Octaprenyl-6-methoxy-1,4-benzoquinone |
| 52 | C05823 | 3-Mercaptolactate |
| 53 | C05828 | Methylimidazoleacetic acid; Tele-methylimidazoleacetic acid; 1- |
| | | Methyl-4-imidazoleacetic acid; 1-Methylimidazole-4-acetate; |
| | | Methylimidazoleacetate |
| 54 | C05842 | N1-Methyl-2-pyridone-5-carboxamide; N′-Methyl-2-pyridone-5- |
| | | carboxamide |
| 55 | C05843 | N1-Methyl-4-pyridone-5-carboxamide; N′-Methyl-4-pyridone-5- |
| | | carboxamide |
| 56 | C06125 | Sulfatide; Galactosylceramidesulfate; Cerebroside 3-sulfate |
| 57 | C06197 | P1,P3-Bis(5′-adenosyl) triphosphate; ApppA |
| 58 | C06426 | (6Z,9Z,12Z)-Octadecatrienoic acid; 6,9,12-Octadecatrienoic acid; |
| | | gamma-Linolenic acid |
| 59 | C11554 | 1-Phosphatidyl-1D-myo-inositol 3,4-bisphosphate; 1,2-Diacyl-sn- |
| | | glycero-3-phospho-(1′-myo-inositol-3′,4′-bisphosphate) |
| 60 | C13309 | 2-Phytyl-1,4-naphthoquinone; Demethylphylloquinone |
| 61 | C13508 | Sulfoquinovosyldiacylglycerol; SQDG; 1,2-Diacyl-3-(6-sulfo- |
| | | alpha-D-quinovosyl)-sn-glycerol |
| 62 | C14762 | 13(S)-HODE; (13S)-Hydroxyoctadecadienoic acid; (9Z,11E)-(13S)- |
| | | 13-Hydroxyoctadeca-9,11-dienoic acid |
| 63 | C14772 | 5,6-DHET; (8Z,11Z,14Z)-5,6-Dihydroxyeicosa-8,11,14-trienoic |
| | | acid; (8Z,11Z,14Z)-5,6-Dihydroxyicosa-8,11,14-trienoic acid |
| 64 | C14773 | 8,9-DHET; (5Z,11Z,14Z)-8,9-Dihydroxyeicosa-5,11,14-trienoic |
| | | acid; (5Z,11Z,14Z)-8,9-Dihydroxyicosa-5,11,14-trienoic acid |
| 65 | C14774 | 11,12-DHET; (5Z,8Z,14Z)-11,12-Dihydroxyeicosa-5,8,14-trienoic |
| | | acid; (5Z,8Z,14Z)-11,12-Dihydroxyicosa-5,8,14-trienoic acid |
| 66 | C14775 | 14,15-DHET; (5Z,8Z,11Z)-14,15-Dihydroxyeicosa-5,8,11-trienoic |
| | | acid; (5Z,8Z,11Z)-14,15-Dihydroxyicosa-5,8,11-trienoic acid |
| 67 | C14778 | 16(R)-HETE; (5Z,8Z,11Z,14Z)-(16R)-16-Hydroxyeicosa- |
| | | 5,8,11,14-tetraenoic acid; (5Z,8Z,11Z,14Z)-(16R)-16- |
| | | Hydroxyicosa-5,8,11,14-tetraenoic acid |
| 68 | C14781 | 15H-11,12-EETA; 15-Hydroxy-11,12-epoxyeicosatrienoic acid; |
| | | (5Z,8Z,13E)-(15S)-11,12-Epoxy-15-hydroxyeicosa-5,8,13-trienoic |
| | | acid; (5Z,8Z,13E)-(15S)-11,12-Epoxy-15-hydroxyicosa-5,8,13- |
| | | trienoic acid |
| 69 | C14813 | 11H-14,15-EETA; 11-Hydroxy-14,15-EETA; 11-Hydroxy-14,15- |
| | | epoxyeicosatrienoic acid; (5Z,8Z,12E)-14,15-Epoxy-11- |
| | | hydroxyeicosa-5,8,12-trienoic acid; (5Z,8Z,12E)-14,15-Epoxy-11- |
| | | hydroxyicosa-5,8,12-trienoic acid |
| 70 | C14825 | 9(10)-EpOME; (9R,10S)-(12Z)-9,10-Epoxyoctadecenoic acid |
| 71 | C14826 | 12(13)-EpOME; (12R,13S)-(9Z)-12,13-Epoxyoctadecenoic acid |
| 72 | C15647 | 2-Acyl-1-(1-alkenyl)-sn-glycero-3-phosphate |
| 73 | C15782 | delta7-Avenasterol |
| 74 | G00032 | (Gal)1 (GalNAc)1 (GlcNAc)1 (Ser/Thr)1; Glycoprotein; O-Glycan |
| 75 | G00038 | (Gal)3 (Glc)1 (GlcNAc)1 (Cer)1; Glycolipid; Sphingolipid |
| 76 | G00140 | (GlcN)1 (Ino(acyl)-P)1 (Man)4 (EtN)1 (P)1; Glycoprotein; GPI |
| | | anchor |
| 77 | G00146 | (GlcN)1 (Ino(acyl)-P)1 (Man)1; Glycoprotein; GPI anchor |
| 78 | G12396 | 6-(alpha-D-glucosaminyl)-1D-myo-inositol; (GlcN)1 (Ino)1 |

The ligand descriptors in the third column of Table 2 include generic descriptors that refer to classes of molecules, e.g., a peptide. Many of the most general descriptors are discarded from subsequent analyses.

Based on criteria such as low molecular weight, commercial availability, and affordability, nine metabolites predicted to be lowered in Jurkat cells were selected to test their effect on the proliferation of that cell line (TABLE 3). The effect of a 72 hour treatment on the growth of Jurkat cells was examined using the following metabolites (at a concentration of 100 μM): riboflavin, tryptamine, 3-sulfino-L-alanine, menaquinone, dehydroepiandrosterone (the non-sulfated version of the predicted metabolite dehydroepiandrosterone sulfate), α-hydroxystearic acid (one of the possible compounds compatible with the predicted generic metabolite α-hydroxy fatty acid), hydroxyacetone, seleno-L-methionine, and 5,6-dimethylbenzimidazole (the aglycone of the predicted metabolite a-ribazole).

TABLE 3
Active metabolites predicted to be lowered in Jurkat cells

Previously known anticancer activity in Jurkat cells
thymidine (C00214)¹
prostaglandin D2 (C00696)
Anticancer activity in Jurkat cells tested in this work
riboflavin (C00255)
tryptamine (C00398)
3-sulfino-L-alanine (C00606)
menaquinone (C00828)
dehydroepiandrosterone sulfate (C04555)
α-hydroxy fatty acid (C05102)
hydroxyacetone (C05235)
seleno-L-methionine (C05335)
α-ribazole (C05775)
¹KEGG ligand identifier

Growth inhibition of Jurkat cells was evaluated by a resazurin-based in vitro toxicology assay kit (Sigma). Metabolites dehydroepiandrosterone (dehydroisoandrosterone, Acros Organics), 5,6-diqnethylbenzimidazole (Aldrich), hydroxyacetone (Sigma), menaquinone (Supelco), riboflavin (Sigma) and tryptamine (Sigma) were solubilized in DMSO (Sigma); 3-sulfino-L-alanine (L-cysteinesulfinic acid, Aldrich) and seleno-L-methionine (Sigma) were solubilized in sterile deionized water and stock solutions (40 mmol/L) were stored frozen at −80° C. prior to its use. Aliquots of 100 μL of cells in phenol red free RPMI 1640 medium (Sigma) supplemented with 5% FBS, 2 mmol/L L-glutamine, 100 IU/mL penicillin, 100 μL/mL streptomycin, and 0.25 μL/mL amphotericin B were inoculated into 96-well black-walled plates at a density of 250,000 cells/mL (Jurkat) or 200,000 cells/mL (OVCAR-3) and incubated for 24 hours at 37° C. in 5% CO₂, 95% air, and 80% relative humidity prior to the addition of the metabolites to be tested. Stock solutions of metabolites were diluted 200 times with complete growth medium and added to the appropriate microliter wells in 4 replicates per metabolite, while 100 μL of complete medium was added to the control and blank cells. Following metabolite addition, the plates were incubated far an additional 72 hours, after which 20 μL of TOX-8 reagent was added to metabolite treatment, control and blank wells and incubation continued for additional 3 hours. The increase in fluorescence was measured in a microplate fluorimeter at 590 nm using an excitation wavelength of 560 nm. The emission of control wells, after the subtraction of a blank, was taken as 100% and the results for metabolite treatments were expressed as percentage of the control. Two biological replicates for each cell line were used for cell proliferation assays. Positive results were additionally verified by counting of viable cells using Vi-CELL XR cell counter (Beckman Coulter) and trypan blue dye exclusion method for Jurkat.

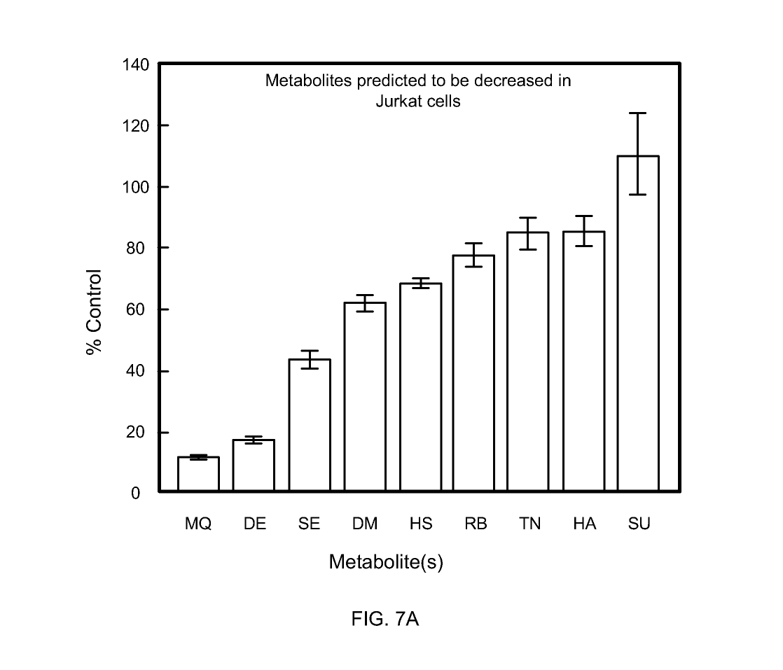

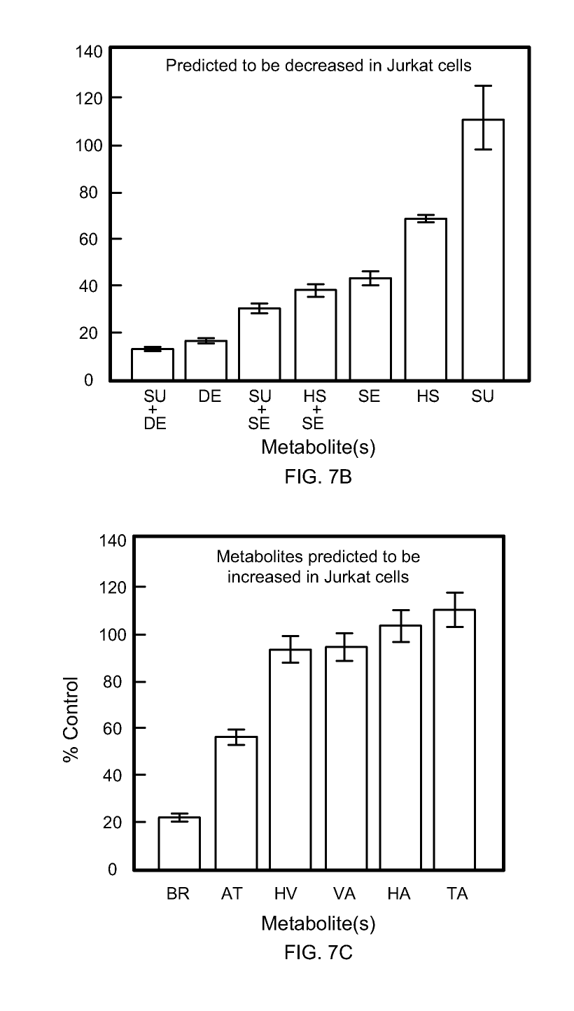

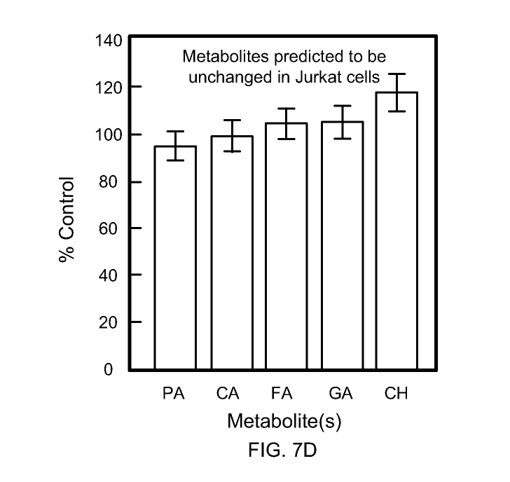

FIG. 7A shows that eight out of the nine metabolites predicted to be lowered in Jurkat cells (with the exception of sulfino-L-alanine) exhibited an inhibition of Jurkat cell growth below 90% of the untreated control (as evaluated by two-tailed t-tests at a critical alpha level of 0.05). As shown in FIG. 7B, although sulfino-L-alanine alone did not inhibit the growth of Jurkat cells, it significantly potentiated the inhibitory effect of seleno-L-methionine from 43.1% to 30.3% and slightly potentiated the inhibitory activity of dehydroepiandrosterone from 16.7% to 13.6%. Similarly, a synergistic interaction between 5,6-di-ethylbenzimidazole (61.4%) and seleno-L-methionine lead to a supra-additive inhibitory activity of 19.2%. The synergistic effect displayed by these metabolites indicates that a strategy able to prioritize specific combinations of metabolites whose anticancer effect should be simultaneously tested may lead to the discovery of treatments of increased efficacy. On the other hand, α-hydroxystearic acid (67.8%) and dehydroepiandrosterone showed an additive effect, while α-hydroxystearic acid and seleno-L-methionine exhibited a sub-additive or antagonistic inhibitory activity of 37.7%. Menaquinone (FIG. 7A) showed the highest antiproliferative activity (11.3%), whereas the inhibitory activity of riboflavin, tryptamine, and hydroxyacetone on Jurkat cells was more moderate, all above 70%.

Although the fact that the nine tested metabolites predicted to be lowered in Jurkat cells exhibited antiproliferative activity strongly support our hypothesis, the possibility still exists that most endogenous metabolites inhibit the growth of Jurkat cells, independent of the intracellular level status predicted by the metabolomics-based system described here. Therefore, we tested metabolites whose intracellular levels in Jurkat cells were predicted to be increased (bilirubin, androsterone, homovanillic acid, vanillylmandelic acid, N-acetyl-L-aspartate, and taurocholic acid) or unchanged (pantothenic acid, citric acid, folic acid, P-D-galactose, cholesterol) compared with normal lymphoblasts. We analyzed the effect on the growth of Jurkat cells of a 72 hour treatment with each of the eleven human metabolites at a concentration of 100 μM. FIG. 7C shows that only two of the six tested metabolites whose concentrations are predicted to be increased in Jurkat cells exhibit significant antiproliferative activity: bilirubin (21.3%) and androsterone (54.5%). The growth inhibition exerted by each of the remaining tested metabolites was above 90% and statistically insignificant. Similarly, FIG. 7D shows that all the tested metabolites whose intracellular levels in Jurkat cells and normal lymphoblasts we predict to be comparable, exhibit a statistically insignificant antiproliferative activity above 90%. Statistical significance was evaluated in all the cases according to two-tailed t-tests at a critical alpha level of 0.05.

While the inhibitory activity of riboflavin, tryptamine and hydroxyacetone on Jurkat cells was moderate (all above 70% growth compared to control), others like menaquinone and DHEA exhibited an important inhibitory effect (11.3% and 16.7% growth compared to the control, respectively). Only 2/11 tested metabolites predicted not to be lowered in Jurkat cells unexpectedly exhibited antiproliferative activity, while the growth inhibition exerted by each of the remaining tested metabolites was less than 10% and statistically insignificant (FIGS. 6C and 6D). Thus, 18/20 assayed metabolites behave according to the hypothesis regarding the active role of endogenous metabolites in cancer (i.e., that metabolites that have lowered levels in a cancer cell as compared to normal cells might contribute to the progress of the disease).

If the nine novel antiproliferative compounds described herein are considered and the two metabolites whose anticancer activity in Jurkat cells was previously known, the fraction of anticancer metabolites among the 104 compounds predicted to be lowered in Jurkat cells is considerably higher [(9+2)/104=0.106] than that corresponding to the rest of the compounds [(2+11)/878=0.015]. The positive association between lowered metabolite levels in Jurkat cells as predicted by CoMet and antiproliferative activity of the metabolite in that cell line is highly significant (Fisher's exact test two-tailed p-value=8.7×10⁻⁶). Furthermore, when the effect of these metabolites on growth inhibition was tested in Jurkat and human lymphoblast cells cultured in identical conditions, a pattern of selectivity of the antiproliferative effect towards the cancer cell line became evident. In an extreme case, DHEA at a concentration of 50 μM inhibited the growth of Jurkat cells but stimulated the proliferation of lymphoblasts.

### Example 2

Since the results on Jurkat cells were encouraging, a more demanding test was performed in order to evaluate the range of applicability of the in silico metabolomics methods described herein, and the general validity of the correlation between predicted lowered concentration of a metabolite in cancer cells and its anticancer activity. A comparative analysis of the potency of drugs used in current chemotherapy tested on the National Cancer Institute cell lines revealed that leukemia cell lines are the most sensitive ones, while the most resilient cell lines originate from ovarian tissue. Therefore, the OVCAR-3 cell line was chosen to test.

A methodology similar to that of example 1 was used to identify one or more metabolites associated with the OVCAR-3 cell line that may have potential as agents and/or targets for therapeutic treatment. The OVCAR-3 cell line is derived from malignant ascites of a patient with progressive adenocarcinoma of the ovary after failed cisplatin therapy. Gene expression data from three OVCAR-3 cell samples was obtained and compared to expression data from three human immortalized ovarian surface epithelial (IOSE) cell samples (samples GSM154124 and GSM154125 in GEO). Based on this information, CoMet predicted 132 metabolites to be lowered and 120 metabolites to be increased in OVCAR-3 cancer cells. Two of the 132 metabolites predicted to be lowered in OVCAR-3,2-methoxyestradiol and calcitriol, and two of the 730 predicted to be unchanged, 3′,3,5-triiodo-L-thyronine and all-trans-retinoic acid, had previously been demonstrated to exhibit anticancer activity in OVCAR-3 cells.

Growth inhibition of OVCAR-3 cells was evaluated by a resazurin-based in vitro toxicology assay kit (Sigma). Metabolites dehydroepiandrosterone (dehydroisoandrosterone, Acros Organics), 5,6-dignethylbenzimidazole (Aldrich), hydroxyacetone (Sigma), menaquinone (Supelco), riboflavin (Sigma) and tryptarnine (Sigma) were solubilized in DMSO (Sigma); 3-sulfino-L-alanine (L-cysteinesulfinic acid, Aldrich) and seleno-L-methionine (Sigma) were solubilized in sterile deionized water and stock solutions (40 mmol/L) were stored frozen at −80° C. prior to its use. Aliquots of 100 μL of cells in phenol red free RPMI 1640 medium (Sigma) supplemented with 5% FBS, 2 mmol/L L-glutamine, 100 IU/mL penicillin, 100 μL/mL streptomycin, and 0.25 μL/mL amphotericin B were inoculated into 96-well black-walled plates at a density of 200,000 cells/mL and incubated for 24 hours at 37° C. in 5% CO₂, 95% air, and 80% relative humidity prior to the addition of the metabolites to be tested. Stock solutions of metabolites were diluted 200 times with complete growth medium and added to the appropriate microliter wells in 4 replicates per metabolite, while 100 μL of complete medium was added to the control and blank cells. Following metabolite addition, the plates were incubated for an additional 72 hours, after which 20 μL of TOX-8 reagent was added to metabolite treatment, control and blank wells and incubation continued for additional 2 hours. The increase in fluorescence was measured in a microplate fluorimeter at 590 nm using an excitation wavelength of 560 nm. The emission of control wells, after the subtraction of a blank, was taken as 100% and the results for metabolite treatments were expressed as percentage of the control. Two biological replicates for each cell line were used for cell proliferation assays. Positive results were additionally verified by counting of viable cells using Vi-CELL XR cell counter (Beckman Coulter) and SRB-based assay for OVCAR-3 cells.

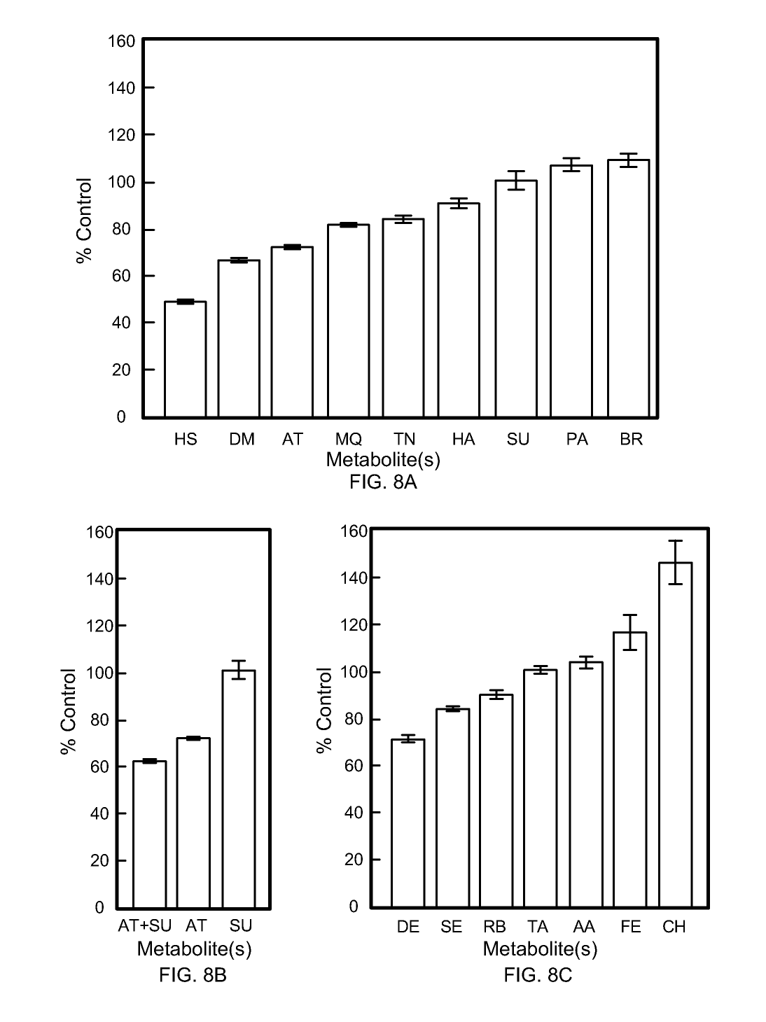

FIG. 8A shows that five of nine tested metabolites predicted to be lowered in OVCAR-3 cells exhibited an inhibition of OVCAR-3 cell growth below 90% of the untreated control (the experimental conditions and statistical analysis are the same as described in example 1 for Jurkat cells). Sulfino-L-alanine exhibited the same behavior as in Jurkat cells (see example 1); although alone it did not inhibit the growth of OVCAR-3 cells, it potentiated the inhibitory effect of androsterone (FIG. 8B). On the other hand, only two of the seven tested metabolites predicted not to be lowered in OVCAR-3 cells showed a significant antiproliferative effect on the cancer cell line (FIG. 8C). The positive association between lowered metabolite levels in OVCAR-3 cells as predicted by CoMet and antiproliferative activity of the metabolite in that cell line is highly significant (Fisher's exact test two-tailed p-value=2.7×10⁻⁵). Thus, the results on Jurkat cells from example 1 and OVCAR-3 cells from example 2 show a similar trend, suggesting that the approach to predict antiproliferative metabolites may have general applicability. Interestingly, the growth inhibitory effect on OVCAR-3 of some of the anticancer metabolites discovered by CoMet is comparable to that of taxol (a drug commonly used against ovarian cancer) in the same cell line.

The growth inhibitory effects of some of the predicted compounds may seem relatively low, and the tested concentration of 100 μmol/L may seem too high, compared with most anticancer drugs of synthetic or natural origin. However, this concentration is not unreasonably high for metabolic compounds, since many metabolites can be found at similar levels in the cytosol and/or extracellular fluids. Also, several of the newly found antiproliferative metabolites exhibited synergistic interactions among them, which is consistent with the systematic approach of the methods in that the prediction was performed on the entire metabolome and not on individual metabolites or pathways. This observation raises the intriguing question of what the result would be if concentrations close to those observed in the normal cells could be achieved in the cancer cell for most of the metabolites, i.e., a reversion to a normal like metabolic profile, at least for those metabolites that exhibit the ability of inhibiting the growth of the cancer cell. In addition, some active metabolites might be considered as completely novel lead compounds for further drug design and development, with the advantage of a reduced initial toxicity.

The mode of action of the newly found antiproliferative metabolites has not been investigated, and it is even possible that some of them may exert their effect based on completely novel mechanisms, however, for most metabolites a possible mode of action based on their effect on other cancer cells or on the known properties of closely related molecules can be suggested. For example, 5,6-dichlorobenzimidazole, a bioisosteric derivative of the active metabolite 5,6-dimethylbenzimidazole, induces differentiation of malignant erythroblasts by inhibiting RNA polymerase II. The tested metabolite tryptamine is an effective inhibitor of HeLa cell growth via the competitive inhibition of tryptophanyl-tRNA synthetase, and consequent inhibition of protein biosynthesis. 9-hydroxystearic acid, an isomer of the active metabolite α-hydroxystearic acid, arrests HT29 colon cancer cells in G0/G1 phase of the cell cycle via overexpression of p21 and induces differentiation of HT29 cells by inhibition of histone deacetylase 1 and interrupts the transduction of the mitogenic signal. Menaquinone (vitamin K2), the most efficient compound among the metabolites tested in Jurkat, has been previously reported to induce G0/G1 arrest, differentiation, and apoptosis in acute myelomonocytic leukemia HL-60 cells. However, considering the great difference between acute lymphoblastic and myelomonocytic leukemias in their etiology, pathogenesis, prognosis, and treatment response, the finding of growth inhibition of Jurkat cells by menaquinone is novel and may even have a different underlying mechanism.

There are several factors not accounted for in the methodology that can influence the actual intracellular levels of a metabolite, and constitute possible sources of error that could affect the predictions. First, the initial input in the methods comes from microarray data, however, the gene expression levels inferred from microarray experiments are subject to several sources of variation due to biological or technical causes.

Second, the analysis depends on the mapping of genes, but this mapping is imperfect because: i) errors have been detected in the gene mappings provided by the microarray manufacturer, ii) not all the genes are represented in a microarray, e.g., only 14,500 human genes are represented in the Affymetrix GeneChip Human Genome U133A 3.0 Array employed herein, although the most conservative estimations indicate that there are at least 18,000 protein-coding genes in the human genome, and iii) alternatively spliced genes can generate catalytically inactive forms of an enzyme and, although tools exist to determine the relation between single probes and the intron/exon structure of a target transcript in its known variants, there is no comprehensive repository providing the catalytic activity/inactivity status of different enzyme forms generated by alternative splicing.

Third, the significant number of functionally uncharacterized gene products in fully sequenced genomes, together with the errors and omissions in current biological databases can bias the results when microarray probes are used to infer affected biological functions. For example, the upper bound estimation of the fraction of enzyme-coding genes in the human genome is approximately 20%; however, the fraction of human genes currently annotated as enzymes is only 16%. Moreover, it is estimated that almost 30% of the enzyme activities that have been assigned an EC number are orphans, i.e., they have been experimentally measured in an organism but are not associated to any gene or protein sequence, either in databases or in the literature.

Fourth, the levels of mRNA estimated by microarray experiments may not closely reflect the actual protein levels. Specifically, large-scale analyses have shown a weak correlation between mRNA and protein abundance, a phenomenon that has been attributed to translational regulation, differences in protein in vivo half lives and experimental error or noise in both protein and RNA determinations.

Fifth, the qualitative treatment of metabolic flux a simplification; however, quantitative approaches such as flux balance analysis require the knowledge of the regulatory effects of covalent modifications and the kinetic constants associated to the enzymes involved in the system under study, a wealth of information that currently is both incomplete and not accurate enough to generate large-scale models.

Sixth, similarly, the very limited information available about both, subcellular location where the metabolic conversions take place and transport of metabolites between different intracellular or extracellular compartments prevents us from considering these factors in our methodology, although their influence on the in vivo levels of metabolites is evident. Information about transporter genes can be incorporated into the in silico metabolomics method, and algorithms to make use of it can be developed for qualitative metabolic flux predictions.

Finally, a factor that could confound the hypothetical correlation between lowered metabolites in cancer and their potential as therapeutic agents is the existence of moonlighting activities related to growth control exhibited by several metabolic enzymes.

By applying a fully automated method for in silico metabolomics to two different cancer cell lines nine metabolites have been discovered that alone or in combination, exhibit significant antiproliferative activity in at least one of the two cell lines. The rationale behind the findings can be described by this premise: some metabolites that have lowered levels in a cancer cell relative to normal cells contribute to the progress of the disease. The results strongly indicate that many other metabolites with important roles in carcinogenesis can be discovered or identified by the methods described herein.

In this example only cell proliferation assays have been performed, but it can be speculated that some metabolites may also exhibit other anticancer properties such as antimetastatic or antiangiogenic properties, that would not be evident as inhibition of cell growth in vitro. If the antiproliferative activities observed in cancer cell lines have a therapeutic value, different combined strategies can be devised where sets of predicted metabolites are concurrently selected according to their association with the same or different metabolic pathways, i.e., a strategy can be employed where multiple drug leads target a single pathway, or on the contrary, where each drug lead acts specifically on a different pathway.

The ligand descriptors in the third column of Table 4 include generic descriptors that refer to classes of molecules, e.g., a peptide. Many of the most general descriptors are discarded from subsequent analyses.

TABLE 4
METABOLITES PRESENT IN THE GENETIC-METABOLIC MATRIX

| | KEGG | |
|---|---|---|
| | Ligand | |
| N | identifier | KEGG Ligand description |
| 1 | C00012 | Peptide |
| 2 | C00032 | Heme; Haem; Protoheme; Heme B; Protoheme IX |
| 3 | C00039 | DNA; DNAn; DNAn + 1; (Deoxyribonucleotide)n; |
| | | (Deoxyribonucleotide)m; (Deoxyribonucleotide)n + m; Deoxyribonucleic |
| | | acid |
| 4 | C00046 | RNA; RNAn; RNAn + 1; RNA(linear); (Ribonucleotide)n; |
| | | (Ribonucleotide)m; (Ribonucleotide)n + m; Ribonucleic acid |
| 5 | C00061 | FMN; Riboflavin-5-phosphate; Flavin mononucleotide |
| 6 | C00077 | L-Ornithine; (S)-2,5-Diaminovaleric acid; (S)-2,5-Diaminopentanoic acid; |
| | | (S)-2,5-Diaminopentanoate |
| 7 | C00104 | IDP; Inosine 5′-diphosphate; Inosine diphosphate |
| 8 | C00110 | Dolichyl phosphate; Dolichol phosphate |
| 9 | C00112 | CDP; Cytidine 5′-diphosphate; Cytidine diphosphate |
| 10 | C00117 | D-Ribose 5-phosphate; Ribose 5-phosphate |
| 11 | C00119 | 5-Phospho-alpha-D-ribose 1-diphosphate; 5-Phosphoribosyl diphosphate; |
| | | 5-Phosphoribosyl 1-pyrophosphate; PRPP |
| 12 | C00120 | Biotin; D-Biotin; Vitamin H; Coenzyme R |
| 13 | C00121 | D-Ribose |
| 14 | C00129 | Isopentenyl diphosphate; delta3-Isopentenyl diphosphate; delta3-Methyl- |
| | | 3-butenyl diphosphate |
| 15 | C00130 | IMP; Inosinic acid; Inosine monophosphate; Inosine 5′-monophosphate; |
| | | Inosine 5′-phosphate; 5′-Inosinate; 5′-Inosinic acid; 5′-Inosine |
| | | monophosphate; 5′-IMP |
| 16 | C00131 | dATP; 2′-Deoxyadenosine 5′-triphosphate; Deoxyadenosine 5′- |
| | | triphosphate; Deoxyadenosine triphosphate |
| 17 | C00134 | Putrescine; 1,4-Butanediamine; 1,4-Diaminobutane; |
| | | Tetramethylenediamine |
| 18 | C00135 | L-Histidine; (S)-alpha-Amino-1H-imidazole-4-propionic acid |
| 19 | C00140 | N-Acetyl-D-glucosamine; N-Acetylchitosamine; 2-Acetamido-2-deoxy-D- |
| | | glucose; GlcNAc |
| 20 | C00143 | 5,10-Methylenetetrahydrofolate; (6R)-5,10-Methylenetetrahydrofolate; |
| | | 5,10-Methylene-THF |
| 21 | C00144 | GMP; Guanosine 5′-phosphate; Guanosine monophosphate; Guanosine 5′- |
| | | monophosphate; Guanylic acid |
| 22 | C00147 | Adenine; 6-Aminopurine |
| 23 | C00148 | L-Proline; 2-Pyrrolidinecarboxylic acid |
| 24 | C00149 | (S)-Malate; L-Malate; L-Apple acid; L-Malic acid; L-2- |
| | | Hydroxybutanedioic acid |
| 25 | C00153 | Nicotinamide; Nicotinic acid amide; Niacinamide; Vitamin PP |
| 26 | C00154 | Palmitoyl-CoA; Hexadecanoyl-CoA |
| 27 | C00157 | Phosphatidylcholine; Lecithin; Phosphatidyl-N-trimethylethanolamine; |
| | | 1,2-Diacyl-sn-glycero-3-phosphocholine; Choline phosphatide; 3-sn- |
| | | Phosphatidylcholine |
| 28 | C00158 | Citrate; Citric acid; 2-Hydroxy-1,2,3-propanetricarboxylic acid; 2- |
| | | Hydroxytricarballylic acid |
| 29 | C00160 | Glycolate; Glycolic acid; Hydroxyacetic acid |
| 30 | C00164 | Acetoacetate; 3-Oxobutanoic acid; beta-Ketobutyric acid; Acetoacetic acid |
| 31 | C00168 | Hydroxypyruvate; Hydroxypyruvic acid; 3-Hydroxypyruvate; 3- |
| | | Hydroxypyruvic acid |
| 32 | C00179 | Agmatine; (4-Aminobutyl) guanidine |
| 33 | C00183 | L-Valine; 2-Amino-3-methylbutyric acid |
| 34 | C00187 | Cholesterol; Cholest-5-en-3beta-ol |
| 35 | C00197 | 3-Phospho-D-glycerate; D-Glycerate 3-phosphate; 3-Phospho-(R)- |
| | | glycerate |
| 36 | C00206 | dADP; 2′-Deoxyadenosine 5′-diphosphate |
| 37 | C00212 | Adenosine |
| 38 | C00213 | Sarcosine; N-Methylglycine |
| 39 | C00214 | Thymidine; Deoxythymidine |
| 40 | C00219 | (5Z,8Z,11Z,14Z)-Icosatetraenoic acid; Arachidonate; Arachidonic acid; |
| | | cis-5,8,11,14-Eicosatetraenoic acid |
| 41 | C00221 | beta-D-Glucose |
| 42 | C00226 | Primary alcohol; 1-Alcohol |
| 43 | C00231 | D-Xylulose 5-phosphate |
| 44 | C00234 | 10-Formyltetrahydrofolate; 10-Formyl-THF |
| 45 | C00235 | Dimethylallyl diphosphate; Prenyl diphosphate; 2-Isopentenyl |
| | | diphosphate; delta2-Isopentenyl diphosphate; delta-Prenyl diphosphate |
| 46 | C00236 | 3-Phospho-D-glyceroyl phosphate; 1,3-Bisphospho-D-glycerate; (R)-2- |
| | | Hydroxy-3-(phosphonooxy)-1-monoanhydride with phosphoric propanoic |
| | | acid |
| 47 | C00239 | dCMP; Deoxycytidylic acid; Deoxycytidine monophosphate; |
| | | Deoxycytidylate; 2′-Deoxycytidine 5′-monophosphate |
| 48 | C00242 | Guanine; 2-Amino-6-hydroxypurine |
| 49 | C00243 | Lactose; 1-beta-D-Galactopyranosyl-4-alpha-D-glucopyranose; Milk |
| | | sugar; alpha-Lactose; Anhydrous lactose |
| 50 | C00248 | Lipoamide; Thioctic acid amide |
| 51 | C00249 | Hexadecanoic acid; Hexadecanoate; Hexadecylic acid; Palmitic acid; |
| | | Palmitate; Cetylic acid |
| 52 | C00252 | Isomaltose; Brachiose |
| 53 | C00255 | Riboflavin; Lactoflavin; 7,8-Dimethyl-10-ribitylisoalloxazine; Vitamin B2 |
| 54 | C00262 | Hypoxanthine; Purine-6-ol |
| 55 | C00268 | Dihydrobiopterin; 6,7-Dihydrobiopterin; Quinoid-dihydrobiopterin |
| 56 | C00269 | CDP-diacylglycerol; CDP-1,2-diacylglycerol; 1,2-Diacyl-sn-glycero-3- |
| | | cytidine-5′-diphosphate |
| 57 | C00272 | Tetrahydrobiopterin; 5,6,7,8-Tetrahydrobiopterin; 2-Amino-6-(1,2- |
| | | dihydroxypropyl)-5,6,7,8-tetrahydoro-4(1H)-pteridinone |
| 58 | C00275 | D-Mannose 6-phosphate |
| 59 | C00280 | Androst-4-ene-3,17-dione; Androstenedione; 4-Androstene-3,17-dione |
| 60 | C00286 | dGTP; 2′-Deoxyguanosine 5′-triphosphate; Deoxyguanosine 5′- |
| | | triphosphate; Deoxyguanosine triphosphate |
| 61 | C00288 | HCO3−; Bicarbonate; Hydrogencarbonate; Acid carbonate |
| 62 | C00293 | Glucose |
| 63 | C00294 | Inosine |
| 64 | C00295 | Orotate; Orotic acid; Uracil-6-carboxylic acid |
| 65 | C00299 | Uridine |
| 66 | C00300 | Creatine; alpha-Methylguanidino acetic acid; Methylglycocyamine |
| 67 | C00301 | ADP-ribose |
| 68 | C00307 | CDP-choline; Cytidine 5′-diphosphocholine; Citicoline |
| 69 | C00311 | Isocitrate; Isocitric acid; 1-Hydroxytricarballylic acid; 1-Hydroxypropane- |
| | | 1,2,3-tricarboxylic acid |
| 70 | C00315 | Spermidine; N-(3-Aminopropyl)-1,4-butane-diamine |
| 71 | C00319 | Sphingosine; Sphingenine; Sphingoid; Sphing-4-enine |
| 72 | C00322 | 2-Oxoadipate; 2-Oxoadipic acid |
| 73 | C00325 | GDP-L-fucose; GDP-beta-L-fucose |
| 74 | C00327 | L-Citrulline; 2-Amino-5-ureidovaleric acid; Citrulline |
| 75 | C00328 | L-Kynurenine; 3-Anthraniloyl-L-alanine |
| 76 | C00330 | Deoxyguanosine; 2′-Deoxyguanosine |
| 77 | C00332 | Acetoacetyl-CoA; Acetoacetyl coenzyme A; 3-Acetoacetyl-CoA |
| 78 | C00337 | (S)-Dihydroorotate; (S)-4,5-Dihydroorotate; L-Dihydroorotate; L- |
| | | Dihydroorotic acid; Dihydro-L-orotic acid |
| 79 | C00344 | Phosphatidylglycerol; 3-(3-sn-Phosphatidyl)glycerol; 3(3-Phosphatidyl-)glycerol; |
| | | PtdGro |
| 80 | C00345 | 6-Phospho-D-gluconate |
| 81 | C00346 | Ethanolamine phosphate; O-Phosphorylethanolamine; |
| | | Phosphoethanolamine; O-Phosphoethanolamine |
| 82 | C00350 | Phosphatidylethanolamine; (3-Phosphatidyl)ethanolamine; (3- |
| | | Phosphatidyl)-ethanolamine; Cephalin; O-(1-beta-Acyl-2-acyl-sn-glycero- |
| | | 3-phospho)ethanolamine; 1-Acyl-2-acyl-sn-glycero-3- |
| | | phosphoethanolamine |
| 83 | C00352 | D-Glucosamine 6-phosphate; D-Glucosamine phosphate |
| 84 | C00354 | D-Fructose 1,6-bisphosphate |
| 85 | C00356 | (S)-3-Hydroxy-3-methylglutaryl-CoA; Hydroxymethylglutaryl-CoA; |
| | | Hydroxymethylglutaroyl coenzyme A; HMG-CoA; 3-Hydroxy-3- |
| | | methylglutaryl-CoA |
| 86 | C00357 | N-Acetyl-D-glucosamine 6-phosphate |
| 87 | C00360 | dAMP; 2′-Deoxyadenosine 5′-phosphate; 2′-Deoxyadenosine 5′- |
| | | monophosphate; Deoxyadenylic acid; Deoxyadenosine monophosphate |
| 88 | C00361 | dGDP; 2′-Deoxyguanosine 5′-diphosphate |
| 89 | C00362 | dGMP; 2′-Deoxyguanosine 5′-monophosphate; 2′-Deoxyguanosine 5′- |
| | | phosphate; Deoxyguanylic acid; Deoxyguanosine monophosphate |
| 90 | C00364 | dTMP; Thymidine 5′-phosphate; Deoxythymidine 5′-phosphate; |
| | | Thymidylic acid; 5′-Thymidylic acid; Thymidine monophosphate; |
| | | Deoxythymidylic acid; Thymidylate |
| 91 | C00365 | dUMP; Deoxyuridylic acid; Deoxyuridine monophosphate; Deoxyuridine |
| | | 5′-phosphate; 2′-Deoxyuridine 5′-phosphate |
| 92 | C00369 | Starch |
| 93 | C00376 | Retinal; Vitamin A aldehyde; Retinene; all-trans-Retinal; all-trans-Vitamin |
| | | A aldehyde; all-trans-Retinene |
| 94 | C00379 | Xylitol |
| 95 | C00385 | Xanthine |
| 96 | C00388 | 1H-Imidazole-4-ethanamine; Histamine; 2-(4-Imidazolyl)ethylamine |
| 97 | C00390 | Ubiquinol; QH2; CoQH2 |
| 98 | C00398 | Tryptamine; 3-(2-Aminoethyl)indole |
| 99 | C00399 | Ubiquinone; Coenzyme Q; CoQ; Q |
| 100 | C00410 | Progesterone; 4-Pregnene-3,20-dione |
| 101 | C00415 | Dihydrofolate; Dihydrofolic acid; 7,8-Dihydrofolate; 7,8-Dihydrofolic |
| | | acid; 7,8-Dihydropteroylglutamate |
| 102 | C00416 | Phosphatidate; Phosphatidic acid; 1,2-Diacyl-sn-glycerol 3-phosphate; 3- |
| | | sn-Phosphatidate |
| 103 | C00417 | cis-Aconitate; cis-Aconitic acid |
| 104 | C00418 | (R)-Mevalonate; Mevalonic acid; 3,5-Dihydroxy-3-methylvaleric acid |
| 105 | C00422 | Triacylglycerol; Triglyceride |
| 106 | C00427 | Prostaglandin H2; (5Z,13E)-(15S)-9alpha,11alpha-Epidioxy-15- |
| | | hydroxyprosta-5,13-dienoate |
| 107 | C00429 | 5,6-Dihydrouracil; 2,4(1H,3H)-Pyrimidinedione, dihydro-; |
| | | Dihydrouracile; Dihydrouracil; 5,6-Dihydro-2,4-dihydroxypyrimidine; |
| | | Hydrouracil |
| 108 | C00438 | N-Carbamoyl-L-aspartate |
| 109 | C00439 | N-Formimino-L-glutamate; N-Formimidoyl-L-glutamate |
| 110 | C00440 | 5-Methyltetrahydrofolate |
| 111 | C00445 | 5,10-Methenyltetrahydrofolate |
| 112 | C00446 | alpha-D-Galactose 1-phosphate; alpha-D-Galactopyranose 1-phosphate |
| 113 | C00447 | D-Sedoheptulose 1,7-bisphosphate; D-altro-Heptulose 1,7-biphosphate |
| 114 | C00448 | trans,trans-Farnesyl diphosphate; Farnesyl diphosphate; Farnesyl |
| | | pyrophosphate; 2-trans,6-trans-Farnesyl diphosphate |
| 115 | C00449 | N6-(L-1,3-Dicarboxypropyl)-L-lysine; Saccharopine; L-Saccharopine |
| 116 | C00450 | 2,3,4,5-Tetrahydropyridine-2-carboxylate; delta1-Piperideine-6-L- |
| | | carboxylate |
| 117 | C00455 | Nicotinamide D-ribonucleotide; NMN; Nicotinamide mononucleotide; |
| | | Nicotinamide ribonucleotide; Nicotinamide nucleotide; beta-Nicotinamide |
| | | D-ribonucleotide; beta-Nicotinamide ribonucleotide; beta-Nicotinamide |
| | | mononucleotide |
| 118 | C00458 | dCTP; Deoxycytidine 5′-triphosphate; Deoxycytidine triphosphate; 2′- |
| | | Deoxycytidine 5′-triphosphate |
| 119 | C00459 | dTTP; Deoxythymidine triphosphate; Deoxythymidine 5′-triphosphate; |
| | | TTP |
| 120 | C00460 | dUTP; 2′-Deoxyuridine 5′-triphosphate |
| 121 | C00461 | Chitin; beta-1,4-Poly-N-acetyl-D-glucosamine; [1,4-(N-Acetyl-beta-D- |
| | | glucosaminyl)]n; [1,4-(N-Acetyl-beta-D-glucosaminyl)]n + 1 |
| 122 | C00468 | Estrone; 3-Hydroxy-1,3,5(10)-estratrien-17-one |
| 123 | C00469 | Ethanol; Ethyl alcohol; Methylcarbinol; Dehydrated ethanol |
| 124 | C00475 | Cytidine |
| 125 | C00483 | Tyramine; 2-(p-Hydroxyphenyl)ethylamine |
| 126 | C00486 | Bilirubin |
| 127 | C00487 | Carnitine; gamma-Trimethyl-hydroxybutyrobetaine; 3-Hydroxy-4- |
| | | trimethylammoniobutanoate |
| 128 | C00504 | Folate; Pteroylglutamic acid; Folic acid |
| 129 | C00506 | L-Cysteate; L-Cysteic acid; 3-Sulfoalanine; 2-Amino-3-sulfopropionic |
| | | acid |
| 130 | C00523 | Androsterone; 3alpha-Hydroxy-5alpha-androstan-17-one |
| 131 | C00524 | Cytochrome c |
| 132 | C00526 | Deoxyuridine; 2-Deoxyuridine; 2′-Deoxyuridine |
| 133 | C00527 | Glutaryl-CoA |
| 134 | C00532 | L-Arabitol; L-Arabinol; L-Arabinitol; L-Lyxitol |
| 135 | C00535 | Testosterone; 17beta-Hydroxy-4-androsten-3-one |
| 136 | C00546 | Methylglyoxal; Pyruvaldehyde; Pyruvic aldehyde; 2- |
| | | Ketopropionaldehyde; 2-Oxopropanal |
| 137 | C00547 | L-Noradrenaline; Noradrenaline; Norepinephrine; Arterenol; 4-[(1R)-2- |
| | | Amino-1-hydroxyethyl]-1,2-benzenediol |
| 138 | C00550 | Sphingomyelin |
| 139 | C00559 | Deoxyadenosine; 2′-Deoxyadenosine |
| 140 | C00575 | 3′,5′-Cyclic AMP; Cyclic adenylic acid; Cyclic AMP; Adenosine 3′,5′- |
| | | phosphate; cAMP |
| 141 | C00577 | D-Glyceraldehyde |
| 142 | C00579 | Dihydrolipoamide; Dihydrothioctamide |
| 143 | C00581 | Guanidinoacetate; Guanidinoacetic acid; Glycocyamine; N- |
| | | Amidinoglycine; Guanidoacetic acid |
| 144 | C00582 | Phenylacetyl-CoA |
| 145 | C00583 | Propane-1,2-diol; 1,2-Propanediol; Propylene glycol |
| 146 | C00584 | Prostaglandin E2; (5Z,13E)-(15S)-11alpha,15-Dihydroxy-9-oxoprosta- |
| | | 5,13-dienoate; (5Z,13E)-(15S)-11alpha,15-Dihydroxy-9-oxoprost-13- |
| | | enoate; Dinoprostone |
| 147 | C00588 | Choline phosphate; Phosphorylcholine; Phosphocholine; O- |
| | | Phosphocholine |
| 148 | C00606 | 3-Sulfino-L-alanine; L-Cysteinesulfinic acid; 3-Sulphino-L-alanine; 3- |
| | | Sulfinoalanine |
| 149 | C00621 | Dolichyl diphosphate; Dolichol diphosphate |
| 150 | C00624 | N-Acetyl-L-glutamate; N-Acetyl-L-glutamic acid |
| 151 | C00627 | Pyridoxine phosphate; Pyridoxine 5-phosphate; Pyridoxine 5′-phosphate |
| 152 | C00630 | 2-Methylpropanoyl-CoA; 2-Methylpropionyl-CoA; Isobutyryl-CoA |
| 153 | C00631 | 2-Phospho-D-glycerate; D-Glycerate 2-phosphate |
| 154 | C00632 | 3-Hydroxyanthranilate; 3-Hydroxyanthranilic acid |
| 155 | C00636 | D-Mannose 1-phosphate; alpha-D-Mannose 1-phosphate |
| 156 | C00643 | 5-Hydroxy-L-tryptophan |
| 157 | C00645 | N-Acetyl-D-mannosamine; 2-Acetamido-2-deoxy-D-mannose |
| 158 | C00655 | Xanthosine 5′-phosphate; Xanthylic acid; XMP; (9-D-Ribosylxanthine)-5′- |
| | | phosphate |
| 159 | C00664 | 5-Formiminotetrahydrofolate; 5-Formimidoyltetrahydrofolate |
| 160 | C00665 | beta-D-Fructose 2,6-bisphosphate; D-Fructose 2,6-bisphosphate |
| 161 | C00668 | alpha-D-Glucose 6-phosphate |
| 162 | C00669 | gamma-L-Glutamyl-L-cysteine; L-gamma-Glutamylcysteine; 5-L- |
| | | Glutamyl-L-cysteine; gamma-Glutamylcysteine |
| 163 | C00670 | sn-glycero-3-Phosphocholine; Glycerophosphocholine |
| 164 | C00673 | 2-Deoxy-D-ribose 5-phosphate |
| 165 | C00674 | 5alpha-Androstane-3,17-dione; Androstanedione |
| 166 | C00681 | 1-Acyl-sn-glycerol 3-phosphate |
| 167 | C00696 | (5Z,13E)-(15S)-9alpha,15-Dihydroxy-11-oxoprosta-5,13-dienoate; |
| | | Prostaglandin D2 |
| 168 | C00700 | XTP |
| 169 | C00705 | dCDP; 2′-Deoxycytidine diphosphate; 2′-Deoxycytidine 5′-diphosphate |
| 170 | C00718 | Amylose; Amylose chain; (1,4-alpha-D-Glucosyl)n; (1,4-alpha-D- |
| | | Glucosyl)n + 1; (1,4-alpha-D-Glucosyl)n − 1; 4-{(1,4)-alpha-D-Glucosyl}(n − |
| | | 1)-D-glucose; 1,4-alpha-D-Glucan |
| 171 | C00719 | Betaine; Trimethylaminoacetate; Glycine betaine; N,N,N- |
| | | Trimethylglycine; Trimethylammonioacetate |
| 172 | C00721 | Dextrin |
| 173 | C00735 | Cortisol; Hydrocortisone; 11beta,17alpha,21-Trihydroxy-4-pregnene-3,20- |
| | | dione; Kendall's compound F; Reichstein's substance M |
| 174 | C00750 | Spermine; N,N′-Bis(3-aminopropyl)-1,4-butanediamine |
| 175 | C00751 | Squalene; Spinacene; Supraene |
| 176 | C00762 | Cortisone; 17alpha,21-Dihydroxy-4-pregnene-3,11,20-trione; Kendall's |
| | | compound E; Reichstein's substance Fa |
| 177 | C00777 | Retinoate; Retinoic acid; Vitamin A acid; all-trans-Retinoate; Acide |
| | | retinoique (French) (DSL); Tretinoine (French) (EINECS); 3,7-Dimethyl- |
| | | 9-(2,6,6-trimethyl-1-cyclohexene-1-yl)-2,4,6,8-nonatetraenoic acid (ECL); |
| | | (all-E)-3,7-Dimethyl-9-(2,6,6-trimethyl-1-cyclohexen-1-yl)-2,4,6,8- |
| | | nonatetraenoic acid; beta-Retinoic acid; AGN 100335; all-(E)-Retinoic |
| | | acid; all-trans-beta-Retinoic acid; all-trans-Retinoic acid; all-trans- |
| | | Tretinoin; all-trans-Vitamin A acid; Ro 1-5488; trans-Retinoic acid; Tretin |
| | | M; all-trans-Vitamin A1 acid |
| 178 | C00780 | 3-(2-Aminoethyl)-1H-indol-5-ol; Serotonin; 5-Hydroxytryptamine; |
| | | Enteramine |
| 179 | C00785 | Urocanate; Urocanic acid |
| 180 | C00787 | tRNA(Tyr) |
| 181 | C00788 | L-Adrenaline; (R)-(−)-Adrenaline; (R)-(−)-Epinephrine; (R)-(−)- |
| | | Epirenamine; (R)-(−)-Adnephrine; 4-[(1R)-1-Hydroxy-2- |
| | | (methylamino)ethyl]-1,2-benzenediol |
| 182 | C00794 | D-Sorbitol; D-Glucitol; L-Gulitol; Sorbitol |
| 183 | C00818 | D-Glucarate; D-Glucaric acid; L-Gularic acid; d-Saccharic acid; D- |
| | | Glucosaccharic acid |
| 184 | C00822 | Dopaquinone |
| 185 | C00828 | Menaquinone; Menatetrenone |
| 186 | C00831 | Pantetheine; (R)-Pantetheine |
| 187 | C00836 | Sphinganine; Dihydrosphingosine; 2-Amino-1,3-dihydroxyoctadecane |
| 188 | C00842 | dTDP-glucose; dTDP-D-glucose |
| 189 | C00857 | Deamino-NAD+; Deamido-NAD+; Deamido-NAD |
| 190 | C00864 | Pantothenate; Pantothenic acid; (R)-Pantothenate |
| 191 | C00877 | Crotonoyl-CoA; Crotonyl-CoA; 2-Butenoyl-CoA; trans-But-2-enoyl-CoA; |
| | | But-2-enoyl-CoA |
| 192 | C00881 | Deoxycytidine; 2′-Deoxycytidine |
| 193 | C00882 | Dephospho-CoA |
| 194 | C00886 | L-Alanyl-tRNA; L-Alanyl-tRNA(Ala) |
| 195 | C00900 | 2-Acetolactate |
| 196 | C00906 | 5,6-Dihydrothymine; Dihydrothymine; 5,6-Dihydro-5-methyluracil |
| 197 | C00909 | Leukotriene A4; LTA4; (7E,9E,11Z,14Z)-(5S,6S)-5,6-Epoxyeicosa- |
| | | 7,9,11,14-tetraenoic acid; (7E,9E,11Z,14Z)-(5S,6S)-5,6-Epoxyeicosa- |
| | | 7,9,11,14-tetraenoate; (7E,9E,11Z,14Z)-(5S,6S)-5,6-Epoxyicosa- |
| | | 7,9,11,14-tetraenoate |
| 198 | C00931 | Porphobilinogen |
| 199 | C00942 | 3′,5′-Cyclic GMP; Guanosine 3′,5′-cyclic monophosphate; Guanosine 3′,5′- |
| | | cyclic phosphate; Cyclic GMP; cGMP |
| 200 | C00956 | L-2-Aminoadipate; L-alpha-Aminoadipate; L-alpha-Aminoadipic acid; L- |
| | | 2-Aminoadipic acid; L-2-Aminohexanedioate |
| 201 | C00957 | Mercaptopyruvate; 3-Mercaptopyruvate |
| 202 | C00962 | beta-D-Galactose |
| 203 | C00978 | N-Acetylserotonin; N-Acetyl-5-hydroxytryptamine |
| 204 | C01005 | O-Phospho-L-serine; L-O-Phosphoserine; 3-Phosphoserine |
| 205 | C01020 | 6-Hydroxynicotinate; 6-Hydroxynicotinic acid |
| 206 | C01024 | Hydroxymethylbilane |
| 207 | C01026 | N,N-Dimethylglycine; Dimethylglycine |
| 208 | C01031 | S-Formylglutathione |
| 209 | C01036 | 4-Maleylacetoacetate; 4-Maleylacetoacetic acid |
| 210 | C01042 | N-Acetyl-L-aspartate |
| 211 | C01044 | N-Formyl-L-aspartate |
| 212 | C01051 | Uroporphyrinogen III |
| 213 | C01054 | (S)-2,3-Epoxysqualene; Squalene 2,3-epoxide; Squalene 2,3-oxide; (S)- |
| | | Squalene-2,3-epoxide |
| 214 | C01059 | 2,5-Dihydroxypyridine |
| 215 | C01060 | 3,5-Diiodo-L-tyrosine; 3,5-Diiodotyrosine; L-Diiodotyrosine |
| 216 | C01061 | 4-Fumarylacetoacetate; 4-Fumarylacetoacetic acid; Fumarylacetoacetate |
| 217 | C01079 | Protoporphyrinogen IX |
| 218 | C01089 | (R)-3-Hydroxybutanoate; (R)-3-Hydroxybutanoic acid; (R)-3- |
| | | Hydroxybutyric acid |
| 219 | C01094 | D-Fructose 1-phosphate |
| 220 | C01097 | D-Tagatose 6-phosphate |
| 221 | C01102 | O-Phospho-L-homoserine |
| 222 | C01103 | Orotidine 5′-phosphate; Orotidylic acid |
| 223 | C01107 | (R)-5-Phosphomevalonate; (R)-5-Phosphomevaloonic acid; (R)-Mevalonic |
| | | acid 5-phosphate |
| 224 | C01120 | Sphinganine 1-phosphate; Dihydrosphingosine 1-phosphate |
| 225 | C01124 | 18-Hydroxycorticosterone |
| 226 | C01134 | Pantetheine 4′-phosphate; 4′-Phosphopantetheine; Phosphopantetheine; D- |
| | | Pantetheine 4′-phosphate |
| 227 | C01136 | S-Acetyldihydrolipoamide; 6-S-Acetyldihydrolipoamide |
| 228 | C01137 | S-Adenosylmethioninamine; (5-Deoxy-5-adenosyl)(3- |
| | | aminopropyl)methylsulfonium salt |
| 229 | C01143 | (R)-5-Diphosphomevalonate |
| 230 | C01144 | (S)-3-Hydroxybutanoyl-CoA; (S)-3-Hydroxybutyryl-CoA |
| 231 | C01149 | 4-Trimethylammoniobutanal |
| 232 | C01157 | trans-4-Hydroxy-L-proline |
| 233 | C01159 | 2,3-Bisphospho-D-glycerate; 2,3-Disphospho-D-glycerate; D-Greenwald |
| | | ester; DPG |
| 234 | C01161 | 3,4-Dihydroxyphenylacetate; 3,4-Dihydroxyphenylacetic acid; 3,4- |
| | | Dihydroxyphenyl acetate; 3,4-Dihydroxyphenyl acetic acid; |
| | | Homoprotocatechuate |
| 235 | C01164 | Cholesta-5,7-dien-3beta-ol; 7-Dehydrocholesterol; Provitamin D3 |
| 236 | C01165 | L-Glutamate 5-semialdehyde; L-Glutamate gamma-semialdehyde |
| 237 | C01169 | S-Succinyldihydrolipoamide |
| 238 | C01170 | UDP-N-acetyl-D-mannosamine |
| 239 | C01172 | beta-D-Glucose 6-phosphate |
| 240 | C01176 | 17alpha-Hydroxyprogesterone; 17alpha-Hydroxy-4-pregnene-3,20-dione; |
| | | Pregn-4-ene-3,20-dione-17-ol; 17alpha-Hydroxy-progesterone |
| 241 | C01177 | Inositol 1-phosphate; myo-Inositol 1-phosphate; 1D-myo-Inositol 1- |
| | | phosphate; D-myo-Inositol 1-phosphate; 1D-myo-Inositol 1- |
| | | monophosphate |
| 242 | C01181 | 4-Trimethylammoniobutanoate |
| 243 | C01185 | Nicotinate D-ribonucleotide; beta-Nicotinate D-ribonucleotide; Nicotinate |
| | | ribonucleotide; Nicotinic acid ribonucleotide |
| 244 | C01189 | 5alpha-Cholest-7-en-3beta-ol; Lathosterol |
| 245 | C01190 | Glucosylceramide; Glucocerebroside; D-Glucosyl-N-acylsphingosine |
| 246 | C01194 | 1-Phosphatidyl-D-myo-inositol; 1-Phosphatidyl-1D-myo-inositol; 1- |
| | | Phosphatidyl-myo-inositol; Phosphatidyl-1D-myo-inositol; (3- |
| | | Phosphatidyl)-1-D-inositol; 1,2-Diacyl-sn-glycero-3-phosphoinositol |
| 247 | C01204 | myo-Inositol hexakisphosphate; Phytic acid; Phytate; 1D-myo-Inositol |
| | | 1,2,3,4,5,6-hexakisphosphate; D-myo-Inositol 1,2,3,4,5,6- |
| | | hexakisphosphate; myo-Inositol 1,2,3,4,5,6-hexakisphosphate; Inositol |
| | | 1,2,3,4,5,6-hexakisphosphate; 1D-myo-Inositol hexakisphosphate |
| 248 | C01209 | Malonyl-[acyl-carrier protein] |
| 249 | C01213 | (R)-2-Methyl-3-oxopropanoyl-CoA; (R)-2-Methyl-3-oxopropionyl-CoA; |
| | | (R)-3-Oxo-2-methylpropanoyl-CoA; (R)-Methylmalonyl-CoA |
| 250 | C01220 | 1D-myo-Inositol 1,4-bisphosphate; D-myo-Inositol 1,4-bisphosphate; |
| | | myo-Inositol 1,4-bisphosphate; Inositol 1,4-bisphosphate |
| 251 | C01227 | 3beta-Hydroxyandrost-5-en-17-one; Dehydroepiandrosterone; |
| | | Dehydroisoandrosterone; DHA; DHEA |
| 252 | C01228 | Guanosine 3′,5′-bis(diphosphate); Guanosine 3′-diphosphate 5′- |
| | | diphosphate; Guanosine 5′-diphosphate,3′-diphosphate |
| 253 | C01233 | sn-glycero-3-Phosphoethanolamine; Glycerophosphoethanolamine |
| 254 | C01235 | 1-alpha-D-Galactosyl-myo-inositol; 1-O-alpha-D-Galactosyl-D-myo- |
| | | inositol; Galactinol |
| 255 | C01236 | D-Glucono-1,5-lactone 6-phosphate; 6-Phospho-D-glucono-1,5-lactone |
| 256 | C01241 | Phosphatidyl-N-methylethanolamine |
| 257 | C01242 | S-Aminomethyldihydrolipoylprotein; [Protein]-S8- |
| | | aminomethyldihydrolipoyllysine; H-Protein-S- |
| | | aminomethyldihydrolipoyllysine |
| 258 | C01243 | 1D-myo-Inositol 1,3,4-trisphosphate; D-myo-Inositol 1,3,4-trisphosphate; |
| | | Inositol 1,3,4-trisphosphate |
| 259 | C01245 | D-myo-Inositol 1,4,5-trisphosphate; 1D-myo-Inositol 1,4,5-trisphosphate; |
| | | Inositol 1,4,5-trisphosphate; Ins(1,4,5)P3 |
| 260 | C01246 | Dolichyl beta-D-glucosyl phosphate |
| 261 | C01252 | 4-(2-Aminophenyl)-2,4-dioxobutanoate |
| 262 | C01259 | 3-Hydroxy-N6,N6,N6-trimethyl-L-lysine |
| 263 | C01261 | P1,P4-Bis(5′-guanosyl) tetraphosphate; GppppG; Bis(5′-guanosyl) |
| | | tetraphosphate |
| 264 | C01272 | 1D-myo-Inositol 1,3,4,5-tetrakisphosphate; D-myo-Inositol 1,3,4,5- |
| | | tetrakisphosphate; Inositol 1,3,4,5-tetrakisphosphate |
| 265 | C01277 | 1-Phosphatidyl-1D-myo-inositol 4-phosphate; Phosphatidylinositol 4- |
| | | phosphate; 1,2-Diacyl-sn-glycero-3-phospho-(1′-myo-inositol-4′- |
| | | phosphate) |
| 266 | C01284 | 1D-myo-Inositol 1,3,4,5,6-pentakisphosphate; D-myo-Inositol 1,3,4,5,6- |
| | | pentakisphosphate; Inositol 1,3,4,5,6-pentakisphosphate |
| 267 | C01290 | beta-D-Galactosyl-1,4-beta-D-glucosylceramide; Lactosylceramide; Gal- |
| | | beta1->4Glc-beta1->1′Cer; LacCer; Lactosyl-N-acylsphingosine; D- |
| | | Galactosyl-1,4-beta-D-glucosylceramide |
| 268 | C01312 | Prostaglandin I2; (5Z,13E)-(15S)-6,9alpha-Epoxy-11alpha,15- |
| | | dihydroxyprosta-5,13-dienoate; Prostacyclin; PGI2; Epoprostenol |
| 269 | C01322 | RX |
| 270 | C01344 | dIDP; 2′-Deoxyinosine-5′-diphosphate; 2′-Deoxyinosine 5′-diphosphate |
| 271 | C01345 | dITP; 2′-Deoxyinosine-5′-triphosphate; 2′-Deoxyinosine 5′-triphosphate |
| 272 | C01346 | dUDP; 2′-Deoxyuridine 5′-diphosphate |
| 273 | C01353 | Carbonic acid; Dihydrogen carbonate; H2CO3 |
| 274 | C01412 | Butanal; Butyraldehyde |
| 275 | C01419 | Cys-Gly; L-Cysteinylglycine |
| 276 | C01528 | Selenide; Hydrogen selenide |
| 277 | C01561 | Calcidiol; 25-Hydroxyvitamin D3; Calcifediol; Calcifediol anhydrous |
| 278 | C01595 | Linoleate; Linoleic acid; (9Z,12Z)-Octadecadienoic acid; 9-cis,12-cis- |
| | | Octadecadienoate; 9-cis,12-cis-Octadecadienoic acid |
| 279 | C01596 | Maleamate; Maleamic acid |
| 280 | C01598 | Melatonin; N-Acetyl-5-methoxytryptamine |
| 281 | C01628 | Vitamin K |
| 282 | C01635 | tRNA(Ala) |
| 283 | C01636 | tRNA(Arg) |
| 284 | C01637 | tRNA(Asn) |
| 285 | C01638 | tRNA(Asp) |
| 286 | C01639 | tRNA(Cys) |
| 287 | C01640 | tRNA(Gln) |
| 288 | C01641 | tRNA(Glu) |
| 289 | C01643 | tRNA(His) |
| 290 | C01644 | tRNA(Ile) |
| 291 | C01645 | tRNA(Leu) |
| 292 | C01646 | tRNA(Lys) |
| 293 | C01647 | tRNA(Met) |
| 294 | C01648 | tRNA(Phe) |
| 295 | C01649 | tRNA(Pro) |
| 296 | C01650 | tRNA(Ser) |
| 297 | C01651 | tRNA(Thr) |
| 298 | C01652 | tRNA(Trp) |
| 299 | C01653 | tRNA(Val) |
| 300 | C01673 | Calcitriol |
| 301 | C01674 | Chitobiose |
| 302 | C01693 | L-Dopachrome; 2-L-Carboxy-2,3-dihydroindole-5,6-quinone |
| 303 | C01697 | Galactitol; Dulcitol; Dulcose |
| 304 | C01708 | Hemoglobin |
| 305 | C01724 | Lanosterol; 4,4′,14alpha-Trimethyl-5alpha-cholesta-8,24-dien-3beta-ol |
| 306 | C01753 | Sitosterol; beta-Sitosterol |
| 307 | C01762 | Xanthosine |
| 308 | C01780 | Aldosterone; 11beta,21-Dihydroxy-3,20-dioxo-4-pregnen-18-al |
| 309 | C01794 | Choloyl-CoA |
| 310 | C01798 | D-Glucoside |
| 311 | C01801 | Deoxyribose; 2-Deoxy-beta-D-erythro-pentose; Thyminose; 2-Deoxy-D- |
| | | ribose |
| 312 | C01802 | Desmosterol; 24-Dehydrocholesterol; Cholesta-5,24-dien-3beta-ol |
| 313 | C01829 | O-(4-Hydroxy-3,5-diidophenyl)-3,5-diiodo-L-tyrosine; L-Thyroxine; |
| | | 3,5,3′5′-Tetraiodo-L-thyronine; Levothyroxin |
| 314 | C01832 | Lauroyl-CoA; Lauroyl coenzyme A; Dodecanoyl-CoA |
| 315 | C01885 | 1-Acylglycerol; Glyceride; Monoglyceride; Monoacylglycerol; 1- |
| | | Monoacylglycerol |
| 316 | C01888 | Aminoacetone; 1-Amino-2-propanone |
| 317 | C01921 | Glycocholate; Glycocholic acid; 3alpha,7alpha,12alpha-Trihydroxy-5beta- |
| | | cholan-24-oylglycine |
| 318 | C01931 | L-Lysyl-tRNA; L-Lysyl-tRNA(Lys) |
| 319 | C01943 | Obtusifoliol; 4alpha,14alpha-Dimethyl-5alpha-ergosta-8,24(28)-dien- |
| | | 3beta-ol; 4alpha,14alpha-Dimethyl-24-methylene-5alpha-cholesta-8-en- |
| | | 3beta-ol |
| 320 | C01944 | Octanoyl-CoA |
| 321 | C01953 | Pregnenolone; 5-Pregnen-3beta-ol-20-one; 3beta-Hydroxypregn-5-en-20- |
| | | one |
| 322 | C01962 | Thiocysteine |
| 323 | C01996 | Acetylcholine; O-Acetylcholine |
| 324 | C02047 | L-Leucyl-tRNA; L-Leucyl-tRNA(Leu) |
| 325 | C02051 | Lipoylprotein; H-Protein-lipoyllysine |
| 326 | C02059 | Phylloquinone; Vitamin K1; Phytonadione; 2-Methyl-3-phytyl-1,4- |
| | | naphthoquinone |
| 327 | C02110 | 11-cis-Retinal; 11-cis-Vitamin A aldehyde; 11-cis-Retinene |
| 328 | C02140 | Corticosterone; 11beta,21-Dihydroxy-4-pregnene-3,20-dione; Kendall's |
| | | compound B; Reichstein's substance H |
| 329 | C02163 | L-Arginyl-tRNA(Arg); L-Arginyl-tRNA |
| 330 | C02165 | Leukotriene B4; (6Z,8E,10E,14Z)-(5S,12R)-5,12-Dihydroxyeicosa- |
| | | 6,8,10,14-tetraenoate; (6Z,8E,10E,14Z)-(5S,12R)-5,12-Dihydroxyicosa- |
| | | 6,8,10,14-tetraenoate |
| 331 | C02166 | Leukotriene C4 |
| 332 | C02188 | Protein lysine; Peptidyl-L-lysine; Procollagen L-lysine |
| 333 | C02189 | Protein serine |
| 334 | C02191 | Protoporphyrin; Protoporphyrin IX; Porphyrinogen IX |
| 335 | C02198 | Thromboxane A2; (5Z,13E)-(15S)-9alpha,11alpha-Epoxy-15- |
| | | hydroxythromboxa-5,13-dienoate; (5Z,9alpha,11alpha,13E,15S)-9,11- |
| | | Epoxy-15-hydroxythromboxa-5,13-dien-1-oic acid |
| 336 | C02218 | 2-Aminoacrylate; Dehydroalanine |
| 337 | C02282 | Glutaminyl-tRNA; L-Glutaminyl-tRNA(Gln); Glutaminyl-tRNA(Gln); |
| | | Gln-tRNA(Gln) |
| 338 | C02305 | Phosphocreatine; N-Phosphocreatine; Creatine phosphate |
| 339 | C02320 | R—S-Glutathione |
| 340 | C02336 | beta-D-Fructose; beta-Fruit sugar; beta-D-arabino-Hexulose; beta- |
| | | Levulose; Fructose |
| 341 | C02373 | 4-Methylpentanal; Isocaproaldehyde; Isohexanal |
| 342 | C02430 | L-Methionyl-tRNA; L-Methionyl-tRNA(Met) |
| 343 | C02442 | N-Methyltyramine |
| 344 | C02465 | Triiodothyronine; 3,3′5-Triiodo-L-thyronine; L-3,5,3′-Triiodothyronine; |
| | | 3,5,3′-Triiodothyronine; Liothyronine; 3,5,3′-Triiodo-L-thyronine |
| 345 | C02470 | Xanthurenic acid; Xanthurenate |
| 346 | C02492 | 1,4-beta-D-Mannan |
| 347 | C02515 | 3-Iodo-L-tyrosine |
| 348 | C02530 | Cholesterol ester |
| 349 | C02538 | Estrone 3-sulfate |
| 350 | C02553 | L-Seryl-tRNA(Ser) |
| 351 | C02554 | L-Valyl-tRNA(Val) |
| 352 | C02571 | O-Acetylcarnitine; O-Acetyl-L-carnitine |
| 353 | C02593 | Tetradecanoyl-CoA; Myristoyl-CoA |
| 354 | C02642 | 3-Ureidopropionate; 3-Ureidopropanoate; beta-Ureidopropionic acid; N- |
| | | Carbamoyl-beta-alanine |
| 355 | C02647 | 4-Guanidinobutanal |
| 356 | C02686 | Galactosylceramide; Galactocerebroside; D-Galactosyl-N- |
| | | acylsphingosine; Cerebroside; D-Galactosylceramide |
| 357 | C02700 | L-Formylkynurenine; N-Formyl-L-kynurenine; N-Formylkynurenine |
| 358 | C02702 | L-Prolyl-tRNA(Pro) |
| 359 | C02714 | N-Acetylputrescine |
| 360 | C02737 | Phosphatidylserine; Phosphatidyl-L-serine; 1,2-Diacyl-sn-glycerol 3- |
| | | phospho-L-serine; 3-O-sn-Phosphatidyl-L-serine; O3-Phosphatidyl-L- |
| | | serine |
| 361 | C02739 | Phosphoribosyl-ATP; N1-(5-Phospho-D-ribosyl)-ATP; 1-(5- |
| | | Phosphoribosyl)-ATP |
| 362 | C02763 | enol-Phenylpyruvate; enol-Phenylpyruvic acid; enol-alpha- |
| | | Ketohydrocinnamic acid; 2-Hydroxy-3-phenylpropenoate |
| 363 | C02839 | L-Tyrosyl-tRNA(Tyr) |
| 364 | C02888 | Sorbose 1-phosphate; L-Sorbose 1P; L-xylo-Hexulose 1-phosphate; L- |
| | | Sorbose 1-phosphate |
| 365 | C02918 | 1-Methylnicotinamide |
| 366 | C02934 | 3-Dehydrosphinganine; 3-Dehydro-D-sphinganine |
| 367 | C02939 | 3-Methylbutanoyl-CoA; Isovaleryl-CoA |
| 368 | C02946 | 4-Acetamidobutanoate; N4-Acetylaminobutanoate |
| 369 | C02960 | Ceramide 1-phosphate; Ceramide phosphate |
| 370 | C02972 | Dihydrolipoylprotein; [Protein]-dihydrolipoyllysine |
| 371 | C02984 | L-Aspartyl-tRNA(Asp) |
| 372 | C02985 | L-Fucose 1-phosphate; 6-Deoxy-L-galactose 1-phosphate; beta-L-Fucose |
| | | 1-phosphate |
| 373 | C02987 | L-Glutamyl-tRNA(Glu) |
| 374 | C02988 | L-Histidyl-tRNA(His) |
| 375 | C02990 | L-Palmitoylcarnitine |
| 376 | C02992 | L-Threonyl-tRNA(Thr) |
| 377 | C02999 | N-Acetylmuramoyl-Ala; N-Acetyl-D-muramoyl-L-alanine |
| 378 | C03021 | Protein asparagine; Protein L-asparagine |
| 379 | C03028 | Thiamin triphosphate; Thiamine triphosphate |
| 380 | C03033 | beta-D-Glucuronoside; Acceptor beta-D-glucuronoside; Glucuronide; |
| | | beta-D-Glucuronide |
| 381 | C03069 | 3-Methylcrotonyl-CoA; 3-Methylbut-2-enoyl-CoA; 3-Methylcrotonoyl- |
| | | CoA; Dimethylacryloyl-CoA |
| 382 | C03087 | 5-Acetamidopentanoate |
| 383 | C03090 | 5-Phosphoribosylamine; 5-Phospho-beta-D-ribosylamine; 5-Phospho-D- |
| | | ribosylamine; 5-Phosphoribosyl-1-amine |
| 384 | C03125 | L-Cysteinyl-tRNA(Cys) |
| 385 | C03127 | L-Isoleucyl-tRNA(Ile) |
| 386 | C03150 | N-Ribosylnicotinamide; 1-(beta-D-Ribofuranosyl)nicotinamide |
| 387 | C03201 | 1-Alkyl-2-acylglycerol; 2-Acyl-1-alkyl-sn-glycerol |
| 388 | C03205 | 11-Deoxycorticosterone; Deoxycorticosterone; Cortexone; 21-Hydroxy-4- |
| | | pregnene-3,20-dione; DOC |
| 389 | C03221 | 2-trans-Dodecenoyl-CoA; (2E)-Dodec-2-enoyl-CoA; (2E)-Dodecenoyl- |
| | | CoA |
| 390 | C03227 | 3-Hydroxy-L-kynurenine |
| 391 | C03231 | 3-Methylglutaconyl-CoA; trans-3-Methylglutaconyl-CoA |
| 392 | C03232 | 3-Phosphonooxypyruvate; 3-Phosphonooxypyruvic acid; 3- |
| | | Phosphohydroxypyruvate; 3-Phosphohydroxypyruvic acid |
| 393 | C03263 | Coproporphyrinogen III |
| 394 | C03267 | beta-D-Fructose 2-phosphate; beta-D-Fructofuranose 2-phosphate |
| 395 | C03284 | L-3-Amino-isobutanoate; (S)-3-Amino-isobutyrate; L-3-Amino- |
| | | isobutyrate; (S)-3-Amino-isobutanoate; (S)-3-Amino-2-methylpropanoate |
| 396 | C03287 | L-Glutamyl 5-phosphate; L-Glutamate 5-phosphate |
| 397 | C03294 | N-Formylmethionyl-tRNA |
| 398 | C03344 | 2-Methylacetoacetyl-CoA; 2-Methyl-3-acetoacetyl-CoA |
| 399 | C03345 | 2-Methylbut-2-enoyl-CoA; trans-2-Methylbut-2-enoyl-CoA; Tiglyl-CoA; |
| | | (E)-2-Methylcrotonoyl-CoA; Methylcrotonoyl-CoA; Methylcrotonyl- |
| | | CoA; Tigloyl-CoA; 2-Methylcrotanoyl-CoA |
| 400 | C03372 | Acylglycerone phosphate; Dihydroxyacetone phosphate acyl ester; 1- |
| | | Acyl-glycerone 3-phosphate |
| 401 | C03373 | Aminoimidazole ribotide; AIR; 1-(5′-Phosphoribosyl)-5-aminoimidazole; |
| | | 5′-Phosphoribosyl-5-aminoimidazole; 1-(5-Phospho-D-ribosyl)-5- |
| | | aminoimidazole; 5-Amino-1-(5-phospho-D-ribosyl)imidazole |
| 402 | C03402 | L-Asparaginyl-tRNA(Asn); Asn-tRNA(Asn); Asparaginyl-tRNA(Asn) |
| 403 | C03406 | N-(L-Arginino)succinate; N(omega)-(L-Arginino)succinate; L- |
| | | Argininosuccinate; L-Argininosuccinic acid; L-Arginosuccinic acid |
| 404 | C03410 | N-Glycoloyl-neuraminate; N-Glycolylneuraminate; NeuNGc |
| 405 | C03428 | Presqualene diphosphate |
| 406 | C03451 | (R)-S-Lactoylglutathione |
| 407 | C03460 | 2-Methylprop-2-enoyl-CoA; Methacrylyl-CoA; Methylacrylyl-CoA |
| 408 | C03479 | 5-Formyltetrahydrofolate; L(−)-5-Formyl-5,6,7,8-tetrahydrofolic acid; |
| | | Folinic acid |
| 409 | C03492 | D-4′-Phosphopantothenate; (R)-4′-Phosphopantothenate |
| 410 | C03508 | L-2-Amino-3-oxobutanoic acid; L-2-Amino-3-oxobutanoate; L-2-Amino- |
| | | acetoacetate; (S)-2-Amino-3-oxobutanoic acid |
| 411 | C03511 | L-Phenylalanyl-tRNA(Phe) |
| 412 | C03512 | L-Tryptophanyl-tRNA(Trp) |
| 413 | C03518 | N-Acetyl-D-glucosaminide |
| 414 | C03541 | Tetrahydrofolyl-[Glu](n); Tetrahydrofolyl-[Glu](n + 1); THF- |
| | | polyglutamate; Tetrahydropteroyl-[gamma-Glu]n; Tetrahydropteroyl- |
| | | [gamma-Glu]n + 1 |
| 415 | C03546 | myo-Inositol 4-phosphate; D-myo-Inositol 4-phosphate; 1D-myo-Inositol |
| | | 4-phosphate; 1D-myo-Inositol 4-monophosphate; Inositol 4-phosphate |
| 416 | C03547 | omega-Hydroxy fatty acid |
| 417 | C03594 | 7alpha-Hydroxycholesterol; Cholest-5-ene-3beta,7alpha-diol |
| 418 | C03657 | 1,4-Dihydroxy-2-naphthoate |
| 419 | C03680 | 4-Imidazolone-5-propanoate; 4-Imidazolone-5-propionic acid; 4,5- |
| | | Dihydro-4-oxo-5-imidazolepropanoate |
| 420 | C03684 | 6-Pyruvoyltetrahydropterin; 6-(1,2-Dioxopropyl)-5,6,7,8-tetrahydropterin; |
| | | 6-Pyruvoyl-5,6,7,8-tetrahydropterin |
| 421 | C03691 | CMP-N-glycoloylneuraminate; CMP-N-glycolylneuraminate; CMP- |
| | | NeuNGc |
| 422 | C03715 | O-Alkylglycerone phosphate; Alkyl-glycerone 3-phosphate; |
| | | Dihydroxyacetone phosphate alkyl ether |
| 423 | C03722 | Pyridine-2,3-dicarboxylate; Quinolinic acid; Quinolinate; 2,3- |
| | | Pyridinedicarboxylic acid |
| 424 | C03740 | (5-L-Glutamyl)-L-amino acid; L-gamma-Glutamyl-L-amino acid |
| 425 | C03758 | 4-(2-Aminoethyl)-1,2-benzenediol; 4-(2-Aminoethyl)benzene-1,2-diol; |
| | | 3,4-Dihydroxyphenethylamine; Dopamine; 2-(3,4- |
| | | Dihydroxyphenyl)ethylamine |
| 426 | C03765 | 4-Hydroxyphenylacetaldehyde; 2-(4-Hydroxyphenyl)acetaldehyde |
| 427 | C03771 | 5-Guanidino-2-oxopentanoate; 5-Guanidino-2-oxo-pentanoate; 2-Oxo-5- |
| | | guanidinopentanoate; 2-Oxo-5-guanidino-pentanoate |
| 428 | C03772 | 5beta-Androstane-3,17-dione |
| 429 | C03785 | D-Tagatose 1,6-bisphosphate |
| 430 | C03793 | N6,N6,N6-Trimethyl-L-lysine |
| 431 | C03794 | N6-(1,2-Dicarboxyethyl)-AMP; Adenylosuccinate; Adenylosuccinic acid |
| 432 | C03838 | 5′-Phosphoribosylglycinamide; GAR; N1-(5-Phospho-D- |
| | | ribosyl)glycinamide; Glycinamide ribonucleotide |
| 433 | C03845 | 5alpha-Cholest-8-en-3beta-ol; Zymostenol; Cholestenol |
| 434 | C03862 | Dolichyl phosphate D-mannose; Dolichyl D-mannosyl phosphate |
| 435 | C03892 | Phosphatidylglycerophosphate; 3(3-sn-Phosphatidyl)-sn-glycerol 1- |
| | | phosphate; 3(3-Phosphatidyl-)L-glycerol 1-phosphate; 1,2-Diacyl-sn- |
| | | glycero-3-phospho-sn-glycerol 3′-phosphate |
| 436 | C03912 | (S)-1-Pyrroline-5-carboxylate; L-1-Pyrroline-5-carboxylate; 1-Pyrroline-5- |
| | | carboxylate |
| 437 | C03917 | 17beta-Hydroxyandrostan-3-one; 5alpha-Dihydrotestosterone; |
| | | Androstanolone; 17beta-Hydroxy-5alpha-androstan-3-one |
| 438 | C03939 | Acetyl-[acyl-carrier protein] |
| 439 | C03974 | 2-Acyl-sn-glycerol 3-phosphate |
| 440 | C03981 | 2-Hydroxyethylenedicarboxylate; enol-Oxaloacetate; enol-Oxaloacetic |
| | | acid; 2-Hydroxybut-2-enedioic acid |
| 441 | C04006 | 1D-myo-Inositol 3-phosphate; D-myo-Inositol 3-phosphate; myo-Inositol |
| | | 3-phosphate; Inositol 3-phosphate; 1D-myo-Inositol 3-monophosphate; D- |
| | | myo-Inositol 3-monophosphate; myo-Inositol 3-monophosphate; Inositol |
| | | 3-monophosphate; 1L-myo-Inositol 1-phosphate; L-myo-Inositol 1- |
| | | phosphate |
| 442 | C04043 | 3,4-Dihydroxyphenylacetaldehyde; Protocatechuatealdehyde |
| 443 | C04046 | 3-D-Glucosyl-1,2-diacylglycerol; Monoglucosyldiglyceride; |
| | | Monoglucosyl-diacylglycerol; Glcbetal->3acyl2Gro |
| 444 | C04051 | 5-Amino-4-imidazolecarboxyamide |
| 445 | C04063 | D-myo-Inositol 3,4-bisphosphate; 1D-myo-Inositol 3,4-bisphosphate; |
| | | Inositol 3,4-bisphosphate |
| 446 | C04076 | L-2-Aminoadipate 6-semialdehyde; 2-Aminoadipate 6-semialdehyde |
| 447 | C04079 | N-((R)-Pantothenoyl)-L-cysteine; D-Pantothenoyl-L-cysteine; N- |
| | | Pantothenoylcysteine |
| 448 | C04185 | 5,6-Dihydroxyindole-2-carboxylate; DHICA |
| 449 | C04230 | 1-Acyl-sn-glycero-3-phosphocholine; 1-Acyl-sn-glycerol-3- |
| | | phosphocholine; alpha-Acylglycerophosphocholine; 2-Lysolecithin; 2- |
| | | Lysophosphatidylcholine; 1-Acylglycerophosphocholine |
| 450 | C04244 | 6-Lactoyl-5,6,7,8-tetrahydropterin |
| 451 | C04246 | But-2-enoyl-[acyl-carrier protein] |
| 452 | C04256 | N-Acetyl-D-glucosamine 1-phosphate |
| 453 | C04257 | N-Acetyl-D-mannosamine 6-phosphate; N-Acetylmannosamine 6- |
| | | phosphate |
| 454 | C04281 | L-1-Pyrroline-3-hydroxy-5-carboxylate; 3-Hydroxy-L-1-pyrroline-5- |
| | | carboxylate |
| 455 | C04282 | 1-Pyrroline-4-hydroxy-2-carboxylate |
| 456 | C04295 | Androst-5-ene-3beta,17beta-diol; 3beta,17beta-Dihydroxyandrost-5-ene; |
| | | 3beta,17beta-Dihydroxy-5-androstene; Androstenediol |
| 457 | C04317 | 1-Organyl-2-lyso-sn-glycero-3-phosphocholine; 1-Radyl-2-lyso-sn- |
| | | glycero-3-phosphocholine; 1-Alkyl-2-lyso-sn-glycero-3-phosphocholine; |
| | | 1-Alkyl-sn-glycero-3-phosphocholine |
| 458 | C04352 | (R)-4′-Phosphopantothenoyl-L-cysteine; N-[(R)-4′-Phosphopantothenoyl]- |
| | | L-cysteine |
| 459 | C04373 | 3alpha-Hydroxy-5beta-androstan-17-one; Etiocholan-3alpha-ol-17-one; |
| | | 3alpha-Hydroxyetiocholan-17-one |
| 460 | C04376 | 5′-Phosphoribosyl-N-formylglycinamide; N-Formyl-GAR; N- |
| | | Formylglycinamide ribonucleotide; N2-Formyl-N1-(5-phospho-D- |
| | | ribosyl)glycinamide |
| 461 | C04392 | P1,P4-Bis(5′-xanthosyl) tetraphosphate; XppppX |
| 462 | C04405 | (2S,3S)-3-Hydroxy-2-methylbutanoyl-CoA; (S)-3-Hydroxy-2- |
| | | methylbutyryl-CoA |
| 463 | C04409 | 2-Amino-3-carboxymuconate semialdehyde; 2-Amino-3-(3-oxoprop-1- |
| | | enyl)-but-2-enedioate; 2-Amino-3-(3-oxoprop-1-en-1-yl)but-2-enedioate |
| 464 | C04419 | Carboxybiotin-carboxyl-carrier protein |
| 465 | C04438 | 1-Acyl-sn-glycero-3-phosphoethanolamine; L-2- |
| | | Lysophosphatidylethanolamine |
| 466 | C04454 | 5-Amino-6-(5′-phosphoribitylamino)uracil; 5-Amino-2,6-dioxy-4-(5′- |
| | | phosphoribitylamino)pyrimidine; 5-Amino-6-(5- |
| | | phosphoribitylamino)uracil |
| 467 | C04477 | 1D-myo-Inositol 1,3,4,6-tetrakisphosphate; D-myo-Inositol 1,3,4,6- |
| | | tetrakisphosphate; Inositol 1,3,4,6-tetrakisphosphate |
| 468 | C04494 | Guanosine 3′-diphosphate 5′-triphosphate; Guanosine 5′-triphosphate,3′- |
| | | diphosphate |
| 469 | C04520 | 1D-myo-Inositol 3,4,5,6-tetrakisphosphate; D-myo-Inositol 3,4,5,6- |
| | | tetrakisphosphate; Inositol 3,4,5,6-tetrakisphosphate |
| 470 | C04546 | (R)-3-((R)-3-Hydroxybutanoyloxy)butanoate |
| 471 | C04554 | 3alpha,7alpha-Dihydroxy-5beta-cholestanate; 3alpha,7alpha-Dihydroxy- |
| | | 5beta-cholestanoate |
| 472 | C04555 | 3beta-Hydroxyandrost-5-en-17-one 3-sulfate; Dehydroepiandrosterone |
| | | sulfate |
| 473 | C04598 | 2-Acetyl-1-alkyl-sn-glycero-3-phosphocholine |
| 474 | C04618 | (3R)-3-Hydroxybutanoyl-[acyl-carrier protein]; (R)-3-Hydroxybutanoyl- |
| | | [acyl-carrier protein] |
| 475 | C04619 | (3R)-3-Hydroxydecanoyl-[acyl-carrier protein]; (R)-3-Hydroxydecanoyl- |
| | | [acyl-carrier protein] |
| 476 | C04620 | (3R)-3-Hydroxyoctanoyl-[acyl-carrier protein]; (R)-3-Hydroxyoctanoyl- |
| | | [acyl-carrier protein] |
| 477 | C04633 | (3R)-3-Hydroxypalmitoyl-[acyl-carrier protein]; (R)-3-Hydroxypalmitoyl- |
| | | [acyl-carrier protein]; (3R)-3-Hydroxyhexadecanoyl-[acyl-carrier protein]; |
| | | (R)-3-Hydroxyhexadecanoyl-[acyl-carrier protein] |
| 478 | C04637 | 1-Phosphatidyl-D-myo-inositol 4,5-bisphosphate; 1-Phosphatidyl-1D- |
| | | myo-inositol 4,5-bisphosphate; Phosphatidyl-myo-inositol 4,5- |
| | | bisphosphate; Phosphatidylinositol-4,5-bisphosphate; 1,2-Diacyl-sn- |
| | | glycero-3-phospho-(1′-myo-inositol-4′,5′-bisphosphate) |
| 479 | C04640 | 2-(Formamido)-N1-(5′-phosphoribosyl)acetamidine; 1-(5′- |
| | | Phosphoribosyl)-N-formylglycinamidine; 5′-Phosphoribosyl-N- |
| | | formylglycinamidine; 5′-Phosphoribosylformylglycinamidine; 2- |
| | | (Formamido)-N1-(5-phospho-D-ribosyl)acetamidine |
| 480 | C04644 | 3alpha,7alpha-Dihydroxy-5beta-cholestanoyl-CoA |
| 481 | C04677 | 1-(5′-Phosphoribosyl)-5-amino-4-imidazolecarboxamide; 5′- |
| | | Phosphoribosyl-5-amino-4-imidazolecarboxamide; 5′-Phospho-ribosyl-5- |
| | | amino-4-imidazole carboxamide; AICAR; 5-Aminoimidazole-4- |
| | | carboxamide ribotide; 5-Phosphoribosyl-4-carbamoyl-5-aminoimidazole; |
| | | 5-Amino-1-(5-phospho-D-ribosyl)imidazole-4-carboxamide |
| 482 | C04688 | (3R)-3-Hydroxytetradecanoyl-[acyl-carrier protein]; (R)-3- |
| | | Hydroxytetradecanoyl-[acyl-carrier protein]; beta-Hydroxymyristyl-[acyl- |
| | | carrier protein]; HMA |
| 483 | C04717 | (9Z,11E)-(13S)-13-Hydroperoxyoctadeca-9,11-dienoic acid; (9Z,11E)-(13S)- |
| | | 13-Hydroperoxyoctadeca-9,11-dienoate; 13(S)-HPODE; 13S- |
| | | Hydroperoxy-9Z,11E-octadecadienoic acid |
| 484 | C04722 | 3alpha,7alpha,12alpha-Trihydroxy-5beta-cholestanoate; |
| | | 3alpha,7alpha,12alpha-Trihydroxy-5beta-cholestan-26-oate; |
| | | 3alpha,7alpha,12alpha-Trihydroxy-5beta-cholestanate |
| 485 | C04734 | 1-(5′-Phosphoribosyl)-5-formamido-4-imidazolecarboxamide; 5′- |
| | | Phosphoribosyl-5-formamido-4-imidazolecarboxamide; 5-Formamido-1- |
| | | (5-phosphoribosyl)imidazole-4-carboxamide; 5-Formamido-1-(5-phospho- |
| | | D-ribosyl)imidazole-4-carboxamide |
| 486 | C04751 | 1-(5-Phospho-D-ribosyl)-5-amino-4-imidazolecarboxylate; 1-(5′- |
| | | Phosphoribosyl)-5-amino-4-imidazolecarboxylate; 1-(5′-Phosphoribosyl)- |
| | | 5-amino-4-carboxyimidazole; 5′-Phosphoribosyl-5-amino-4- |
| | | imidazolecarboxylate; 1-(5′-Phosphoribosyl)-4-carboxy-5- |
| | | aminoimidazole; 5′-Phosphoribosyl-4-carboxy-5-aminoimidazole; 5- |
| | | Amino-1-(5-phospho-D-ribosyl)imidazole-4-carboxylate |
| 487 | C04760 | 3alpha,7alpha,12alpha-Trihydroxy-5beta-cholestanoyl-CoA |
| 488 | C04778 | N1-(5-Phospho-alpha-D-ribosyl)-5,6-dimethylbenzimidazole; alpha- |
| | | Ribazole 5′-phosphate |
| 489 | C04805 | 5(S)-HETE; 5-Hydroxyeicosatetraenoate; 5-HETE; (6E,8Z,11Z,14Z)-(5S)- |
| | | 5-Hydroxyicosa-6,8,11,14-tetraenoic acid |
| 490 | C04823 | 1-(5′-Phosphoribosyl)-5-amino-4-(N-succinocarboxamide)-imidazole; 1- |
| | | (5′-Phosphoribosyl)-4-(N-succinocarboxamide)-5-aminoimidazole; 5′- |
| | | Phosphoribosyl-4-(N-succinocarboxamide)-5-aminoimidazole; (S)-2-[5- |
| | | Amino-1-(5-phospho-D-ribosyl)imidazole-4-carboxamido]succinate |
| 491 | C04853 | 20-OH-Leukotriene B4; 20-OH-LTB4; 20-Hydroxy-leukotriene B4; |
| | | (6Z,8E,10E,14Z)-(5S,12R)-5,12,20-Trihydroxyeicosa-6,8,10,14- |
| | | tetraenoate; (6Z,8E,10E,14Z)-(5S,12R)-5,12,20-Trihydroxyicosa- |
| | | 6,8,10,14-tetraenoate |
| 492 | C04874 | 2-Amino-4-hydroxy-6-(D-erythro-1,2,3-trihydroxypropyl)-7,8- |
| | | dihydropteridine; Dihydroneopterin |
| 493 | C04895 | 2-Amino-4-hydroxy-6-(erythro-1,2,3-trihydroxypropyl)dihydropteridine |
| | | triphosphate; 6-(L-erythro-1,2-Dihydroxypropyl 3-triphosphate)-7,8- |
| | | dihydropterin; 6-[(1S,2R)-1,2-Dihydroxy-3-triphosphooxypropyl]-7,8- |
| | | dihydropterin |
| 494 | C05100 | 3-Ureidoisobutyrate |
| 495 | C05102 | alpha-Hydroxy fatty acid |
| 496 | C05103 | 4alpha-Methylzymosterol |
| 497 | C05108 | 14-Demethyllanosterol; 4,4-Dimethyl-5alpha-cholesta-8,24-dien-3beta-ol; |
| | | 4,4-Dimethyl-8,24-cholestadienol |
| 498 | C05109 | 24,25-Dihydrolanosterol |
| 499 | C05122 | Taurocholate; Taurocholic acid; Cholyltaurine |
| 500 | C05125 | 2-(alpha-Hydroxyethyl)thiamine diphosphate; 2-Hydroxyethyl-ThPP |
| 501 | C05127 | N-Methylhistamine; 1-Methylhistamine; 1-Methyl-4-(2- |
| | | aminoethyl)imidazole |
| 502 | C05130 | Imidazole-4-acetaldehyde; Imidazole acetaldehyde |
| 503 | C05138 | 17alpha-Hydroxypregnenolone |
| 504 | C05139 | 16alpha-Hydroxydehydroepiandrosterone; 5-Androstene-3beta,16alpha- |
| | | diol-17-one |
| 505 | C05140 | 16alpha-Hydroxyandrost-4-ene-3,17-dione; 4-Androsten-16alpha-ol-3,17- |
| | | dione |
| 506 | C05141 | Estriol; 1,3,5(10)-Estratriene-3,16-alpha,17beta-triol |
| 507 | C05145 | 3-Aminoisobutanoate; 3-Amino-2-methylpropanoate |
| 508 | C05172 | Selenophosphate |
| 509 | C05200 | 3-Hexaprenyl-4,5-dihydroxybenzoate |
| 510 | C05212 | 1-Radyl-2-acyl-sn-glycero-3-phosphocholine; 1-Organyl-2-acyl-sn- |
| | | glycero-3-phosphocholine; 2-Acyl-1-alkyl-sn-glycero-3-phosphocholine |
| 511 | C05223 | Dodecanoyl-[acyl-carrier protein]; Dodecanoyl-[acp]; Lauroyl-[acyl- |
| | | carrier protein] |
| 512 | C05235 | Hydroxyacetone; Acetol; 1-Hydroxy-2-propanone; 2-Ketopropyl alcohol; |
| | | Acetone alcohol; Pyruvinalcohol; Pyruvic alcohol; Methylketol |
| 513 | C05239 | 5-Aminoimidazole; Aminoimidazole; 4-Aminoimidazole |
| 514 | C05258 | (S)-3-Hydroxyhexadecanoyl-CoA |
| 515 | C05259 | 3-Oxopalmitoyl-CoA; 3-Ketopalmitoyl-CoA; 3-Oxohexadecanoyl-CoA |
| 516 | C05260 | (S)-3-Hydroxytetradecanoyl-CoA |
| 517 | C05261 | 3-Oxotetradecanoyl-CoA |
| 518 | C05262 | (S)-3-Hydroxydodecanoyl-CoA |
| 519 | C05263 | 3-Oxododecanoyl-CoA |
| 520 | C05264 | (S)-Hydroxydecanoyl-CoA; (S)-3-Hydroxydecanoyl-CoA |
| 521 | C05265 | 3-Oxodecanoyl-CoA |
| 522 | C05266 | (S)-Hydroxyoctanoyl-CoA; (S)-3-Hydroxyoctanoyl-CoA |
| 523 | C05267 | 3-Oxooctanoyl-CoA |
| 524 | C05268 | (S)-Hydroxyhexanoyl-CoA; (S)-3-Hydroxyhexanoyl-CoA |
| 525 | C05269 | 3-Oxohexanoyl-CoA; 3-Ketohexanoyl-CoA |
| 526 | C05270 | Hexanoyl-CoA |
| 527 | C05271 | trans-Hex-2-enoyl-CoA; (2E)-Hexenoyl-CoA |
| 528 | C05272 | trans-Hexadec-2-enoyl-CoA; trans-2-Hexadecenoyl-CoA; (2E)- |
| | | Hexadecenoyl-CoA |
| 529 | C05273 | trans-Tetradec-2-enoyl-CoA; (2E)-Tetradecenoyl-CoA |
| 530 | C05274 | Decanoyl-CoA |
| 531 | C05275 | trans-Dec-2-enoyl-CoA; (2E)-Decenoyl-CoA |
| 532 | C05276 | trans-Oct-2-enoyl-CoA; (2E)-Octenoyl-CoA |
| 533 | C05279 | trans,cis-Lauro-2,6-dienoyl-CoA |
| 534 | C05280 | cis,cis-3,6-Dodecadienoyl-CoA |
| 535 | C05284 | 11beta-Hydroxyandrost-4-ene-3,17-dione; Androst-4-ene-3,17-dione- |
| | | 11beta-ol; 4-Androsten-11beta-ol-3,17-dione |
| 536 | C05285 | Adrenosterone |
| 537 | C05290 | 19-Hydroxyandrost-4-ene-3,17-dione; 19-Hydroxyandrostenedione |
| 538 | C05293 | 5beta-Dihydrotestosterone |
| 539 | C05294 | 19-Hydroxytestosterone; 17beta,19-Dihydroxyandrost-4-en-3-one |
| 540 | C05299 | 2-Methoxyestrone |
| 541 | C05300 | 16alpha-Hydroxyestrone |
| 542 | C05302 | 2-Methoxyestradiol-17beta |
| 543 | C05313 | 3-Hexaprenyl-4-hydroxy-5-methoxybenzoate |
| 544 | C05332 | Phenethylamine; 2-Phenylethylamine; beta-Phenylethylamine; |
| | | Phenylethylamine |
| 545 | C05335 | Selenomethionine |
| 546 | C05336 | Selenomethionyl-tRNA(Met) |
| 547 | C05337 | Chenodeoxycholoyl-CoA |
| 548 | C05345 | beta-D-Fructose 6-phosphate |
| 549 | C05350 | 2-Hydroxy-3-(4-hydroxyphenyl)propenoate; 4-Hydroxy-enol- |
| | | phenylpyruvate |
| 550 | C05356 | 5(S)-HPETE; 5(S)-Hydroperoxy-6-trans-8,11,14-cis-eicosatetraenoic acid; |
| | | (6E,8Z,11Z,14Z)-(5S)-5-Hydroperoxyeicosa-6,8,11,14-tetraenoate; |
| | | (6E,8Z,11Z,14Z)-(5S)-5-Hydroperoxyeicosa-6,8,11,14-tetraenoic acid |
| 551 | C05378 | beta-D-Fructose 1,6-bisphosphate |
| 552 | C05379 | Oxalosuccinate; Oxalosuccinic acid |
| 553 | C05381 | 3-Carboxy-1-hydroxypropyl-ThPP |
| 554 | C05394 | 3-Keto-beta-D-galactose |
| 555 | C05399 | Melibiitol |
| 556 | C05400 | Epimelibiose |
| 557 | C05401 | 3-beta-D-Galactosyl-sn-glycerol; Galactosylglycerol |
| 558 | C05402 | Melibiose; 6-O-(alpha-D-Galactopyranosyl)-D-glucopyranose; D-Gal- |
| | | alphal->6D-Glucose |
| 559 | C05403 | 3-Ketolactose |
| 560 | C05404 | D-Gal alpha 1->6D-Gal alpha 1->6D-Glucose; D-Gal-alpha1->6D-Gal- |
| | | alpha1->6D-Glucose; Manninotriose |
| 561 | C05406 | (4S)-5-Hydroxy-2,4-dioxopentanoate |
| 562 | C05411 | L-Xylonate |
| 563 | C05412 | L-Lyxonate |
| 564 | C05437 | Zymosterol; delta8,24-Cholestadien-3beta-ol; 5alpha-Cholesta-8,24-dien- |
| | | 3beta-ol |
| 565 | C05439 | 5alpha-Cholesta-7,24-dien-3beta-ol |
| 566 | C05444 | 3alpha,7alpha,26-Trihydroxy-5beta-cholestane; 5beta-Cholestane- |
| | | 3alpha,7alpha,26-triol |
| 567 | C05445 | 3alpha,7alpha-Dihydroxy-5beta-cholestan-26-al |
| 568 | C05447 | 3alpha,7alpha-Dihydroxy-5beta-cholest-24-enoyl-CoA |
| 569 | C05448 | 3alpha,7alpha,24-Trihydroxy-5beta-cholestanoyl-CoA |
| 570 | C05449 | 3alpha,7alpha-Dihydroxy-5beta-24-oxocholestanoyl-CoA |
| 571 | C05450 | 3alpha,7alpha,12alpha,24-Tetrahydroxy-5beta-cholestanoyl-CoA; |
| | | 3alpha,7alpha,12alpha,24zeta-Tetrahydroxy-5beta-cholestanoyl-CoA |
| 572 | C05451 | 7alpha-Hydroxy-5beta-cholestan-3-one |
| 573 | C05452 | 3alpha,7alpha-Dihydroxy-5beta-cholestane; 5beta-Cholestane- |
| | | 3alpha,7alpha-diol |
| 574 | C05453 | 7alpha,12alpha-Dihydroxy-5beta-cholestan-3-one |
| 575 | C05454 | 3alpha,7alpha,12alpha-Trihydroxy-5beta-cholestane; 5beta-Cholestane- |
| | | 3alpha,7alpha,12alpha-triol; 3alpha,7alpha,12alpha-Trihydroxycoprostane |
| 576 | C05457 | 7alpha,12alpha-Dihydroxycholest-4-en-3-one |
| 577 | C05458 | 7alpha,12alpha-Dihydroxy-5alpha-cholestan-3-one |
| 578 | C05460 | 3alpha,7alpha,12alpha-Trihydroxy-5beta-cholest-24-enoyl-CoA |
| 579 | C05461 | Chenodeoxyglycocholoyl-CoA |
| 580 | C05462 | Chenodeoxyglycocholate |
| 581 | C05467 | 3alpha,7alpha,12alpha-Trihydroxy-5beta-24-oxocholestanoyl-CoA |
| 582 | C05469 | 17alpha,21-Dihydroxy-5beta-pregnane-3,11,20-trione; 5beta-Pregnane- |
| | | 17alpha,21-diol-3,11,20-trione; 4,5beta-Dihydrocortisone |
| 583 | C05470 | Urocortisone |
| 584 | C05471 | 11beta,17alpha,21-Trihydroxy-5beta-pregnane-3,20-dione; 5beta- |
| | | Pregnane-11beta,17alpha,21-triol-3,20-dione |
| 585 | C05472 | Urocortisol; 5beta-Pregnane-3alpha,11beta,17alpha,21-tetrol-20-one |
| 586 | C05473 | 11beta,21-Dihydroxy-3,20-oxo-5beta-pregnan-18-al |
| 587 | C05474 | 3alpha,11beta,21-Trihydroxy-20-oxo-5beta-pregnan-18-al |
| 588 | C05475 | 11beta,21-Dihydroxy-5beta-pregnane-3,20-dione; 5beta-Pregnane- |
| | | 11beta,21-diol-3,20-dione |
| 589 | C05476 | Tetrahydrocorticosterone |
| 590 | C05477 | 21-Hydroxy-5beta-pregnane-3,11,20-trione |
| 591 | C05478 | 3alpha,21-Dihydroxy-5beta-pregnane-11,20-dione; 5beta-Pregnane- |
| | | 3alpha,21-diol-11,20-dione |
| 592 | C05479 | 5beta-Pregnane-3,20-dione |
| 593 | C05480 | 3alpha-Hydroxy-5beta-pregnane-20-one |
| 594 | C05485 | 21-Hydroxypregnenolone |
| 595 | C05487 | 17alpha,21-Dihydroxypregnenolone |
| 596 | C05488 | 11-Deoxycortisol; Cortodoxone (USAN) |
| 597 | C05489 | 11beta,17alpha,21-Trihydroxypregnenolone |
| 598 | C05490 | 11-Dehydrocorticosterone |
| 599 | C05497 | 21-Deoxycortisol; 4-Pregnene-11beta,17alpha-diol-3,20-dione |
| 600 | C05498 | 11beta-Hydroxyprogesterone |
| 601 | C05499 | 17alpha,20alpha-Dihydroxycholesterol |
| 602 | C05500 | 20alpha-Hydroxycholesterol |
| 603 | C05501 | 20alpha,22beta-Dihydroxycholesterol; (22R)-20alpha,22- |
| | | Dihydroxycholesterol |
| 604 | C05502 | 22beta-Hydroxycholesterol |
| 605 | C05503 | Estradiol-17beta 3-glucuronide; 17beta-Estradiol 3-(beta-D-glucuronide) |
| 606 | C05504 | 16-Glucuronide-estriol; 16alpha,17beta-Estriol 16-(beta-D-glucuronide) |
| 607 | C05512 | Deoxyinosine |
| 608 | C05516 | 5-Amino-4-imidazole carboxylate; 4-Amino-5-imidazolecarboxylic acid |
| 609 | C05527 | 3-Sulfinylpyruvate; 3-Sulfinopyruvate |
| 610 | C05528 | 3-Sulfopyruvate; 3-Sulfopyruvic acid |
| 611 | C05535 | alpha-Aminoadipoyl-S-acyl enzyme; Aminoadip.-S |
| 612 | C05543 | 3-Dehydroxycarnitine |
| 613 | C05544 | Protein N6-methyl-L-lysine |
| 614 | C05545 | Protein N6,N6-dimethyl-L-lysine |
| 615 | C05546 | Protein N6,N6,N6-trimethyl-L-lysine |
| 616 | C05548 | 6-Acetamido-2-oxohexanoate; 2-Oxo-6-acetamidocaproate |
| 617 | C05552 | N6-D-Biotinyl-L-lysine; Biocytin; epsilon-N-Biotinyl-L-lysine |
| 618 | C05560 | L-2-Aminoadipate adenylate; 5-Adenylyl-2-aminoadipate; alpha- |
| | | Aminoadipoyl-C6-AMP |
| 619 | C05576 | 3,4-Dihydroxyphenylethyleneglycol |
| 620 | C05577 | 3,4-Dihydroxymandelaldehyde |
| 621 | C05578 | 5,6-Dihydroxyindole; DHI |
| 622 | C05579 | Indole-5,6-quinone |
| 623 | C05580 | 3,4-Dihydroxymandelate |
| 624 | C05581 | 3-Methoxy-4-hydroxyphenylacetaldehyde |
| 625 | C05582 | Homovanillate; Homovanillic acid |
| 626 | C05583 | 3-Methoxy-4-hydroxyphenylglycolaldehyde |
| 627 | C05584 | 3-Methoxy-4-hydroxymandelate; Vanillylmandelic acid |
| 628 | C05585 | Gentisate aldehyde |
| 629 | C05587 | 3-Methoxytyramine |
| 630 | C05588 | L-Metanephrine |
| 631 | C05589 | L-Normetanephrine |
| 632 | C05594 | 3-Methoxy-4-hydroxyphenylethyleneglycol |
| 633 | C05596 | 4-Hydroxyphenylacetylglycine; p-Hydroxyphenylacetylglycine |
| 634 | C05598 | Phenylacetylglycine |
| 635 | C05604 | 2-Carboxy-2,3-dihydro-5,6-dihydroxyindole; Leucodopachrome |
| 636 | C05606 | Melanin |
| 637 | C05634 | 5-Hydroxyindoleacetaldehyde |
| 638 | C05635 | 5-Hydroxyindoleacetate |
| 639 | C05636 | 3-Hydroxykynurenamine |
| 640 | C05637 | 4,8-Dihydroxyquinoline; Quinoline-4,8-diol |
| 641 | C05638 | 5-Hydroxykynurenamine |
| 642 | C05639 | 4,6-Dihydroxyquinoline; Quinoline-4,6-diol |
| 643 | C05640 | Cinnavalininate |
| 644 | C05642 | Formyl-N-acetyl-5-methoxykynurenamine |
| 645 | C05643 | 6-Hydroxymelatonin |
| 646 | C05645 | 4-(2-Amino-3-hydroxyphenyl)-2,4-dioxobutanoate |
| 647 | C05647 | Formyl-5-hydroxykynurenamine |
| 648 | C05648 | 5-Hydroxy-N-formylkynurenine |
| 649 | C05651 | 5-Hydroxykynurenine |
| 650 | C05653 | Formylanthranilate; N-Formylanthranilate; 2-(Formylamino)-benzoic acid |
| 651 | C05659 | 5-Methoxytryptamine; 5-MeOT |
| 652 | C05660 | 5-Methoxyindoleacetate |
| 653 | C05665 | beta-Aminopropion aldehyde |
| 654 | C05674 | CMP-N-trimethyl-2-aminoethylphosphonate; CMP-2- |
| | | trimethylaminoethylphosphonate |
| 655 | C05676 | Diacylglyceryl-N-trimethyl-2-aminoethylphosphonate; Diacylglyceryl-2- |
| | | trimethylaminoethylphosphonate |
| 656 | C05686 | Adenylylselenate; Adenosine-5′-phosphoselenate |
| 657 | C05689 | Se-Methylselenocysteine |
| 658 | C05691 | Se-Adenosylselenomethionine |
| 659 | C05692 | Se-Adenosylselenohomocysteine |
| 660 | C05695 | gamma-Glutamyl-Se-methylselenocysteine; 5-L-Glutamyl-Se- |
| | | methylselenocysteine |
| 661 | C05696 | 3′-Phosphoadenylylselenate; 3′-Phosphoadenosine-5′-phosphoselanate |
| 662 | C05697 | Selenate; Selenic acid |
| 663 | C05698 | Selenohomocysteine |
| 664 | C05711 | gamma-Glutamyl-beta-cyanoalanine |
| 665 | C05713 | Cyanoglycoside |
| 666 | C05726 | R—S-Alanine |
| 667 | C05729 | R—S-Alanylglycine |
| 668 | C05744 | Acetoacetyl-[acp]; Acetoacetyl-[acyl-carrier protein] |
| 669 | C05745 | Butyryl-[acp]; Butyryl-[acyl-carrier protein] |
| 670 | C05746 | 3-Oxohexanoyl-[acp]; 3-Oxohexanoyl-[acyl-carrier protein] |
| 671 | C05747 | (R)-3-Hydroxyhexanoyl-[acp]; (R)-3-Hydroxyhexanoyl-[acyl-carrier |
| | | protein]; D-3-Hydroxyhexanoyl-[acp]; D-3-Hydroxyhexanoyl-[acyl- |
| | | carrier protein] |
| 672 | C05748 | trans-Hex-2-enoyl-[acp]; trans-Hex-2-enoyl-[acyl-carrier protein]; (2E)- |
| | | Hexenoyl-[acp] |
| 673 | C05749 | Hexanoyl-[acp]; Hexanoyl-[acyl-carrier protein] |
| 674 | C05750 | 3-Oxooctanoyl-[acp]; 3-Oxooctanoyl-[acyl-carrier protein] |
| 675 | C05751 | trans-Oct-2-enoyl-[acp]; trans-Oct-2-enoyl-[acyl-carrier protein]; 2- |
| | | Octenoyl-[acyl-carrier protein]; (2E)-Octenoyl-[acp] |
| 676 | C05752 | Octanoyl-[acp]; Octanoyl-[acyl-carrier protein] |
| 677 | C05753 | 3-Oxodecanoyl-[acp]; 3-Oxodecanoyl-[acyl-carrier protein] |
| 678 | C05754 | trans-Dec-2-enoyl-[acp]; trans-Dec-2-enoyl-[acyl-carrier protein]; (2E)- |
| | | Decenoyl-[acp] |
| 679 | C05755 | Decanoyl-[acp]; Decanoyl-[acyl-carrier protein] |
| 680 | C05756 | 3-Oxododecanoyl-[acp]; 3-Oxododecanoyl-[acyl-carrier protein] |
| 681 | C05757 | (R)-3-Hydroxydodecanoyl-[acp]; (R)-3-Hydroxydodecanoyl-[acyl-carrier |
| | | protein]; D-3-Hydroxydodecanoyl-[acp]; D-3-Hydroxydodecanoyl-[acyl- |
| | | carrier protein] |
| 682 | C05758 | trans-Dodec-2-enoyl-[acp]; trans-Dodec-2-enoyl-[acyl-carrier protein]; |
| | | (2E)-Dodecenoyl-[acp] |
| 683 | C05759 | 3-Oxotetradecanoyl-[acp]; 3-Oxotetradecanoyl-[acyl-carrier protein] |
| 684 | C05760 | trans-Tetradec-2-enoyl-[acp]; trans-Tetradec-2-enoyl-[acyl-carrier |
| | | protein]; (2E)-Tetradecenoyl-[acp] |
| 685 | C05761 | Tetradecanoyl-[acp]; Tetradecanoyl-[acyl-carrier protein]; Myristoyl- |
| | | [acyl-carrier protein] |
| 686 | C05762 | 3-Oxohexadecanoyl-[acp]; 3-Oxohexadecanoyl-[acyl-carrier protein] |
| 687 | C05763 | trans-Hexadec-2-enoyl-[acp]; trans-Hexadec-2-enoyl-[acyl-carrier |
| | | protein]; (2E)-Hexadecenoyl-[acp] |
| 688 | C05764 | Hexadecanoyl-[acp]; Hexadecanoyl-[acyl-carrier protein] |
| 689 | C05766 | Uroporphyrinogen I |
| 690 | C05768 | Coproporphyrinogen I |
| 691 | C05775 | alpha-Ribazole; N1-(alpha-D-ribosyl)-5,6-dimethylbenzimidazole |
| 692 | C05787 | Bilirubin beta-diglucuronide; Bilirubin-bisglucuronoside |
| 693 | C05796 | Galactan |
| 694 | C05802 | 2-Hexaprenyl-6-methoxyphenol |
| 695 | C05803 | 2-Hexaprenyl-6-methoxy-1,4-benzoquinone |
| 696 | C05804 | 2-Hexaprenyl-3-methyl-6-methoxy-1,4-benzoquinone |
| 697 | C05805 | 2-Hexaprenyl-3-methyl-5-hydroxy-6-methoxy-1,4-benzoquinone |
| 698 | C05809 | 3-Octaprenyl-4-hydroxybenzoate |
| 699 | C05810 | 2-Octaprenylphenol |
| 700 | C05813 | 2-Octaprenyl-6-methoxy-1,4-benzoquinone |
| 701 | C05814 | 2-Octaprenyl-3-methyl-6-methoxy-1,4-benzoquinone |
| 702 | C05818 | 2-Demethylmenaquinone |
| 703 | C05823 | 3-Mercaptolactate |
| 704 | C05827 | Methylimidazole acetaldehyde; 1-Methylimidazole-4-acetaldehyde; |
| | | Methylimidazoleacetaldehyde |
| 705 | C05828 | Methylimidazoleacetic acid; Tele-methylimidazoleacetic acid; 1-Methyl- |
| | | 4-imidazoleacetic acid; 1-Methylimidazole-4-acetate; |
| | | Methylimidazoleacetate |
| 706 | C05830 | 8-Methoxykynurenate |
| 707 | C05831 | 3-Methoxyanthranilate |
| 708 | C05832 | 5-Hydroxyindoleacetylglycine |
| 709 | C05841 | Nicotinate D-ribonucleoside |
| 710 | C05842 | N1-Methyl-2-pyridone-5-carboxamide; N′-Methyl-2-pyridone-5- |
| | | carboxamide |
| 711 | C05843 | N1-Methyl-4-pyridone-5-carboxamide; N′-Methyl-4-pyridone-5- |
| | | carboxamide |
| 712 | C05844 | 5-L-Glutamyl-taurine; 5-Glutamyl-taurine |
| 713 | C05849 | Vitamin K epoxide; (2,3-Epoxyphytyl)menaquinone; 1,4-Naphthoquinone, |
| | | 2,3-epoxy-2,3-dihydro-2-methyl-3-phytyl-2,3-Epoxyphylloquinone; |
| | | Naphth[2,3-b]oxirene-2,7-dione, 1a,7a-dihydro-1a-methyl-7a-(3,7,11,15- |
| | | tetramethyl-2-hexadecenyl)-Phylloquinone oxide; Phylloquinone, epoxide; |
| | | Phylloquinone-2,3-epoxide; Vitamin K 2,3-epoxide; Vitamin K1 2,3- |
| | | epoxide; Vitamin K1 oxide; Vitamin K1, epoxide; 2,3-Epoxy-2,3-dihydro- |
| | | 2-methyl-3-phytyl-1,4-naphthoquinone; 2,3-Epoxyphylloquinone |
| 714 | C05850 | Reduced Vitamin K |
| 715 | C05859 | Dehydrodolichol diphosphate; Dehydrodolichyl diphosphate |
| 716 | C05887 | N-Acetyl-D-muramoate |
| 717 | C05889 | Undecaprenyl-diphospho-N-acetylmuramoyl-(N-acetylglucosamine)-L- |
| | | alanyl-D-glutamyl-L-lysyl-D-alanyl-D-alanine |
| 718 | C05890 | Undecaprenyl-diphospho-N-acetylmuramoyl-(N-acetylglucosamine)-L- |
| | | alanyl-D-glutaminyl-L-lysyl-D-alanyl-D-alanine |
| 719 | C05894 | Undecaprenyl-diphospho-N-acetylmuramoyl-(N-acetylglucosamine)-L- |
| | | alanyl-D-isoglutaminyl-L-lysyl-D-alanyl-D-alanine |
| 720 | C05899 | Undecaprenyl-diphospho-N-acetylmuramoyl-(N-acetylglucosamine)-L- |
| | | alanyl-D-glutaminyl-meso-2,6-diaminopimeloyl-D-alanyl-D-alanine |
| 721 | C05921 | Biotinyl-5′-AMP |
| 722 | C05922 | Formamidopyrimidine nucleoside triphosphate |
| 723 | C05923 | 2,5-Diaminopyrimidine nucleoside triphosphate |
| 724 | C05925 | Dihydroneopterin phosphate; 2-Amino-4-hydroxy-6-(erythro-1,2,3- |
| | | trihydroxypropyl)dihydropteridine phosphate |
| 725 | C05933 | N-(omega)-Hydroxyarginine |
| 726 | C05935 | 2-Oxoarginine |
| 727 | C05936 | N4-Acetylaminobutanal |
| 728 | C05938 | L-4-Hydroxyglutamate semialdehyde |
| 729 | C05947 | L-erythro-4-Hydroxyglutamate |
| 730 | C05951 | Leukotriene D4; LTD4 |
| 731 | C05956 | Prostaglandin G2; PGG2 |
| 732 | C05959 | 11-epi-Prostaglandin F2alpha; 11-epi-Prostaglandin F2a; 11-epi- |
| | | PGF2alpha; 11-epi-PGF2a |
| 733 | C05966 | 15(S)-HPETE; (5Z,8Z,11Z,13E)-(15S)-15-Hydroperoxyicosa-5,8,11,13- |
| | | tetraenoic acid; 15-Hydroperoxyeicosatetraenoate; 15- |
| | | Hydroperoxyicosatetraenoate; 15-Hydroperoxyeicosatetraenoic acid; 15- |
| | | Hydroperoxyicosatetraenoic acid; (5Z,8Z,11Z,13E)-(15S)-15- |
| | | Hydroperoxyicosa-5,8,11,13-tetraenoate |
| 734 | C05977 | 2-Acyl-1-alkyl-sn-glycero-3-phosphate |
| 735 | C05980 | Cardiolipin; Diphosphatidylglycerol; 1′,3′-Bis(1,2-diacyl-sn-glycero-3- |
| | | phospho)-sn-glycerol |
| 736 | C05981 | Phosphatidylinositol-3,4,5-trisphosphate; 1-Phosphatidyl-1D-myo-inositol |
| | | 3,4,5-trisphosphate; 1,2-Diacyl-sn-glycero-3-phospho-(1′-myo-inositol- |
| | | 3′,4′,5′-bisphosphate) |
| 737 | C05983 | Propinol adenylate; Propionyladenylate |
| 738 | C05984 | 2-Hydroxybutanoic acid; 2-Hydroxybutyrate; 2-Hydroxybutyric acid |
| 739 | C05993 | Acetyl adenylate; 5′-Acetylphosphoadenosine |
| 740 | C05998 | 3-Hydroxyisovaleryl-CoA; 3-Hydroxyisovaleryl coenzyme A |
| 741 | C05999 | Lactaldehyde; 2-Hydroxypropionaldehyde; 2-Hydroxypropanal |
| 742 | C06000 | (S)-3-Hydroxyisobutyryl-CoA |
| 743 | C06001 | (S)-3-Hydroxyisobutyrate |
| 744 | C06002 | (S)-Methylmalonate semialdehyde |
| 745 | C06016 | Pentosans |
| 746 | C06017 | dTDP-D-glucuronate |
| 747 | C06023 | D-Glucosaminide |
| 748 | C06054 | 2-Oxo-3-hydroxy-4-phosphobutanoate; alpha-Keto-3-hydroxy-4- |
| | | phosphobutyrate; (3R)-3-Hydroxy-2-oxo-4-phosphonooxybutanoate |
| 749 | C06055 | O-Phospho-4-hydroxy-L-threonine; 4-(Phosphonooxy)-threonine; 4- |
| | | (Phosphonooxy)-L-threonine |
| 750 | C06056 | 4-Hydroxy-L-threonine |
| 751 | C06114 | gamma-Glutamyl-beta-aminopropiononitrile; gamma-Glutamyl-3- |
| | | aminopropiononitrile |
| 752 | C06124 | Sphingosine 1-phosphate; Sphing-4-enine 1-phosphate |
| 753 | C06125 | Sulfatide; Galactosylceramidesulfate; Cerebroside 3-sulfate |
| 754 | C06126 | Digalactosylceramide; Gal-alpha1->4Gal-beta1->1′Cer |
| 755 | C06127 | Digalactosylceramidesulfate |
| 756 | C06128 | GM4; N-Acetylneuraminyl-galactosylceramide; Neu5Ac-alpha2->3Gal- |
| | | beta1->1′Cer |
| 757 | C06142 | 1-Butanol; n-Butanol |
| 758 | C06143 | Poly-beta-hydroxybutyrate |
| 759 | C06148 | 2,5-Diamino-6-(5′-triphosphoryl-3′,4′-trihydroxy-2′-oxopentyl)-amino-4- |
| | | oxopyrimidine |
| 760 | C06157 | S-Glutaryldihydrolipoamide |
| 761 | C06196 | 2′-Deoxyinosine 5′-phosphate; dIMP |
| 762 | C06197 | P1,P3-Bis(5′-adenosyl) triphosphate; ApppA |
| 763 | C06198 | P1,P4-Bis(5′-uridyl) tetraphosphate; UppppU |
| 764 | C06199 | Hordenine; 4-[2-(Dimethylamino)ethyl]phenol |
| 765 | C06212 | N-Methylserotonin |
| 766 | C06213 | N-Methyltryptamine; N-Methylindoleethylamine; 1-Methyl-2-(3- |
| | | indolyl)ethylamine |
| 767 | C06240 | UDP-N-acetyl-D-mannosaminouronate; UDP-N-acetyl-2-amino-2-deoxy- |
| | | D-mannuronate; UDP-N-acetyl-D-mannosaminuronic acid |
| 768 | C06241 | N-Acetylneuraminate 9-phosphate |
| 769 | C06250 | Holo-[carboxylase]; Biotin-carboxyl-carrier protein |
| 770 | C06426 | (6Z,9Z,12Z)-Octadecatrienoic acid; 6,9,12-Octadecatrienoic acid; gamma- |
| | | Linolenic acid |
| 771 | C06452 | 2-Hydroxypropylphosphonate |
| 772 | C06459 | N-Trimethyl-2-aminoethylphosphonate; 2- |
| | | Trimethylaminoethylphosphonate |
| 773 | C06505 | Cob(I)yrinate a,c diamide; Cob(I)yrinate diamide; Cob(I)yrinic acid a,c- |
| | | diamide |
| 774 | C06506 | Adenosyl cobyrinate a,c diamide; Adenosyl cobyrinate diamide; |
| | | Adenosylcob(III)yrinic acid a,c-diamide; Adenosylcobyrinic acid a,c- |
| | | diamide |
| 775 | C08821 | Isofucosterol |
| 776 | C09332 | Tetrahydrofolyl-[Glu](2); THF-L-glutamate |
| 777 | C11131 | 2-Methoxy-estradiol-17beta 3-glucuronide |
| 778 | C11132 | 2-Methoxyestrone 3-glucuronide |
| 779 | C11133 | Estrone glucuronide; Estrone 3-glucuronide; Estrone beta-D-glucuronide |
| 780 | C11134 | Testosterone glucuronide; Testosterone 17beta-(beta-D-glucuronide) |
| 781 | C11135 | Androsterone glucuronide; Androsterone 3-glucuronide |
| 782 | C11136 | Etiocholan-3alpha-ol-17-one 3-glucuronide |
| 783 | C11356 | trans,trans,cis-Geranylgeranyl diphosphate; trans,trans,cis-Geranylgeranyl |
| | | pyrophosphate |
| 784 | C11455 | 4,4-Dimethyl-5alpha-cholesta-8,14,24-trien-3beta-ol |
| 785 | C11508 | 4alpha-Methyl-5alpha-ergosta-8,14,24(28)-trien-3beta-ol; delta8,14-Sterol |
| 786 | C11521 | UDP-6-sulfoquinovose |
| 787 | C11554 | 1-Phosphatidyl-1D-myo-inositol 3,4-bisphosphate; 1,2-Diacyl-sn-glycero- |
| | | 3-phospho-(1′-myo-inositol-3′,4′-bisphosphate) |
| 788 | C11555 | 1D-myo-Inositol 1,4,5,6-tetrakisphosphate; D-myo-Inositol 1,4,5,6- |
| | | tetrakisphosphate; Inositol 1,4,5,6-tetrakisphosphate |
| 789 | C12126 | Dihydroceramide; N-Acylsphinganine |
| 790 | C13309 | 2-Phytyl-1,4-naphthoquinone; Demethylphylloquinone |
| 791 | C13425 | 3-Hexaprenyl-4-hydroxybenzoate |
| 792 | C13508 | Sulfoquinovosyldiacylglycerol; SQDG; 1,2-Diacyl-3-(6-sulfo-alpha-D- |
| | | quinovosyl)-sn-glycerol |
| 793 | C13952 | UDP-N-acetyl-D-galactosaminuronic acid |
| 794 | C14748 | 20-HETE; (5Z,8Z,11Z,14Z)-20-Hydroxyicosa-5,8,11,14-tetraenoic acid; |
| | | 20-Hydroxyeicosatetraenoic acid; 20-Hydroxyicosatetraenoic acid; 20- |
| | | Hydroxy arachidonic acid |
| 795 | C14749 | 19(S)-HETE; (19S)-Hydroxyeicosatetraenoic acid; (19S)- |
| | | Hydroxyicosatetraenoic acid; (19S)-Hydroxy arachidonic acid |
| 796 | C14762 | 13(S)-HODE; (13S)-Hydroxyoctadecadienoic acid; (9Z,11E)-(13S)-13- |
| | | Hydroxyoctadeca-9,11-dienoic acid |
| 797 | C14765 | 13-OxoODE; 13-KODE; (9Z,11E)-13-Oxooctadeca-9,11-dienoic acid |
| 798 | C14768 | 5,6-EET; (8Z,11Z,14Z)-5,6-Epoxyeicosa-8,11,14-trienoic acid; |
| | | (8Z,11Z,14Z)-5,6-Epoxyicosa-8,11,14-trienoic acid |
| 799 | C14769 | 8,9-EET; (5Z,11Z,14Z)-8,9-Epoxyeicosa-5,11,14-trienoic acid; |
| | | (5Z,11Z,14Z)-8,9-Epoxyicosa-5,11,14-trienoic acid |
| 800 | C14770 | 11,12-EET; (5Z,8Z,14Z)-11,12-Epoxyeicosa-5,8,14-trienoic acid; |
| | | (5Z,8Z,14Z)-11,12-Epoxyicosa-5,8,14-trienoic acid |
| 801 | C14771 | 14,15-EET; (5Z,8Z,11Z)-14,15-Epoxyeicosa-5.8.11-trienoic acid; |
| | | (5Z,8Z,11Z)-14,15-Epoxyicosa-5.8.11-trienoic acid |
| 802 | C14772 | 5,6-DHET; (8Z,11Z,14Z)-5,6-Dihydroxyeicosa-8,11,14-trienoic acid; |
| | | (8Z,11Z,14Z)-5,6-Dihydroxyicosa-8,11,14-trienoic acid |
| 803 | C14773 | 8,9-DHET; (5Z,11Z,14Z)-8,9-Dihydroxyeicosa-5,11,14-trienoic acid; |
| | | (5Z,11Z,14Z)-8,9-Dihydroxyicosa-5,11,14-trienoic acid |
| 804 | C14774 | 11,12-DHET; (5Z,8Z,14Z)-11,12-Dihydroxyeicosa-5,8,14-trienoic acid; |
| | | (5Z,8Z,14Z)-11,12-Dihydroxyicosa-5,8,14-trienoic acid |
| 805 | C14775 | 14,15-DHET; (5Z,8Z,11Z)-14,15-Dihydroxyeicosa-5,8,11-trienoic acid; |
| | | (5Z,8Z,11Z)-14,15-Dihydroxyicosa-5,8,11-trienoic acid |
| 806 | C14778 | 16(R)-HETE; (5Z,8Z,11Z,14Z)-(16R)-16-Hydroxyeicosa-5,8,11,14- |
| | | tetraenoic acid; (5Z,8Z,11Z,14Z)-(16R)-16-Hydroxyicosa-5,8,11,14- |
| | | tetraenoic acid |
| 807 | C14781 | 15H-11,12-EETA; 15-Hydroxy-11,12-epoxyeicosatrienoic acid; |
| | | (5Z,8Z,13E)-(15S)-11,12-Epoxy-15-hydroxyeicosa-5,8,13-trienoic acid; |
| | | (5Z,8Z,13E)-(15S)-11,12-Epoxy-15-hydroxyicosa-5,8,13-trienoic acid |
| 808 | C14782 | 11,12,15-THETA; 11,12,15-Trihydroxyicosatrienoic acid; (5Z,8Z,13E)-(15S)- |
| | | 11,12,15-Trihydroxyeicosa-5,8,12-trienoic acid; (5Z,8Z,13E)-(15S)- |
| | | 11,12,15-Trihydroxyicosa-5,8,12-trienoic acid |
| 809 | C14812 | 12(R)-HPETE; (5Z,8Z,10E,14Z)-(12R)-12-Hydroperoxyeicosa-5,8,10,14- |
| | | tetraenoic acid; (5Z,8Z,10E,14Z)-(12R)-12-Hydroperoxyicosa-5,8,10,14- |
| | | tetraenoic acid |
| 810 | C14813 | 11H-14,15-EETA; 11-Hydroxy-14,15-EETA; 11-Hydroxy-14,15- |
| | | epoxyeicosatrienoic acid; (5Z,8Z,12E)-14,15-Epoxy-11-hydroxyeicosa- |
| | | 5,8,12-trienoic acid; (5Z,8Z,12E)-14,15-Epoxy-11-hydroxyicosa-5,8,12- |
| | | trienoic acid |
| 811 | C14814 | 11,14,15-THETA; 11,14,15-Trihydroxyicosatrienoic acid; (5Z,8Z,12E)- |
| | | 11,14,15-Trihydroxyeicosa-5,8,12-trienoic acid; (5Z,8Z,12E)-11,14,15- |
| | | Trihydroxyicosa-5,8,12-trienoic acid |
| 812 | C14818 | Fe2+; Fe(II); Ferrous ion; Iron(2+) |
| 813 | C14819 | Fe3+; Fe(III); Ferric ion; Iron(3+) |
| 814 | C14823 | 8(S)-HPETE; (5Z,9E,11Z,14Z)-(8S)-8-Hydroperoxyeicosa-5,9,11,14- |
| | | tetraenoic acid; (5Z,9E,11Z,14Z)-(8S)-8-Hydroperoxyicosa-5,9,11,14- |
| | | tetraenoic acid |
| 815 | C14825 | 9(10)-EpOME; (9R,10S)-(12Z)-9,10-Epoxyoctadecenoic acid |
| 816 | C14826 | 12(13)-EpOME; (12R,13S)-(9Z)-12,13-Epoxyoctadecenoic acid |
| 817 | C14827 | 9(S)-HPODE; 9(S)-HPOD; (10E,12Z)-(9S)-9-Hydroperoxyoctadeca- |
| | | 10,12-dienoic acid |
| 818 | C15645 | 1-(1-Alkenyl)-sn-glycerol |
| 819 | C15647 | 2-Acyl-1-(1-alkenyl)-sn-glycero-3-phosphate |
| 820 | C15670 | Heme A |
| 821 | C15672 | Heme O |
| 822 | C15776 | 4alpha-Methylfecosterol |
| 823 | C15780 | 5-Dehydroepisterol |
| 824 | C15781 | 24-Methylenecholesterol |
| 825 | C15782 | delta7-Avenasterol |
| 826 | C15783 | 5-Dehydroavenasterol |
| 827 | C15808 | 4alpha-Methylzymosterol-4-carboxylate; 4alpha-Carboxy-4beta-methyl- |
| | | 5alpha-cholesta-8,24-dien-3beta-ol |
| 828 | C15811 | C15811; Thiamine biosynthesis intermediate 2 |
| 829 | C15812 | C15812; Thiamine biosynthesis intermediate 3 |
| 830 | C15816 | 3-Keto-4-methylzymosterol |
| 831 | C15915 | 4,4-Dimethyl-5alpha-cholesta-8-en-3beta-ol |
| 832 | C15972 | Enzyme N6-(lipoyl)lysine; Lipoamide-E |
| 833 | C15973 | Enzyme N6-(dihydrolipoyl)lysine; Dihydrolipoamide-E |
| 834 | C15974 | 3-Methyl-1-hydroxybutyl-ThPP; 3-Methyl-1-hydroxybutyl-TPP |
| 835 | C15975 | [Dihydrolipoyllysine-residue (2-methylpropanoyl)transferase] S-(3- |
| | | methylbutanoyl)dihydrolipoyllysine; S-(3-Methylbutanoyl)- |
| | | dihydrolipoamide-E |
| 836 | C15976 | 2-Methyl-1-hydroxypropyl-ThPP; 2-Methyl-1-hydroxypropyl-TPP |
| 837 | C15977 | [Dihydrolipoyllysine-residue (2-methylpropanoyl)transferase] S-(2- |
| | | methylpropanoyl)dihydrolipoyllysine; S-(2-Methylpropanoyl)- |
| | | dihydrolipoamide-E; S-(2-Methylpropionyl)-dihydrolipoamide-E |
| 838 | C15978 | 2-Methyl-1-hydroxybutyl-ThPP; 2-Methyl-1-hydroxybutyl-TPP |
| 839 | C15979 | [Dihydrolipoyllysine-residue (2-methylpropanoyl)transferase] S-(2- |
| | | methylbutanoyl)dihydrolipoyllysine; S-(2-Methylbutanoyl)- |
| | | dihydrolipoamide-E |
| 840 | C15980 | (S)-2-Methylbutanoyl-CoA |
| 841 | G00001 | N-Acetyl-D-glucosaminyldiphosphodolichol; (GlcNAc)1 (PP-Dol)1 |
| 842 | G00002 | N,N′-Chitobiosyldiphosphodolichol; (GlcNAc)2 (PP-Dol)1 |
| 843 | G00003 | (GlcNAc)2 (Man)1 (PP-Dol)1 |
| 844 | G00004 | (GlcNAc)2 (Man)2 (PP-Dol)1 |
| 845 | G00005 | (GlcNAc)2 (Man)3 (PP-Dol)1 |
| 846 | G00006 | (GlcNAc)2 (Man)5 (PP-Dol)1 |
| 847 | G00007 | (GlcNAc)2 (Man)9 (PP-Dol)1 |
| 848 | G00008 | (Glc)3 (GlcNAc)2 (Man)9 (PP-Dol)1 |
| 849 | G00009 | (Glc)3 (GlcNAc)2 (Man)9 (Asn)1; Glycoprotein; N-Glycan |
| 850 | G00010 | (Glc)1 (GlcNAc)2 (Man)9 (Asn)1; Glycoprotein; N-Glycan |
| 851 | G00011 | (GlcNAc)2 (Man)9 (Asn)1; Glycoprotein; N-Glycan |
| 852 | G00012 | (GlcNAc)2 (Man)5 (Asn)1; Glycoprotein; N-Glycan |
| 853 | G00013 | (GlcNAc)3 (Man)5 (Asn)1; Glycoprotein; N-Glycan |
| 854 | G00014 | (GlcNAc)3 (Man)3 (Asn)1; Glycoprotein; N-Glycan |
| 855 | G00015 | (GlcNAc)4 (Man)3 (Asn)1; Glycoprotein; N-Glycan |
| 856 | G00016 | (GlcNAc)4 (LFuc)1 (Man)3 (Asn)1; Glycoprotein; N-Glycan |
| 857 | G00017 | (Gal)2 (GlcNAc)4 (LFuc)1 (Man)3 (Asn)1; Glycoprotein; N-Glycan |
| 858 | G00018 | DS 3; (Gal)2 (GlcNAc)4 (LFuc)1 (Man)3 (Neu5Ac)2 (Asn)1; |
| | | Glycoprotein; N-Glycan |
| 859 | G00019 | (GlcNAc)5 (Man)3 (Asn)1; Glycoprotein; N-Glycan |
| 860 | G00020 | (GlcNAc)5 (Man)3 (Asn)1; Glycoprotein; N-Glycan |
| 861 | G00021 | (GlcNAc)6 (Man)3 (Asn)1; Glycoprotein; N-Glycan |
| 862 | G00023 | Tn antigen; (GalNAc)1 (Ser/Thr)1; Glycoprotein; O-Glycan |
| 863 | G00024 | T antigen; (Gal)1 (GalNAc)1 (Ser/Thr)1; Glycoprotein; O-Glycan |
| | | Neoglycoconjugate |
| 864 | G00025 | (Gal)1 (GalNAc)1 (GlcNAc)1 (Ser/Thr)1; Glycoprotein; O-Glycan |
| 865 | G00026 | (Gal)1 (GalNAc)1 (Neu5Ac)1 (Ser/Thr)1; Glycoprotein; O-Glycan |
| 866 | G00027 | (Gal)1 (GalNAc)1 (Neu5Ac)2 (Ser/Thr)1; Glycoprotein; O-Glycan |
| 867 | G00028 | (GalNAc)1 (GlcNAc)1 (Ser/Thr)1; Glycoprotein; O-Glycan |
| 868 | G00029 | (GalNAc)1 (GlcNAc)2 (Ser/Thr)1; Glycoprotein; O-Glycan |
| 869 | G00031 | (GalNAc)1 (GlcNAc)1 (Ser/Thr)1; Glycoprotein; O-Glycan |
| 870 | G00032 | (Gal)1 (GalNAc)1 (GlcNAc)1 (Ser/Thr)1; Glycoprotein; O-Glycan |
| 871 | G00035 | Sialyl-Tn antigen; (GalNAc)1 (Neu5Ac)1 (Ser/Thr)1; Glycoprotein; O- |
| | | Glycan |
| 872 | G00036 | Lc3Cer; (Gal)1 (Glc)1 (GlcNAc)1 (Cer)1; Glycolipid; Sphingolipid |
| 873 | G00037 | Lc4Cer; (Gal)2 (Glc)1 (GlcNAc)1 (Cer)1; Glycolipid; Sphingolipid |
| 874 | G00038 | (Gal)3 (Glc)1 (GlcNAc)1 (Cer)1; Glycolipid; Sphingolipid |
| 875 | G00039 | Type IB glycolipid; (Gal)3 (Glc)1 (GlcNAc)1 (LFuc)1 (Cer)1; Glycolipid; |
| | | Sphingolipid |
| 876 | G00040 | (Gal)3 (Glc)1 (GlcNAc)1 (LFuc)2 (Cer)1; Glycolipid; Sphingolipid |
| 877 | G00042 | Type IA glycolipid; (Gal)2 (GalNAc)1 (Glc)1 (GlcNAc)1 (LFuc)1 (Cer)1; |
| | | Glycolipid; Sphingolipid |
| 878 | G00043 | (Gal)2 (GalNAc)1 (Glc)1 (GlcNAc)1 (LFuc)2 (Cer)1; Glycolipid; |
| | | Sphingolipid |
| 879 | G00044 | IV2Fuc-Lc4Cer; IV2-a-Fuc-Lc4Cer; Type IH glycolipid; (Gal)2 (Glc)1 |
| | | (GlcNAc)1 (LFuc)1 (Cer)1; Glycolipid; Sphingolipid |
| 880 | G00045 | IV2Fuc,III4Fuc-Lc4Cer; IV2-a-Fuc,III4-a-Fuc-Lc4Cer; Leb glycolipid; |
| | | (Gal)2 (Glc)1 (GlcNAc)1 (LFuc)2 (Cer)1; Glycolipid; Sphingolipid |
| 881 | G00046 | Fuc-Lc4Cer; III4-a-Fuc-Lc4Cer; Lea glycolipid; (Gal)2 (Glc)1 (GlcNAc)1 |
| | | (LFuc)1 (Cer)1; Glycolipid; Sphingolipid |
| 882 | G00047 | 3′-isoLM1; IV3-a-Neu5Ac-Lc4Cer; sLc4Cer; (Gal)2 (Glc)1 (GlcNAc)1 |
| | | (Neu5Ac)1 (Cer)1; Glycolipid; Sphingolipid |
| 883 | G00048 | Fuc-3′-isoLM1; IV3-a-Neu5Ac,III4-a-Fuc-Lc4Cer; (Gal)2 (Glc)1 |
| | | (GlcNAc)1 (LFuc)1 (Neu5Ac)1 (Cer)1; Glycolipid; Sphingolipid |
| 884 | G00050 | Paragloboside; Lactoneotetraosylceramide; Lacto-N-neotetraosylceramide; |
| | | Neolactotetraosylceramide; LA1; nLcCer; (Gal)2 (Glc)1 (GlcNAc)1 |
| | | (Cer)1; Glycolipid; Sphingolipid |
| 885 | G00051 | nLc5Cer; (Gal)3 (Glc)1 (GlcNAc)1 (Cer)1; Glycolipid; Sphingolipid |
| 886 | G00052 | Type II B antigen; (Gal)3 (Glc)1 (GlcNAc)1 (LFuc)1 (Cer)1; Glycolipid; |
| | | Sphingolipid |
| 887 | G00054 | Type II A antigen; (Gal)2 (GalNAc)1 (Glc)1 (GlcNAc)1 (LFuc)1 (Cer)1; |
| | | Glycolipid; Sphingolipid |
| 888 | G00055 | IV2Fuc-nLc4Cer; IV2-a-Fuc-nLc4Cer; Type IIH glycolipid; (Gal)2 (Glc)1 |
| | | (GlcNAc)1 (LFuc)1 (Cer)1; Glycolipid; Sphingolipid |
| 889 | G00056 | III3,IV2Fuc-nLc4Cer; IV2-a-Fuc,III3-a-Fuc-nLc4Cer; Ley glycolipid; |
| | | (Gal)2 (Glc)1 (GlcNAc)1 (LFuc)2 (Cer)1; Glycolipid; Sphingolipid |
| 890 | G00057 | (Gal)3 (GalNAc)1 (Glc)1 (GlcNAc)1 (LFuc)1 (Cer)1; Glycolipid; |
| | | Sphingolipid |
| 891 | G00058 | Type IIIH glycolipid; (Gal)3 (GalNAc)1 (Glc)1 (GlcNAc)1 (LFuc)2 |
| | | (Cer)1; Glycolipid; Sphingolipid |
| 892 | G00059 | Type IIIA glycolipid; (Gal)3 (GalNAc)2 (Glc)1 (GlcNAc)1 (LFuc)2 |
| | | (Cer)1; Glycolipid; Sphingolipid |
| 893 | G00060 | III3Fuc-nLc4Cer; III3-a-Fuc-nLc4Cer; Lacto-N-fucopentaosyl III |
| | | ceramide; LNF III cer; SSEA-1; (Gal)2 (Glc)1 (GlcNAc)1 (LFuc)1 (Cer)1; |
| | | Glycolipid; Sphingolipid |
| 894 | G00062 | Sialyl-3-paragloboside; 3′-LM1; IV3-a-Neu5Ac-nLc4Cer; snLc4Cer; |
| | | (Gal)2 (Glc)1 (GlcNAc)1 (Neu5Ac)1 (Cer)1; Glycolipid; Sphingolipid |
| 895 | G00063 | IV3NeuAc,III3Fuc-nLc4Cer; IV3-a-NeuAc,III3-a-Fuc-nLc4Cer; (Gal)2 |
| | | (Glc)1 (GlcNAc)1 (LFuc)1 (Neu5Ac)1 (Cer)1; Glycolipid; Sphingolipid |
| 896 | G00064 | 3′,8′-LD1; (Gal)2 (Glc)1 (GlcNAc)1 (Neu5Ac)2 (Cer)1; Glycolipid; |
| | | Sphingolipid |
| 897 | G00066 | nLc5Cer; (Gal)2 (Glc)1 (GlcNAc)2 (Cer)1; Glycolipid; Sphingolipid |
| 898 | G00067 | nLc6Cer; i-antigen; (Gal)3 (Glc)1 (GlcNAc)2 (Cer)1; Glycolipid; |
| | | Sphingolipid |
| 899 | G00068 | nLc7Cer; (Gal)3 (Glc)1 (GlcNAc)3 (Cer)1; Glycolipid; Sphingolipid |
| 900 | G00069 | nLc8Cer; (Gal)4 (Glc)1 (GlcNAc)3 (Cer)1; Glycolipid; Sphingolipid |
| 901 | G00071 | VI2Fuc-nLc6; (Gal)3 (Glc)1 (GlcNAc)2 (LFuc)1 (Cer)1; Glycolipid; |
| | | Sphingolipid |
| 902 | G00072 | (Gal)3 (GalNAc)1 (Glc)1 (GlcNAc)2 (LFuc)1 (Cer)1; Glycolipid; |
| | | Sphingolipid |
| 903 | G00073 | (Gal)4 (GalNAc)1 (Glc)1 (GlcNAc)2 (LFuc)1 (Cer)1; Glycolipid; |
| | | Sphingolipid |
| 904 | G00074 | (Gal)4 (GalNAc)1 (Glc)1 (GlcNAc)2 (LFuc)2 (Cer)1; Glycolipid; |
| | | Sphingolipid |
| 905 | G00075 | Type IIIAb; (Gal)4 (GalNAc)2 (Glc)1 (GlcNAc)2 (LFuc)2 (Cer)1; |
| | | Glycolipid; Sphingolipid |
| 906 | G00076 | III3Fuc-nLc6Cer; (Gal)3 (Glc)1 (GlcNAc)2 (LFuc)1 (Cer)1; Glycolipid; |
| | | Sphingolipid |
| 907 | G00077 | (Gal)3 (Glc)1 (GlcNAc)3 (Cer)1; Glycolipid; Sphingolipid |
| 908 | G00078 | iso-nLc8Cer; LacNAc-Lc6Cer; I-antigen; Lactoisooctaosylceramide; |
| | | (Gal)4 (Glc)1 (GlcNAc)3 (Cer)1; Glycolipid; Sphingolipid |
| 909 | G00079 | (Gal)4 (Glc)1 (GlcNAc)3 (LFuc)2 (Cer)1; Glycolipid; Sphingolipid |
| 910 | G00081 | (Gal)3 (Glc)1 (GlcNAc)2 (LFuc)2 (Cer)1; Glycolipid; Sphingolipid |
| 911 | G00082 | (Gal)3 (Glc)1 (GlcNAc)2 (LFuc)3 (Cer)1; Glycolipid; Sphingolipid |
| 912 | G00083 | (Gal)4 (Glc)1 (GlcNAc)2 (LFuc)1 (Cer)1; Glycolipid; Sphingolipid |
| 913 | G00084 | (Gal)4 (Glc)1 (GlcNAc)3 (LFuc)1 (Cer)1; Glycolipid; Sphingolipid |
| 914 | G00085 | (Gal)4 (Glc)1 (GlcNAc)3 (LFuc)2 (Cer)1; Glycolipid; Sphingolipid |
| 915 | G00086 | (Gal)4 (Glc)1 (GlcNAc)3 (LFuc)3 (Cer)1; Glycolipid; Sphingolipid |
| 916 | G00088 | VI3NeuAc-nLc6Cer; (Gal)3 (Glc)1 (GlcNAc)2 (Neu5Ac)1 (Cer)1; |
| | | Glycolipid; Sphingolipid |
| 917 | G00089 | V3Fuc-nLc6Cer; (Gal)3 (Glc)1 (GlcNAc)2 (LFuc)1 (Cer)1; Glycolipid; |
| | | Sphingolipid |
| 918 | G00090 | V3Fuc,III3Fuc-nLc6Cer; (Gal)3 (Glc)1 (GlcNAc)2 (LFuc)2 (Cer)1; |
| | | Glycolipid; Sphingolipid |
| 919 | G00092 | Lactosylceramide; CDw17; LacCer; (Gal)1 (Glc)1 (Cer)1; Glycolipid; |
| | | Sphingolipid |
| 920 | G00093 | Globotriaosylceramide; Gb3Cer; Pk antigen; CD77; (Gal)2 (Glc)1 (Cer)1; |
| | | Glycolipid; Sphingolipid |
| 921 | G00094 | Globoside; Gb4Cer; P antigen; (Gal)2 (GalNAc)1 (Glc)1 (Cer)1; |
| | | Glycolipid; Sphingolipid |
| 922 | G00095 | IV3GalNAca-Gb4Cer; (Gal)2 (GalNAc)2 (Glc)1 (Cer)1; Glycolipid; |
| | | Sphingolipid |
| 923 | G00097 | Galactosylgloboside; SSEA-3; Gb5Cer; (Gal)3 (GalNAc)1 (Glc)1 (Cer)1; |
| | | Glycolipid; Sphingolipid |
| 924 | G00098 | Monosialylgalactosylgloboside; MSGG; Monosialyl-Gb5; SSEA-4; |
| | | V3NeuAc-Gb5Cer; (Gal)3 (GalNAc)1 (Glc)1 (Neu5Ac)1 (Cer)1; |
| | | Glycolipid; Sphingolipid |
| 925 | G00099 | Globo-H; (Gal)3 (GalNAc)1 (Glc)1 (LFuc)1 (Cer)1; Glycolipid; |
| | | Sphingolipid |
| 926 | G00102 | (Gal)3 (GalNAc)1 (Glc)1 (GlcNAc)1 (Cer)1; Glycolipid; Sphingolipid |
| 927 | G00103 | (Gal)4 (GalNAc)1 (Glc)1 (GlcNAc)1 (Cer)1; Glycolipid; Sphingolipid |
| 928 | G00104 | (Gal)4 (GalNAc)1 (Glc)1 (GlcNAc)1 (LFuc)1 (Cer)1; Glycolipid; |
| | | Sphingolipid |
| 929 | G00108 | GM3; Hematoside; (Gal)1 (Glc)1 (Neu5Ac)1 (Cer)1; Glycolipid; |
| | | Sphingolipid |
| 930 | G00109 | GM2; Ganglioside; (Gal)1 (GalNAc)1 (Glc)1 (Neu5Ac)1 (Cer)1; |
| | | Glycolipid; Sphingolipid |
| 931 | G00110 | GM1; (Gal)2 (GalNAc)1 (Glc)1 (Neu5Ac)1 (Cer)1; Glycolipid; |
| | | Sphingolipid |
| 932 | G00111 | GD1a; (Gal)2 (GalNAc)1 (Glc)1 (Neu5Ac)2 (Cer)1; Glycolipid; |
| | | Sphingolipid |
| 933 | G00112 | GT1a; (Gal)2 (GalNAc)1 (Glc)1 (Neu5Ac)3 (Cer)1; Glycolipid; |
| | | Sphingolipid |
| 934 | G00113 | GD3; CD60a; (Gal)1 (Glc)1 (Neu5Ac)2 (Cer)1; Glycolipid; Sphingolipid |
| 935 | G00114 | GD2; (Gal)1 (GalNAc)1 (Glc)1 (Neu5Ac)2 (Cer)1; Glycolipid; |
| | | Sphingolipid |
| 936 | G00115 | GD1b; (Gal)2 (GalNAc)1 (Glc)1 (Neu5Ac)2 (Cer)1; Glycolipid; |
| | | Sphingolipid |
| 937 | G00116 | GT1b; (Gal)2 (GalNAc)1 (Glc)1 (Neu5Ac)3 (Cer)1; Glycolipid; |
| | | Sphingolipid |
| 938 | G00117 | GQ1b; (Gal)2 (GalNAc)1 (Glc)1 (Neu5Ac)4 (Cer)1; Glycolipid; |
| | | Sphingolipid |
| 939 | G00118 | GT3; (Gal)1 (Glc)1 (Neu5Ac)3 (Cer)1; Glycolipid; Sphingolipid |
| 940 | G00119 | GT2; (Gal)1 (GalNAc)1 (Glc)1 (Neu5Ac)3 (Cer)1; Glycolipid; |
| | | Sphingolipid |
| 941 | G00120 | GT1c; (Gal)2 (GalNAc)1 (Glc)1 (Neu5Ac)3 (Cer)1; Glycolipid; |
| | | Sphingolipid |
| 942 | G00123 | GA2; (Gal)1 (GalNAc)1 (Glc)1 (Cer)1; Glycolipid; Sphingolipid |
| 943 | G00124 | GA1; (Gal)2 (GalNAc)1 (Glc)1 (Cer)1; Glycolipid; Sphingolipid |
| 944 | G00125 | GM1b; (Gal)2 (GalNAc)1 (Glc)1 (Neu5Ac)1 (Cer)1; Glycolipid; |
| | | Sphingolipid |
| 945 | G00126 | GD1c; (Gal)2 (GalNAc)1 (Glc)1 (Neu5Ac)2 (Cer)1; Glycolipid; |
| | | Sphingolipid |
| 946 | G00127 | GD1a; (Gal)2 (GalNAc)1 (Glc)1 (Neu5Ac)2 (Cer)1; Glycolipid; |
| | | Sphingolipid |
| 947 | G00128 | GT1aalpha; (Gal)2 (GalNAc)1 (Glc)1 (Neu5Ac)3 (Cer)1; Glycolipid; |
| | | Sphingolipid |
| 948 | G00129 | GQ1balpha; (Gal)2 (GalNAc)1 (Glc)1 (Neu5Ac)4 (Cer)1; Glycolipid; |
| | | Sphingolipid |
| 949 | G00140 | (GlcN)1 (Ino(acyl)-P)1 (Man)4 (EtN)1 (P)1; Glycoprotein; GPI anchor |
| 950 | G00141 | (GlcN)1 (Ino(acyl)-P)1 (Man)4 (EtN)2 (P)2; Glycoprotein; GPI anchor |
| 951 | G00143 | (GlcNAc)1 (Ino-P)1; Glycoprotein; GPI anchor |
| 952 | G00144 | (GlcN)1 (Ino-P)1; Glycoprotein; GPI anchor |
| 953 | G00145 | (GlcN)1 (Ino(acyl)-P)1; Glycoprotein; GPI anchor |
| 954 | G00146 | (GlcN)1 (Ino(acyl)-P)1 (Man)1; Glycoprotein; GPI anchor |
| 955 | G00147 | (GlcN)1 (Ino(acyl)-P)1 (Man)1 (EtN)1 (P)1; Glycoprotein; GPI anchor |
| 956 | G00148 | (GlcN)1 (Ino(acyl)-P)1 (Man)2 (EtN)1 (P)1; Glycoprotein; GPI anchor |
| 957 | G00149 | (GlcN)1 (Ino(acyl)-P)1 (Man)3 (EtN)1 (P)1; Glycoprotein; GPI anchor |
| 958 | G00151 | (GlcN)1 (Ino(acyl)-P)1 (Man)4 (EtN)3 (P)3; Glycoprotein; GPI anchor |
| 959 | G00154 | (Xyl)1 (Ser)1; Glycoprotein; Glycosaminoglycan |
| 960 | G00155 | (Gal)1 (Xyl)1 (Ser)1; Glycoprotein; Glycosaminoglycan |
| 961 | G00156 | (Gal)2 (Xyl)1 (Ser)1; Glycoprotein; Glycosaminoglycan |
| 962 | G00157 | (Gal)2 (GlcA)1 (Xyl)1 (Ser)1; Glycoprotein; Glycosaminoglycan |
| 963 | G00158 | (Gal)2 (GalNAc)1 (GlcA)1 (Xyl)1 (Ser)1; Glycoprotein; |
| | | Glycosaminoglycan |
| 964 | G00159 | (Gal)2 (GalNAc)1 (GlcA)2 (Xyl)1 (Ser)1; Glycoprotein; |
| | | Glycosaminoglycan |
| 965 | G00160 | (Gal)2 (GalNAc)2 (GlcA)2 (Xyl)1 (Ser)1; Glycoprotein; |
| | | Glycosaminoglycan |
| 966 | G00162 | (Gal)2 (GlcA)1 (GlcNAc)1 (Xyl)1 (Ser)1; Glycoprotein; |
| | | Glycosaminoglycan |
| 967 | G00163 | (Gal)2 (GlcA)2 (GlcNAc)1 (Xyl)1 (Ser)1; Glycoprotein; |
| | | Glycosaminoglycan |
| 968 | G00164 | (Gal)2 (GlcA)2 (GlcNAc)2 (Xyl)1 (Ser)1; Glycoprotein; |
| | | Glycosaminoglycan |
| 969 | G00166 | Fucosyl-GM1; (Gal)2 (GalNAc)1 (Glc)1 (LFuc)1 (Neu5Ac)1 (Cer)1; |
| | | Glycolipid; Sphingolipid |
| 970 | G00171 | (Glc)2 (GlcNAc)2 (Man)9 (Asn)1; Glycoprotein; N-Glycan |
| 971 | G04561 | Monofucosyllactoisooctaosylceramide; (Gal)4 (Glc)1 (GlcNAc)3 (LFuc)1 |
| | | (Cer)1; Glycolipid; Sphingolipid |
| 972 | G10511 | Monofucosyllactoisooctaosylceramide; (Gal)4 (Glc)1 (GlcNAc)3 (LFuc)1 |
| | | (Cer)1; Glycolipid; Sphingolipid |
| 973 | G10526 | (GlcNAc)2 (Man)4 (PP-Dol)1; Glycoprotein; N-Glycan |
| 974 | G10595 | (GlcNAc)2 (Man)6 (PP-Dol)1; Glycoprotein; N-Glycan |
| 975 | G10596 | (GlcNAc)2 (Man)7 (PP-Dol)1; Glycoprotein; N-Glycan |
| 976 | G10597 | (GlcNAc)2 (Man)8 (PP-Dol)1; Glycoprotein; N-Glycan |
| 977 | G10598 | (Glc)1 (GlcNAc)2 (Man)9 (PP-Dol)1; Glycoprotein; N-Glycan |
| 978 | G10599 | (Glc)2 (GlcNAc)2 (Man)9 (PP-Dol)1; Glycoprotein; N-Glycan |
| 979 | G10610 | UDP-N-acetyl-D-glucosamine; UDP-N-acetylglucosamine; (UDP- |
| | | GlcNAc)1 |
| 980 | G10611 | UDP-N-acetyl-D-galactosamine; UDP-N-acetylgalactosamine; (UDP- |
| | | GalNAc)1 |
| 981 | G10617 | Dolichyl phosphate D-mannose; Dolichyl D-mannosyl phosphate; (Man)1 |
| | | (P-Dol)1 |
| 982 | G12396 | 6-(alpha-D-glucosaminyl)-1D-myo-inositol; (GlcN)1 (Ino)1 |

The foregoing description is intended to illustrate various aspects of the instant technology. It is not intended that the examples presented herein limit the scope of the appended claims. The invention now being fully described, it will be apparent to one of ordinary skill in the art that many changes and modifications can be made thereto without departing from the spirit or scope of the appended claims.

1. A method for identifying one or more metabolites associated with a disease, the method comprising:
obtaining a set of gene-expression data from diseased cells of an individual with the disease;
obtaining a reference set of gene-expression data from control cells;
assigning an expression status to each gene in the gene expression data that encodes a gene product, wherein the expression status for each gene is one of:
up-regulated in the diseased cells relative to the control cells;
down-regulated in the diseased cells relative to the control cells;
expressed by both the diseased cells and the control cells at statistically indistinguishable levels; and
not expressed by both the diseased cells and the control cells;
determining the effects of gene products on metabolite levels
for each metabolite in a list of human metabolites:
identify a set of gene products that have an effect on the metabolite;
using the expression status for the gene that encodes each gene product that has an effect on the metabolite, predict whether an intracellular level of the metabolite in the diseased cells is increased or decreased relative to its level in control cells;
identifying one or more of:
those metabolites whose intracellular level is predicted to be lower in diseased cells than in control cells; and
those metabolites whose intracellular level is predicted to be higher in diseased cells than in control cells,
as associated with the disease.

2. The method of claim 1, wherein the diseased cells are cancer cells.

3. The method of claim 1, wherein each gene that encodes a gene product has been identified from a database of gene function.

4. The method of claim 3, wherein each gene that encodes a gene product has been identified from a database of gene function in conjunction with a prediction of the function of the gene product.

5. The method of claim 1, wherein the disease is leukemia, and the one or more metabolites include: seleno-L-methionine, dehydroepiandrosterone, Menaquinone, α-hydroxystearic acid, 5,6-dimethylbenzimidazole, and 3-sulfino-L-alanine.

6. The method of claim 1, wherein the disease is ovarian cancer, and the one or more metabolites include: α-hydroxystearic acid, 5,6-dimethylbenzimidazole, and androsterone.

7. The method of claim 1, wherein the metabolite is associated with the disease by one or more of: binding to a regulatory region of an mRNA; activating a transcription factor by binding of the metabolite; regulating gene expression by accomplishing a post-translational modification; being produced by an enzyme; being consumed by an enzyme; and being transported by a small molecule transporter.

8. The method of claim 3, wherein the database of gene function contains information on metabolic pathways selected from the group consisting of: carbohydrate metabolism; energy metabolism; lipid metabolism; nucleotide metabolism; amino acid metabolism; metabolism of other amino acids; glycan biosynthesis and metabolism; biosynthesis of polyketides and nonribosomal peptides; metabolism of cofactors and vitamins; biosynthesis of secondary metabolites; and biodegradation and metabolism of xenobiotics.

9. The method of claim 1, wherein the prediction that an intracellular level of the metabolite in the diseased cells is decreased relative to its level in control cells is based on the following:
there is at least one gene encoding for a gene product able to decrease the intracellular level of the metabolite that is either similarly-regulated or up-regulated in the diseased cells relative to the control cells and
there is no gene encoding for a gene product able to increase the intracellular level of the metabolite that is either up-regulated or similarly-regulated in the diseased cells relative to the control cells or
there is no gene encoding for a gene product able to decrease the intracellular level of the metabolite that is down-regulated in diseased cells; and
either or both of the following applies:
there is at least one gene encoding for a gene product able to increase the intracellular level of the metabolite that is down-regulated in diseased cells; and
there is at least one gene encoding for a gene product able to decrease the intracellular level of the metabolite that is up-regulated in diseased cells.

10. The method of claim 1, wherein the prediction that an intracellular level of the metabolite in the diseased cells is increased relative to the level in control cells is based on the following:
there is at least one gene encoding for a gene product able to increase the intracellular level of the metabolite that is either similarly-regulated or up-regulated in the diseased cells relative to the control cells and
there is no gene encoding for a gene product able to increase the intracellular level of the metabolite that is down-regulated in the diseased cells relative to the control cells and
there is no gene encoding for a gene product able to decrease the intracellular level of the metabolite that is either similarly regulated or up-regulated in diseased cells; and
either or both of the following applies:
there is at least one gene encoding for a gene product able to increase the intracellular level of the metabolite that is up-regulated in diseased cells; and
there is at least one gene encoding for a gene product able to decrease the intracellular level of the metabolite that is down-regulated in diseased cells.

11. The method of claim 1, wherein the gene expression data are obtained in micro-array format.

12. The method of claim 1, wherein a gene product includes an enzyme or a small-molecule transporter.

13. The method of claim 1, wherein a gene product is an enzyme that either employs a metabolite as a substrate, or generates it as a product.

14. The method of claim 1, wherein a gene product is a small-molecule transporter that is responsible for transporting a metabolite in a metabolic pathway.

15. A method of determining a metabolite-based disease therapy, the method comprising:
identifying one or more metabolites associated with the disease, by the method of claim 1; and
administering said one or more metabolites to an individual with the disease.

16. A method of treating an individual with a disease, the method comprising:
administering to the individual a metabolite identified as associated with the disease by the method of claim 1, in an amount sufficient to produce a therapeutic effect.

17. A method of determining a metabolite-based disease therapy, the method comprising:
identifying one or more metabolites associated with the disease, by the method of claim 1; and
administering one or more drugs to change the levels of said one or more metabolites to an individual with the disease.

18. A method for identifying one or more metabolites associated with a disease, the method comprising:
comparing gene expression data from diseased cells to gene expression data from control cells in order to deduce genes that are differentially-regulated in the diseased cells relative to the control cells;
based on enzyme function and pathway data for all human metabolites that utilize the genes that are differentially-regulated in the disease cells, identifying one or more metabolites whose intracellular levels are lower in diseased cells than in control cells, and thereby associating the one or more metabolites with the disease.

19. A method for identifying one or more metabolites associated with a disease, the method comprising:
comparing gene expression data from diseased cells to gene expression data from control cells in order to deduce genes that are differentially-regulated in the diseased cells relative to the control cells;
based on enzyme function and pathway data for all human metabolites that utilize the genes that are differentially-regulated in the disease cells, identifying one or more metabolites whose intracellular levels are higher in diseased cells than in control cells, and thereby associating the one or more metabolites with the disease.

20. A computer readable medium, encoded with instructions for carrying out the method of claim 1.

21. A computer system, comprising:
an input/output device;
a processor; and a
memory,
wherein the memory is configured with instructions, executable by the processor, to carry out the method of claim 1, and to provide the results of the method to a user, via the input/output device.
